(19) United States
(12) Patent Application Publication
(10) Pub. No.: US 2025/0137075 A1
(43) Pub. Date: May 1, 2025

(54) VARIN GENES

(71) Applicant: Phylos Bioscience, Inc., Portland, OR (US)

(72) Inventors: Erica Bakker, Portland, OR (US); Alisha Holloway, Portland, OR (US); Annie Poczynek, Portland, OR (US)

(73) Assignee: Phylos Bioscience, Inc., Portland, OR (US)

(21) Appl. No.: 18/681,833

(22) PCT Filed: Aug. 4, 2022
(86) PCT No.: PCT/US2022/074558

§ 371 Date: Feb. 6, 2024

Related U.S. Application Data
(60) Provisional application No. 63/230,235, filed on Aug. 6, 2021.

(51) Int. Cl. C12Q 1/6895

(52) U.S. Cl. CPC C12Q 1/6895; C12Q 2600/13

(57) ABSTRACT

Provided herein is the identification of the causal gene KR associated with THCV, THCVA, CBDV, CBDVA, CBGV, CBGVA, CBCV, or CBCVA production in Cannabis plants. Also included are isolated cells, nucleic acids, transgenic Cannabis plants; methods of editing, and methods of using marker assisted selection to select and establish plant lines having modified THCV, THCVA, CBDV, CBDVA, CBGV, CBGVA, CBCV, or CBCVA activity.

### CROSS REFERENCE TO RELATED APPLICATIONS

This application claims priority benefit to U.S. provisional application No. 63/230,235, filed Aug. 6, 2021, the entire contents of which are hereby incorporated by reference.

### SEQUENCE LISTING REFERENCE

Pursuant to 37 CFR §§ 1.821-1.825, a Sequence Listing in the form of an ASCII-compliant text file (entitled “2004-2-WO1_ST26_Sequence_Listing.xml” created on Aug. 3, 2022 and 59 kilobytes in size), which will serve as both the paper copy required by 37 CFR § 1.821 (c) and the computer readable form (CRF) required by 37 CFR § 1.821 (e), is submitted concurrently with the instant application. The entire contents of the Sequence Listing are incorporated herein by reference.

### BACKGROUND OF THE INVENTION

Cannabis plants contain over a hundred known cannabinoids, which bind to endogenous endocannabinoid receptors. Varinolic cannabinoids, as known as varins, are a type of cannabinoid compounds having three carbon atoms in their alkyl side chain instead of the five carbon atom alkyl side chains more commonly associated with cannabinoids. Two such varins are tetrahydrocannabivarin (THCV) and cannabidivarin (CBDV), which are homologues of tetrahydrocannabinol (THC) and cannabidiol (CBD), respectively. Each varin has a unique pharmacological profile and distinct molecular targets.

Tetrahydrocannabivarin (THCV) is a homologue of tetrahydrocannabinol (THC) with a unique pharmacological profile and distinct molecular targets. THCV is a cannabinoid receptor type 1 antagonist and cannabinoid receptor type 2 partial agonist. Δ8-THCV has also been shown to be a CB1 antagonist, an agonist of GPR55 and I-α-lysophosphatidylinositol (LPI), and activator of 5HT1A receptors. THCV promises potential benefits across a broad set of applications.

Tetrahydrocannabivarinic acid (THCVA) is the carboxylated precursor to THCV, and the compound present in Cannabis varieties. As mentioned herein, phytocannabinoids such as THCV are synthesized in the plant as acid forms (e.g., THCVA), and while some decarboxylation does occur in the plant, it increases significantly post-harvest and the kinetics increase at high temperatures.

THCV and CBDV have potential benefits across a broad set of applications. Cannabis strains or extracts with high THCV levels, for example, can be used as an agent for anticonvulsant activity, obesity-associated glucose intolerance, appetite suppression, anxiety management for PTSD, diabetic neuropathy, and major neuropathic and pain related pathologies. Another THCV application is for treatment of Parkinson's Disease progression and symptoms. CBDV has been shown to have anti-epileptic and anticonvulsant activity. Cannabichromevarin (CBCV) is a non-psychoactive cannabinoid that is a homolog of CBC, and may have anticonvulsant activity. Cannabigerivarin (CBGV) is a non-psychoactive cannabinoid that is a homolog of CBG, and may have analgesic and anti-inflammatory properties.

Research and development as well as the sale of varin products has been limited due to low commonly occurring levels of varins, such as THCV, in Cannabis flower. The ability to produce Cannabis with high varin levels will create a platform for a new Cannabinoid category with differentiated, high margin products in both medical and recreational markets.

The most common way to create Cannabis varieties having modified varin activity is the use of traditional methods of breeding that select for segregated traits over multiple generations. However, traditional breeding methods are laborious and time-consuming. The invention described herein utilizes discovered markers that closely segregate with the KR/FabG1/FabG gene for selecting varin attributes.

The KR gene pt/mtKR/FabG1 (β ketoacyl-acyl carrier protein (ACP) reductase, At1g24360) is also referred to as 3-oxoacyl-[acyl-carrier-protein] reductase. KR functions together with enoyl-ACP reductase (pt/mtER) to catalyze two of the reactions that constitute the core four-reaction cycle of the fatty acid biosynthesis (FAS) system, which iteratively elongates the acyl-chain by two carbon atoms per cycle (Guan et al. 2020, Plant Physiology 183 (2): 517-529). In Cannabis, plastid fatty acid biosynthesis forms the precursor for the acyl chain in THC and THCV (Welling et al. 2019, Scientific Reports 9(1): 1-13). Allelic variation of KR likely produces a KR variant that results in a shorter propyl (3-carbon) side chain found in THCV instead of a pentyl (5-carbon) group found in THC.

The invention described herein solves the laborious and time-consuming issues of traditional breeding methods by providing Cannabis breeders with a specific and efficient method for creating Cannabis plants having modified varin activity, including increased activity of one or more of THCV, CBDV, CBCV, or CBGV.

### SUMMARY OF THE INVENTION

The present teachings relate to the identification of markers and causal genes responsible for modified varin production in Cannabis. In an embodiment, a transgenic Cannabis plant whose genome comprises one or more amino acid substitutions of at least a portion of an endogenous KR gene and wherein the Cannabis plant comprises modified varin levels is provided. In an embodiment, the endogenous KR gene comprises a genomic nucleic acid sequence having at least 90% sequence identity to SEQ ID NO: 14 or a protein coding amino acid sequence having at least 90% sequence identity to SEQ ID NO:17. In an embodiment, the endogenous KR gene comprises one or more of: a nucleotide polymorphism at position 46 of SEQ ID NO: 14 that results in an Arginine to Glycine amino acid substitution at position 16 of SEQ ID NO:17; a nucleotide polymorphism at position 107 of SEQ ID NO:14 that results in an Serine to Phenylalanine amino acid substitution at position 36 of SEQ ID NO:17; a nucleotide polymorphism at position 109 of SEQ ID NO: 14 that results in a Glycine to Serine amino acid substitution at position 37 of SEQ ID NO: 17; a nucleotide polymorphism at position 188 of SEQ ID NO: 14 that results in a Glycine to Valine amino acid substitution at position 63 of SEQ ID NO:17; a nucleotide polymorphism at position 191 of SEQ ID NO:14 that results in an Alanine to Glutamic Acid amino acid substitution at position 64 of SEQ ID NO: 17; a nucleotide polymorphism at position 194 of SEQ ID NO: 14 that results in an Serine to Threonine amino acid substitution at position 65 of SEQ ID NO:17; a nucleotide polymorphism at position 229 of SEQ ID NO:14 that results in a Valine to Leucine amino acid substitution at position 77 of SEQ ID NO: 17; a nucleotide polymorphism at position 274 of SEQ ID NO: 14 that results in a Glycine to Arginine amino acid substitution at position 92 of SEQ ID NO:17; a nucleotide polymorphism at position 335 of SEQ ID NO: 14 that results in an Arginine to Lysine amino acid substitution at position 112 of SEQ ID NO: 17; a nucleotide polymorphism at position 412 of SEQ ID NO: 14 that results in a Valine to Isoleucine amino acid substitution at position 138 of SEQ ID NO:17; a nucleotide polymorphism at position 443 of SEQ ID NO: 14 that results in an Isoleucine to Threonine amino acid substitution at position 148 of SEQ ID NO: 17; a nucleotide polymorphism at position 449 of SEQ ID NO: 14 that results in a Threonine to Isoleucine amino acid substitution at position 150 of SEQ ID NO:17; a nucleotide polymorphism at position 519 of SEQ ID NO: 14 that results in an Isoleucine to Methionine amino acid substitution at position 173 of SEQ ID NO:17; a nucleotide polymorphism at position 686 of SEQ ID NO: 14 that results in a Tyrosine to Cysteine amino acid substitution at position 229 of SEQ ID NO:17; a nucleotide polymorphism at position 727 of SEQ ID NO: 14 that results in an Isoleucine to Valine amino acid substitution at position 243 of SEQ ID NO:17; or a nucleotide polymorphism at position 908 of SEQ ID NO:14 that results in a Glycine to Aspartic Acid amino acid substitution at position 303 of SEQ ID NO:17.

In an embodiment, the KR gene of the transgenic Cannabis plant is heterozygous and comprises an A and G nucleotide at position 46 of SEQ ID NO: 14; a C and T nucleotide at position 107 of SEQ ID NO: 14; a G and A nucleotide at position 109 of SEQ ID NO: 14; a G and T nucleotide at position 188 of SEQ ID NO:14; a C and A nucleotide at position 191 of SEQ ID NO: 14; a G and C nucleotide at position 194 of SEQ ID NO: 14; a G and T nucleotide at position 229 of SEQ ID NO: 14; a G and A nucleotide at position 274 of SEQ ID NO:14; a G and A nucleotide at position 335 of SEQ ID NO: 14; a G and A nucleotide at position 412 of SEQ ID NO: 14; a T and C nucleotide at position 443 of SEQ ID NO:14; a C and T nucleotide at position 449 of SEQ ID NO:14; a T and G nucleotide at position 519 of SEQ ID NO: 14; an A and G nucleotide at position 686 of SEQ ID NO:14; an A and G nucleotide at position 727 of SEQ ID NO: 14; or a G and A nucleotide at position 908 of SEQ ID NO: 14.

In an embodiment, the modified varin of the transgenic Cannabis plant is elevated total varin. In an embodiment, the elevated total varin is at least 2.0%. In an embodiment, the elevated total varin is between 4.2% and 14.3%. In an embodiment, the modified varin is an increased varin ratio. In an embodiment, the increased varin ratio is at least 0.39. In an embodiment, the increased varin ratio is between 0.39 and 6.7. In an embodiment, the varin is one or more of tetrahydrocannabivarin (THCV), cannabigerivarin (CBGV), cannabichromevarin (CBCV), or cannabidivarin (CBDV).

In an embodiment, a cell isolated from the transgenic Cannabis plant is provided. In an embodiment, a Cannabis product made from the transgenic plant is provided.

In another embodiment, an isolated nucleic acid sequence encoding one or more amino acids substitution of at least a portion of an endogenous Cannabis KR gene is provided. In an embodiment, the endogenous Cannabis KR gene comprises a genomic nucleic acid sequence having at least 90% sequence identity to SEQ ID NO: 14 or a protein coding amino acid sequence having at least 90% sequence identity to SEQ ID NO:17. In an embodiment, the endogenous Cannabis KR gene comprises one or more of: a nucleotide polymorphism at position 46 of SEQ ID NO: 14 that results in an Arginine to Glycine amino acid substitution at position 16 of SEQ ID NO:17; a nucleotide polymorphism at position 107 of SEQ ID NO:14 that results in an Serine to Phenylalanine amino acid substitution at position 36 of SEQ ID NO:17; a nucleotide polymorphism at position 109 of SEQ ID NO: 14 that results in a Glycine to Serine amino acid substitution at position 37 of SEQ ID NO: 17; a nucleotide polymorphism at position 188 of SEQ ID NO:14 that results in a Glycine to Valine amino acid substitution at position 63 of SEQ ID NO:17; a nucleotide polymorphism at position 191 of SEQ ID NO:14 that results in an Alanine to Glutamic Acid amino acid substitution at position 64 of SEQ ID NO:17; a nucleotide polymorphism at position 194 of SEQ ID NO:14 that results in an Serine to Threonine amino acid substitution at position 65 of SEQ ID NO: 17; a nucleotide polymorphism at position 229 of SEQ ID NO:14 that results in a Valine to Leucine amino acid substitution at position 77 of SEQ ID NO: 17; a nucleotide polymorphism at position 274 of SEQ ID NO: 14 that results in a Glycine to Arginine amino acid substitution at position 92 of SEQ ID NO:17; a nucleotide polymorphism at position 335 of SEQ ID NO: 14 that results in an Arginine to Lysine amino acid substitution at position 112 of SEQ ID NO:17; a nucleotide polymorphism at position 412 of SEQ ID NO: 14 that results in a Valine to Isoleucine amino acid substitution at position 138 of SEQ ID NO:17; a nucleotide polymorphism at position 443 of SEQ ID NO: 14 that results in an Isoleucine to Threonine amino acid substitution at position 148 of SEQ ID NO: 17; a nucleotide polymorphism at position 449 of SEQ ID NO: 14 that results in a Threonine to Isoleucine amino acid substitution at position 150 of SEQ ID NO:17; a nucleotide polymorphism at position 519 of SEQ ID NO: 14 that results in an Isoleucine to Methionine amino acid substitution at position 173 of SEQ ID NO:17; a nucleotide polymorphism at position 686 of SEQ ID NO:14 that results in a Tyrosine to Cysteine amino acid substitution at position 229 of SEQ ID NO:17; a nucleotide polymorphism at position 727 of SEQ ID NO:14 that results in an Isoleucine to Valine amino acid substitution at position 243 of SEQ ID NO:17; or a nucleotide polymorphism at position 908 of SEQ ID NO:14 that results in a Glycine to Aspartic Acid amino acid substitution at position 303 of SEQ ID NO:17. In an embodiment, the endogenous Cannabis KR gene is heterozygous and comprises an A and G nucleotide at position 46 of SEQ ID NO: 14; a C and T nucleotide at position 107 of SEQ ID NO: 14; a G and A nucleotide at position 109 of SEQ ID NO:14; a G and T nucleotide at position 188 of SEQ ID NO:14; a C and A nucleotide at position 191 of SEQ ID NO: 14; a G and C nucleotide at position 194 of SEQ ID NO: 14; a G and T nucleotide at position 229 of SEQ ID NO:14; a G and A nucleotide at position 274 of SEQ ID NO:14; a G and A nucleotide at position 335 of SEQ ID NO: 14; a G and A nucleotide at position 412 of SEQ ID NO: 14; a T and C nucleotide at position 443 of SEQ ID NO: 14; a C and T nucleotide at position 449 of SEQ ID NO:14; a T and G nucleotide at position 519 of SEQ ID NO: 14; an A and G nucleotide at position 686 of SEQ ID NO: 14; an A and G nucleotide at position 727 of SEQ ID NO: 14; or a G and A nucleotide at position 908 of SEQ ID NO:14. In an embodiment, an isolated cell whose genome comprises the nucleic acid sequence is provided.

In another embodiment, a method of making a Cannabis plant with modified varin levels is provided. The method comprises replacing a nucleotide present within an endogenous KR gene with the isolated nucleic acid. In an embodiment, the endogenous KR gene comprises one or more of a nucleotide polymorphism at position 46 of SEQ ID NO: 14 that results in an Arginine to Glycine amino acid substitution at position 16 of SEQ ID NO: 17; a nucleotide polymorphism at position 107 of SEQ ID NO:14 that results in an Serine to Phenylalanine amino acid substitution at position 36 of SEQ ID NO:17; a nucleotide polymorphism at position 109 of SEQ ID NO:14 that results in a Glycine to Serine amino acid substitution at position 37 of SEQ ID NO: 17; a nucleotide polymorphism at position 188 of SEQ ID NO: 14 that results in a Glycine to Valine amino acid substitution at position 63 of SEQ ID NO:17; a nucleotide polymorphism at position 191 of SEQ ID NO: 14 that results in an Alanine to Glutamic Acid amino acid substitution at position 64 of SEQ ID NO: 17; a nucleotide polymorphism at position 194 of SEQ ID NO: 14 that results in an Serine to Threonine amino acid substitution at position 65 of SEQ ID NO: 17; a nucleotide polymorphism at position 229 of SEQ ID NO:14 that results in a Valine to Leucine amino acid substitution at position 77 of SEQ ID NO:17; a nucleotide polymorphism at position 274 of SEQ ID NO. 14 that results in a Glycine to Arginine amino acid substitution at position 92 of SEQ ID NO:17; a nucleotide polymorphism at position 335 of SEQ ID NO:14 that results in an Arginine to Lysine amino acid substitution at position 112 of SEQ ID NO:17; a nucleotide polymorphism at position 412 of SEQ ID NO: 14 that results in a Valine to Isoleucine amino acid substitution at position 138 of SEQ ID NO:17; a nucleotide polymorphism at position 443 of SEQ ID NO: 14 that results in an Isoleucine to Threonine amino acid substitution at position 148 of SEQ ID NO: 17; a nucleotide polymorphism at position 449 of SEQ ID NO: 14 that results in a Threonine to Isoleucine amino acid substitution at position 150 of SEQ ID NO: 17; a nucleotide polymorphism at position 519 of SEQ ID NO: 14 that results in an Isoleucine to Methionine amino acid substitution at position 173 of SEQ ID NO:17; a nucleotide polymorphism at position 686 of SEQ ID NO: 14 that results in a Tyrosine to Cysteine amino acid substitution at position 229 of SEQ ID NO: 17; a nucleotide polymorphism at position 727 of SEQ ID NO: 14 that results in an Isoleucine to Valine amino acid substitution at position 243 of SEQ ID NO: 17; or a nucleotide polymorphism at position 908 of SEQ ID NO:14 that results in a Glycine to Aspartic Acid amino acid substitution at position 303 of SEQ ID NO:17. In an embodiment, he KR gene is heterozygous and comprises an A and G nucleotide substitution at position 46 of SEQ ID NO: 14; a C and T nucleotide substitution at position 107 of SEQ ID NO: 14; a G and A nucleotide substitution at position 109 of SEQ ID NO:14; a G and T nucleotide substitution at position 188 of SEQ ID NO: 14; a C and A nucleotide substitution at position 191 of SEQ ID NO: 14; a G and C nucleotide substitution at position 194 of SEQ ID NO: 14; a G and T nucleotide substitution at position 229 of SEQ ID NO:14; a G and A nucleotide substitution at position 274 of SEQ ID NO: 14; a G and A nucleotide substitution at position 335 of SEQ ID NO: 14; a G and A nucleotide substitution at position 412 of SEQ ID NO: 14; a T and C nucleotide substitution at position 443 of SEQ ID NO: 14; a C and T nucleotide substitution at position 449 of SEQ ID NO: 14; a T and G nucleotide substitution at position 519 of SEQ ID NO: 14; an A and G nucleotide substitution at position 686 of SEQ ID NO: 14; an A and G nucleotide substitution at position 727 of SEQ ID NO:14; or a G and A nucleotide substitution at position 908 of SEQ ID NO: 14. In an embodiment, the modified varin of the Cannabis plant is elevated total varin. In an embodiment, the elevated total varin is at least 2.0%. In an embodiment, the elevated total varin is between 4.2% and 14.3%. In an embodiment, the modified varin is an increased varin ratio. In an embodiment, the increased varin ratio is at least 0.39. In an embodiment, the increased varin ratio is between 0.39 and 6.7. In an embodiment, the varin is one or more of tetrahydrocannabivarin (THCV), cannabigerivarin (CBGV), cannabichromevarin (CBCV), or cannabidivarin (CBDV). In an embodiment, the replacing comprises gene editing. In an embodiment, the gene editing comprises CRISPR technology.

In another embodiment, a method for selecting one or more plants having modified varin levels is provided. The method comprises i) obtaining nucleic acids from a sample plant(s) or their germplasm; (ii) detecting one or more markers that indicate a modified varin level phenotype, (iii) indicating the modified varin level phenotype, and (iv) selecting the one or more plants indicating the modified varin level phenotype. In an embodiment, the sample plant(s) is a progeny plant obtained from a cross between a first plant and a second plant wherein the first plant has modified varin levels and the second plant either (a) does not have modified varin levels, or (b) has modified varin leves with progeny that do not segregate modified varin levels.

In an embodiment, the one or more markers comprises a polymorphism at position 51 of any one or more of SEQ ID NO: 19; SEQ ID NO:20; SEQ ID NO:21; SEQ ID NO:22; SEQ ID NO:23; SEQ ID NO:24; SEQ ID NO:25; SEQ ID NO:26; SEQ ID NO:27; SEQ ID NO:28; SEQ ID NO:29; SEQ ID NO:30; SEQ ID NO:31; SEQ ID NO:32; SEQ ID NO:33; or SEQ ID NO:34. In an embodiment, the nucleotide position comprises an A/G or G/G genotype at position 51 of SEQ ID NO: 19; a C/T or T/T genotype at position 51 of SEQ ID NO:20; a G/A or A/A genotype at position 51 of SEQ ID NO:21; a G/T or T/T genotype at position 51 of SEQ ID NO:22; a C/A or A/A genotype at position 51 of SEQ ID NO:23; a G/C or C/C genotype at position 51 of SEQ ID NO: 24; a G/T or T/T genotype at position 51 of SEQ ID NO:25; a G/A or A/A genotype at position 51 of SEQ ID NO:26; a G/A or A/A genotype at position 51 of SEQ ID NO:27; a G/A or A/A genotype at position 51 of SEQ ID NO:28; a T/C or C/C genotype at position 51 of SEQ ID NO: 29; a C/T or T/T genotype at position 51 of SEQ ID NO:30; a T/G or G/G genotype at position 51 of SEQ ID NO:31; an A/G or G/G genotype at position 51 of SEQ ID NO:32; an A/G or G/G genotype at position 51 of SEQ ID NO:33; or a G/A or A/A genotype at position 51 of SEQ ID NO:34. In an embodiment, the selecting comprises marker assisted selection. In an embodiment, the detecting comprises an oligonucleotide probe. In an embodiment, the one or more plants comprises the indicated modified varin level phenotype to produce one or more F1 or additional progeny plants, wherein at least one of the F1 or additional progeny plants comprises the indicated modified varin level phenotype. In an embodiment, the crossing comprises selfing, sibling crossing, or backcrossing. In an embodiment, the at least one additional progeny plant comprising the indicated modified varin level phenotype comprises an F2-F7 progeny plant. In an embodiment, the selfing, sibling crossing, or backcrossing comprises marker-assisted selection for at least two generations. In an embodiment, the modified varin is elevated total varin. In an embodiment, the elevated total varin is at least 2.0%. In an embodiment, the elevated total varin is between 4.2% and 14.3%. In an embodiment, the modified varin is an increased varin ratio. In an embodiment, the increased varin ratio is at least 0.39. In an embodiment, the increased varin ratio is between 0.39 and 6.7. In an embodiment, the varin is one or more of tetrahydrocannabivarin (THCV), cannabigerivarin (CBGV), cannabichromevarin (CBCV), or cannabidivarin (CBDV). In an embodiment, the plant comprises a Cannabis plant.

### BRIEF DESCRIPTION OF THE DRAWING

The skilled artisan will understand that the drawings, described below, are for illustration purposes only. The drawings are not intended to limit the scope of the present teachings in any way.

FIG. 1 illustrates a schematic representation of the location of amino acid substitution inside (black triangles) and outside (white triangles) conserved domains (dark gray box=transit peptide; light gray boxes=nucleotide [NADP] binding domains) of the KR protein sequence. Amino acid substitutions observed for varin producing accession 21VLP5-1-101 are represented by black and white triangles pointing upwards, downwards pointing black triangles represent amino acid substitutions observed for varin producing accessions 21TX1-60 (both downward pointing black triangles) and 20VLP2-6-15 (only the second downward pointing black triangle since this accession had missing data for the transit peptide domain).

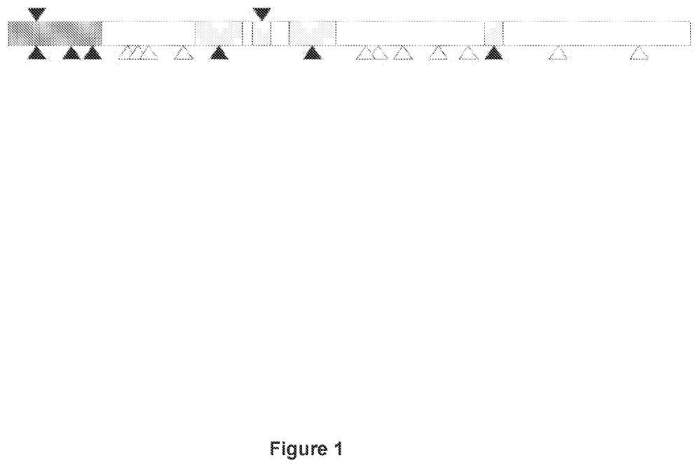

### DETAILED DESCRIPTION OF THE INVENTION

These and other features of the present teachings will become more apparent from the description herein. While the present teachings are described in conjunction with various embodiments, it is not intended that the present teachings be limited to such embodiments. On the contrary, the present teachings encompass various alternatives, modifications, and equivalents, as will be appreciated by those of skill in the art.

The present teachings relate generally to methods of producing Cannabis varieties having modified varin activity, including high THCV, CBDV, CBCV, or CBGV concentrations.

The terminology used in the disclosure herein is for the purpose of describing particular embodiments only and is not intended to limit the disclosure. As used in the description of the embodiments of the disclosure and the appended claims, the singular forms “a”, “an” and “the” are intended to include the plural forms as well, unless the context clearly indicates otherwise. Also, as used herein, “and/or” refers to and encompasses any and all possible combinations of one or more of the associated listed items. Furthermore, the term “about,” as used herein when referring to a measurable value such as an amount of a compound, amount, dose, time, temperature, for example, is meant to encompass variations of 20%, 10%, 5%, 1%, 0.5%, or even 0.1% of the specified amount. It will be further understood that the terms “comprises” and/or “comprising.” when used in this specification, specify the presence of stated features, integers, steps, operations, elements, and/or components, but do not preclude the presence or addition of one or more other features, integers, steps, operations, elements, components, and/or groups thereof. Unless otherwise defined, all terms, including technical and scientific terms used in the description, have the same meaning as commonly understood by one of ordinary skill in the art to which this disclosure belongs.

### Definitions

The term “Abacus” or the phrase “Abacus Cannabis reference genome” as used herein refers to the Cannabis reference genome known as the Abacus reference genome (version CsaAba2).

The term “acidic cannabinoid” refers to a cannabinoid having one or more carboxylic acid functional groups. Examples of acidic cannabinoids include, but are not limited to, tetrahydrocannabinolic acid (THCA), cannabidiolic acid (CBDA), tetrahydrocannabivarinic acid (TCHVA), and cannabichromenic acid (CBC). Acidic cannabinoids are frequently the predominant cannabinoids found in raw (i.e., unprocessed) Cannabis plant material.

The term “alternative nucleotide call” is a nucleotide polymorphism relative to a reference nucleotide for a SNP marker that is significantly associated with the causative SNP(s) that confer(s) a desired phenotype.

The term “backcrossing” or “to backcross” refers to the crossing of an F1 hybrid with one of the original parents. A backcross is used to maintain the identity of one parent (species) and to incorporate a particular trait from a second parent (species). The best strategy is to cross the F1 hybrid back to the parent possessing the most desirable traits. Two or more generations of backcrossing may be necessary, but this is practical only if the desired characteristic or trait is present in the F1.

The term “beneficial” as used herein refers to an allele conferring a modified varin activity phenotype.

The term “CBCV” means cannabichromevarin.

The term “CBCVA” means cannabichromevarinic acid.

The term “CBDV” means cannabidivarin.

The term “CBDVA” means cannabidivarinic acid.

The term “CBGV” means cannabigerivarin.

The term “CBGVA” means cannabigerivarinic acid.

The term “Cannabis” refers to plants of the genus Cannabis, including Cannabis sativa, and subspecies, Cannabis sativa indica, and Cannabis sativa ruderalis. Hemp is a type of Cannabis having low levels of tetrahydrocannabinol.

The term “cell” refers to a prokaryotic or eukaryotic cell, including plant cells, capable of replicating DNA, transcribing RNA, translating polypeptides, and secreting proteins.

The term “coding sequence” refers to a DNA sequence which codes for a specific amino acid sequence. “Regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include, but are not limited to, promoters, translation leader sequences, introns, and polyadenylation recognition sequences.

The terms “construct,” “plasmid,” “vector,” and “cassette” refer to an extra chromosomal element often carrying genes that are not part of the central metabolism of the cell, and usually in the form of circular double-stranded DNA fragments. Such elements may be autonomously replicating sequences, genome integrating sequences, phage or nucleotide sequences, linear or circular, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell. The term “recombinant DNA construct” or “recombinant expression construct” is used interchangeably and refers to a discrete polynucleotide into which a nucleic acid sequence or fragment can be moved. Preferably, it is a plasmid vector or a fragment thereof comprising the promoters of the present invention. The choice of plasmid vector is dependent upon the method that will be used to transform host plants. The skilled artisan is well aware of the genetic elements that must be present on the plasmid vector in order to successfully transform, select and propagate host cells containing the chimeric gene. The skilled artisan will also recognize that different independent transformation events will result in different levels and patterns of expression (Jones et al., EMBO J. 4:2411-2418 (1985); De Almeida et al., Mol. Gen. Genetics 218:78-86 (1989)), and thus that multiple events must be screened in order to obtain lines displaying the desired expression level and pattern. Such screening may be accomplished by PCR and Southern analysis of DNA, RT-PCR and Northern analysis of mRNA expression, Western analysis of protein expression, or phenotypic analysis.

The term “cross”, “crossing”, “cross pollination” or “cross-breeding” refer to the process by which the pollen of one flower on one plant is applied (artificially or naturally) to the ovule (stigma) of a flower on another plant. Backcrossing is a process in which a breeder repeatedly crosses hybrid progeny, for example a first generation hybrid (F1), back to one of the parents of the hybrid progeny. Backcrossing can be used to introduce one or more single locus conversions from one genetic background into another.

The term “cultivar” means a group of similar plants that by structural features and performance (e.g., morphological and physiological characteristics) can be identified from other varieties within the same species. Furthermore, the term “cultivar” variously refers to a variety, strain or race of plant that has been produced by horticultural or agronomic techniques and is not normally found in wild populations. The terms cultivar, variety, strain, plant and race are often used interchangeably by plant breeders, agronomists and farmers.

The term “detect” or “detecting” refers to any of a variety of methods for determining the presence of a nucleic acid.

The term “donor plants” refers to the parents of a variety which contains the gene or trait of interest which is desired to be introduced into a second variety (e.g., “recipient plants”).

The term “expression” or “gene expression” relates to the process by which the coded information of a nucleic acid transcriptional unit (including, e.g., genomic DNA) is converted into an operational, non-operational, or structural part of a cell, often including the synthesis of a protein. Gene expression can be influenced by external signals; for example, exposure of a cell, tissue, or organism to an agent that increases or decreases gene expression. Expression of a gene can also be regulated anywhere in the pathway from DNA to RNA to protein. Regulation of gene expression occurs, for example, through controls acting on transcription, translation, RNA transport and processing, degradation of intermediary molecules such as mRNA, or through activation, inactivation, compartmentalization, or degradation of specific protein molecules after they have been made, or by combinations thereof. Gene expression can be measured at the RNA level or the protein level by any method known in the art, including, without limitation, Northern blot, RT-PCR, Western blot, or in vitro, in situ, or in vivo protein activity assay(s). Elevated levels refers to higher than average levels of gene expression in comparison to a reference genome, e.g., the Abacus reference genome.

The term “functional” as used herein refers to DNA or amino acid sequences which are of sufficient size and sequence to have the desired function (i.e. the ability to cause expression of a gene resulting in gene activity expected of the gene found in a reference genome, e.g., the Abacus reference genome.)

The term “gene” refers to a nucleic acid fragment that expresses a specific protein, including regulatory sequences preceding (5′ non-coding sequences) and following (3′ non-coding sequences) the coding sequence. “Native gene” refers to a gene as found in nature with its own regulatory sequences. “Chimeric gene” or “recombinant expression construct”, which are used interchangeably, refers to any gene that is not a native gene, comprising regulatory and coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. “Endogenous gene” refers to a native gene in its natural location in the genome of an organism. A “foreign” gene refers to a gene not normally found in the host organism, but that is introduced into the host organism by gene transfer. Foreign genes can comprise native genes inserted into a non-native organism, or chimeric genes. A “transgene” is a gene that has been introduced into the genome by a transformation procedure.

The term “genetic modification” or “genetic alteration” as used herein refers to a change from the wild-type or reference sequence of one or more nucleic acid molecules. Genetic modifications or alterations include without limitation, base pair substitutions, additions and deletions of at least one nucleotide from a nucleic acid molecule of known sequence. One type of gene modification may be gene silencing, which is a reduction or complete absence of gene expression.

The term “genome” as it applies to plant cells encompasses not only chromosomal DNA found within the nucleus, but organelle DNA found within subcellular components (e.g., mitochondrial, plastid) of the cell.

The term “genotype” refers to the genetic makeup of an individual cell, cell culture, tissue, organism (e.g., a plant), or group of organisms.

The term “germplasm” refers to genetic material of or from an individual (e.g., a plant), a group of individuals (e.g., a plant line, variety, or family), or a clone derived from a line, variety, species, or culture. The germplasm can be part of an organism or cell, or can be separate from the organism or cell. In general, germplasm provides genetic material with a specific molecular makeup that provides a physical foundation for some or all of the hereditary qualities of an organism or cell culture. As used herein, germplasm includes cells, seed or tissues from which new plants can be grown, as well as plant parts, such as leaves, stems, pollen, or cells that can be cultured into a whole plant.

The term “haplotype” refers to the genotype of a plant at a plurality of genetic loci, e.g., a combination of alleles or markers. Haplotype can refer to sequence polymorphisms at a particular locus, such as a single marker locus, or sequence polymorphisms at multiple loci along a chromosomal segment in a given genome. As used herein, a haplotype can be a nucleic acid region spanning two markers.

A plant is “homozygous” if the individual has only one type of allele at a given locus (e.g., a diploid individual has a copy of the same allele at a locus for each of two homologous chromosomes). An individual is “heterozygous” if more than one allele type is present at a given locus (e.g., a diploid individual with one copy each of two different alleles). The term “homogeneity” indicates that members of a group have the same genotype at one or more specific loci. In contrast, the term “heterogeneity” is used to indicate that individuals within the group differ in genotype at one or more specific loci.

The term “homozygous deletion” refers to the deletion of one or more complementary nucleotides.

The terms “hybridizing specifically to,” “specific hybridization,” and “selectively hybridize to” as used herein refer to the binding, duplexing, or hybridizing of a nucleic acid molecule preferentially to a particular nucleotide sequence under stringent conditions. The term “stringent conditions” refers to conditions under which a probe will hybridize preferentially to its target subsequence, and to a lesser extent to, or not at all to, other sequences. A “stringent hybridization” and “stringent hybridization wash conditions” in the context of nucleic acid hybridization (e.g., as in array, Southern or Northern hybridizations) are sequence dependent, and are different under different environmental parameters. An extensive guide to the hybridization of nucleic acids is found in, e.g., Tijssen (1993) Laboratory Techniques in Biochemistry and Molecular Biology—Hybridization with Nucleic Acid Probes part I, Ch. 2, “Overview of principles of hybridization and the strategy of nucleic acid probe assays,” Elsevier, N.Y. (“Tijssen”). Generally, highly stringent hybridization and wash conditions are selected to be about 5. degree. C. lower than the thermal melting point (T.sub.m) for the specific sequence at a defined ionic strength and pH. The T.sub.m is the temperature (under defined ionic strength and pH) at which 50% of the target sequence hybridizes to a perfectly matched probe. Very stringent conditions are selected to be equal to the T.sub.m for a particular probe. An example of stringent hybridization conditions for hybridization of complementary nucleic acids which have more than 100 complementary residues on an array or on a filter in a Southern or northern blot is 42. degree. C. using standard hybridization solutions (see, e.g., Sambrook and Russell (2001) Molecular Cloning: A Laboratory Manual (3rd ed.) Vol. 1-3, Cold Spring Harbor Laboratory, Cold Spring Harbor Press, NY, and detailed discussion, below).

The term “hybrid” refers to a variety or cultivar that is the result of a cross of plants of two different varieties. “F1 hybrid” refers to the first generation hybrid, “F2 hybrid” the second generation hybrid, “F3 hybrid” the third generation, and so on. A hybrid refers to any progeny that is either produced or developed.

As used herein, the term “inbreeding” refers to the production of offspring via the mating between relatives. The plants resulting from the inbreeding process are referred to herein as “inbred plants” or “inbreds.”

The terms “initiate transcription,” “initiate expression,” “drive transcription,” and “drive expression” are used interchangeably herein and all refer to the primary function of a promoter. As detailed throughout this disclosure, a promoter is a non-coding genomic DNA sequence, usually upstream (5′) to the relevant coding sequence, and its primary function is to act as a binding site for RNA polymerase and initiate transcription by the RNA polymerase. Additionally, there is “expression” of RNA, including functional RNA, or the expression of polypeptide for operably linked encoding nucleotide sequences, as the transcribed RNA ultimately is translated into the corresponding polypeptide.

The term “introduced” refers to a nucleic acid (e.g., expression construct) or protein into a cell. Introduced includes reference to the incorporation of a nucleic acid into a eukaryotic or prokaryotic cell where the nucleic acid may be incorporated into the genome of the cell, and includes reference to the transient provision of a nucleic acid or protein to the cell. Introduced includes reference to stable or transient transformation methods, as well as sexually crossing. Thus, “introduced” in the context of inserting a nucleic acid fragment (e.g., a recombinant DNA construct/expression construct) into a cell, means “transfection” or “transformation” or “transduction” and includes reference to the incorporation of a nucleic acid fragment into a eukaryotic or prokaryotic cell where the nucleic acid fragment may be incorporated into the genome of the cell (e.g., chromosome, plasmid, plastid or mitochondrial DNA), converted into an autonomous replicon, or transiently expressed (e.g., transfected mRNA).

The term “intron” refers to an intervening sequence in a gene that is transcribed into RNA but is then excised in the process of generating the mature mRNA. The term is also used for the excised RNA sequences. An “exon” is a portion of the sequence of a gene that is transcribed and is found in the mature messenger RNA derived from the gene, but is not necessarily a part of the sequence that encodes the final gene product.

The term “isolated” as used herein means having been removed from its natural environment, or removed from other compounds present when the compound is first formed. The term “isolated” embraces materials isolated from natural sources as well as materials (e.g., nucleic acids and proteins) recovered after preparation by recombinant expression in a host cell, or chemically-synthesized compounds such as nucleic acid molecules, proteins, and peptides.

The term “line” is used broadly to include, but is not limited to, a group of plants vegetatively propagated from a single parent plant, via tissue culture techniques or a group of inbred plants which are genetically very similar due to descent from a common parent(s). A plant is said to “belong” to a particular line if it (a) is a primary transformant (TO) plant regenerated from material of that line; (b) has a pedigree comprised of a TO plant of that line; or (c) is genetically very similar due to common ancestry (e.g., via inbreeding or selfing). In this context, the term “pedigree” denotes the lineage of a plant, e.g. in terms of the sexual crosses affected such that a gene or a combination of genes, in heterozygous (hemizygous) or homozygous condition, imparts a desired trait to the plant.

The term “KR/FabG1” or “KR/FabG1 gene” or “KR/FabG1 protein” or “KR” refers to Cannabis gene/gene product/protein known as β ketoacyl-acyl carrier protein (ACP) reductase, At1g24360, or 3-oxoacyl-[acyl-carrier-protein] reductase.

The term “marker,” “genetic marker,” “molecular marker,” “marker nucleic acid,” and “marker locus” refer to a nucleotide sequence or encoded product thereof (e.g., a protein) used as a point of reference when identifying a linked locus. A marker can be derived from genomic nucleotide sequence or from expressed nucleotide sequences (e.g., from a spliced RNA, a cDNA, etc.), or from an encoded polypeptide, and can be represented by one or more particular variant sequences, or by a consensus sequence. In another sense, a marker is an isolated variant or consensus of such a sequence. The term also refers to nucleic acid sequences complementary to or flanking the marker sequences, such as nucleic acids used as probes or primer pairs capable of amplifying the marker sequence. A “marker probe” is a nucleic acid sequence or molecule that can be used to identify the presence of a marker locus, e.g., a nucleic acid probe that is complementary to a marker locus sequence. Alternatively, in some aspects, a marker probe refers to a probe of any type that is able to distinguish (i.e., genotype) the particular allele that is present at a marker locus. A “marker locus” is a locus that can be used to track the presence of a second linked locus, e.g., a linked locus that encodes or contributes to expression of a phenotypic trait. For example, a marker locus can be used to monitor segregation of alleles at a locus, such as a QTL, that are genetically or physically linked to the marker locus. Thus, a “marker allele,” alternatively an “allele of a marker locus” is one of a plurality of polymorphic nucleotide sequences found at a marker locus in a population that is polymorphic for the marker locus. Other examples of such markers are restriction fragment length polymorphism (RFLP) markers, amplified fragment length polymorphism (AFLP) markers, single nucleotide polymorphisms (SNPs), microsatellite markers (e.g. SSRs), sequence-characterized amplified region (SCAR) markers, cleaved amplified polymorphic sequence (CAPS) markers or isozyme markers or combinations of the markers described herein which defines a specific genetic and chromosomal location.

The term “marker assisted selection” refers to the diagnostic process of identifying, optionally followed by selecting a plant from a group of plants using the presence of a molecular marker as the diagnostic characteristic or selection criterion. The process usually involves detecting the presence of a certain nucleic acid sequence or polymorphism in the genome of a plant.

The phrase “modified activity” or “modified expression” or “altered activity” or “altering expression” refers to the production of gene product(s) in organisms in amounts or proportions that differ from the amount of the gene product(s) produced by the corresponding wild-type organisms (i.e., expression is increased or decreased). The modified expression or activity can result in increases or decreases in amounts or levels of different compounds, including cannabinoids such as TCHV or CBDV.

The term “neutral cannabinoid” refers to a cannabinoid without carboxylic acid functional groups. Examples of neutral cannabinoids include, but are not limited to, THC, THCV, CBD, CBG, CBC, and CBN.

The term “offspring” refers to any plant resulting as progeny from a vegetative or sexual reproduction from one or more parent plants or descendants thereof. For instance an offspring plant may be obtained by cloning or selfing of a parent plant or by crossing two parent plants and includes selfings as well as the F1 or F2 or still further generations. An F1 is a first-generation offspring produced from parents at least one of which is used for the first time as donor of a trait, while offspring of second generation (F2) or subsequent generations (F3, F4, etc.) are specimens produced from selfings of F1's, F2's etc. An F1 may thus be (and usually is) a hybrid resulting from a cross between two true breeding parents (true-breeding is homozygous for a trait), while an F2 may be (and usually is) an offspring resulting from self-pollination of said F1 hybrids.

The term “oligonucleotide probe” refers to any kind of nucleotide molecule synthesized to match (i.e., be complementary to) a nucleotide sequence of interest which can be used to detect, analyse, and/or visualize said nucleotide sequence on a molecular level. An oligonucleotide probe according to the present disclosure generally refers to a molecule comprising several nucleotides, in general at least 10, 15, and even at least 20 nucleotides, for example, and having at least one label. Optionally, the oligonucleotide probe may also comprise any suitable non-nucleotide units and/or linking reagent which may be suitable to incorporate the label. It should be understood that the oligonucleotide probe has a length suitable to provide the required specificity. In general, the probe may be a DNA oligonucleotide probe or a RNA oligonucleotide probe. Further, it should also be understood that a nucleotide includes all kind of structures composed of a nucleobase (i.e. a nitrogenous base), a five carbon sugar which may be either a ribose, a 2′-deoxyribose, or any derivative thereof, and a phosphate group. The nucleobase and the sugar constitute a unit referred to as a nucleoside.

The terms “percent sequence identity” or “percent identity” or “identity” are used interchangeably to refer to a sequence comparison based on identical matches between correspondingly identical positions in the sequences being compared between two or more amino acid or nucleotide sequences. The percent identity refers to the extent to which two optimally aligned polynucleotide or peptide sequences are invariant throughout a window of alignment of components, e.g., nucleotides or amino acids. Hybridization experiments and mathematical algorithms known in the art may be used to determine percent identity. Many mathematical algorithms exist as sequence alignment computer programs known in the art that calculate percent identity. These programs may be categorized as either global sequence alignment programs or local sequence alignment programs.

The term “plant” refers to a whole plant and any descendant, cell, tissue, or part of a plant. A class of plant that can be used in the present invention is generally as broad as the class of higher and lower plants amenable to mutagenesis including angiosperms (monocotyledonous and dicotyledonous plants), gymnosperms, ferns and multicellular algae. Thus, “plant” includes dicot and monocot plants. The term “plant parts” include any part(s) of a plant, including, for example and without limitation: seed (including mature seed and immature seed); a plant cutting; a plant cell; a plant cell culture; a plant organ (e.g., pollen, embryos, flowers, fruits, shoots, leaves, roots, stems, and explants). A plant tissue or plant organ may be a seed, protoplast, callus, or any other group of plant cells that is organized into a structural or functional unit. A plant cell or tissue culture may be capable of regenerating a plant having the physiological and morphological characteristics of the plant from which the cell or tissue was obtained, and of regenerating a plant having substantially the same genotype as the plant. In contrast, some plant cells are not capable of being regenerated to produce plants. Regenerable cells in a plant cell or tissue culture may be embryos, protoplasts, meristematic cells, callus, pollen, leaves, anthers, roots, root tips, silk, flowers, kernels, ears, cobs, husks, or stalks. Plant parts include harvestable parts and parts useful for propagation of progeny plants. Plant parts useful for propagation include, for example and without limitation: seed; fruit; a cutting; a seedling; a tuber; and a rootstock. A harvestable part of a plant may be any useful part of a plant, including, for example and without limitation: flower; pollen; seedling; tuber; leaf; stem; fruit; seed; and root. A plant cell is the structural and physiological unit of the plant, comprising a protoplast and a cell wall. A plant cell may be in the form of an isolated single cell, or an aggregate of cells (e.g., a friable callus and a cultured cell), and may be part of a higher organized unit (e.g., a plant tissue, plant organ, and plant). Thus, a plant cell may be a protoplast, a gamete producing cell, or a cell or collection of cells that can regenerate into a whole plant. As such, a seed, which comprises multiple plant cells and is capable of regenerating into a whole plant, is considered a “plant cell” in embodiments herein. In an embodiment described herein are plants in the genus of Cannabis and plants derived thereof, which can be produced asexual or sexual reproduction.

The term “plant part” or “plant tissue” refers to any part of a plant including but not limited to, an embryo, shoot, root, stem, seed, stipule, leaf, petal, flower bud, flower, ovule, bract, trichome, branch, petiole, internode, bark, pubescence, tiller, rhizome, frond, blade, ovule, pollen, stamen. Plant part may also include certain extracts such as kief, oil, or hash which includes Cannabis trichomes or glands.

The terms “polynucleotide,” “polynucleotide sequence,” “nucleotide,” “nucleotide sequence,” “nucleic acid sequence,” “nucleic acid fragment,” and “isolated nucleic acid fragment” are used interchangeably herein. These terms encompass nucleotide sequences and the like. A polynucleotide may be a polymer of RNA or DNA that is single- or double-stranded, that optionally contains synthetic, non-natural or altered nucleotide bases. A polynucleotide in the form of a polymer of DNA comprises one or more segments of cDNA, genomic DNA, synthetic DNA, or mixtures thereof. Nucleotides (usually found in their 5′-monophosphate form) are referred to by a single letter designation as follows: “A” for adenylate or deoxyadenylate (for RNA or DNA, respectively), “C” for cytidylate or deoxycytidylate, “G” for guanylate or deoxyguanylate, “U” for uridylate, “T” for deoxythymidylate, “R” for purines (A or G), “Y” for pyrimidines (C or T), “K” for G or T, “H” for A or C or T, “I” for inosine, and “N” for any nucleotide. An “isolated polynucleotide” refers to a polymer of ribonucleotides (RNA) or deoxyribonucleotides (DNA) that is single- or double-stranded, optionally containing synthetic, non-natural or altered nucleotide bases. An isolated polynucleotide in the form of DNA may be comprised of one or more segments of cDNA, genomic DNA or synthetic DNA.

The term “polymorphism” or “nucleotide polymorphism” refers to a difference in the nucleotide or amino acid sequence of a given region as compared to a nucleotide or amino acid sequence in a homologous-region of another individual, in particular, a difference in the nucleotide of amino acid sequence of a given region which differs between individuals of the same species. A polymorphism is generally defined in relation to a reference sequence. Polymorphisms include single nucleotide differences, differences in sequence of more than one nucleotide, and single or multiple nucleotide insertions, inversions and deletions; as well as single amino acid differences, differences in sequence of more than one amino acid, and single or multiple amino acid insertions, inversions, and deletions.

The term “probe” or “nucleic acid probe,” as used herein, is defined to be a collection of one or more nucleic acid fragments whose specific hybridization to a nucleic acid sample comprising a region of interest can be detected. The probe may be unlabeled or labeled as described below so that its binding to the target nucleic acid of interest can be detected. What “probe” refers to specifically is clear from the context in which the word is used. The probe may also be isolated nucleic acids immobilized on a solid surface (e.g., nitrocellulose, glass, quartz, fused silica slides), as in an array. In some embodiments, the probe may be a member of an array of nucleic acids as described, for instance, in WO 96/17958. Techniques capable of producing high density arrays can also be used for this purpose (see, e.g., Fodor (1991) Science 767-773; Johnston (1998) Curr. Biol. 8: R171-R174; Schummer (1997) Biotechniques 23:1087-1092; Kern (1997) Biotechniques 23:120-124; U.S. Pat. No. 5,143,854). One of skill will recognize that the precise sequence of the particular probes described herein can be modified to a certain degree to produce probes that are “substantially identical” to the disclosed probes, but retain the ability to specifically bind to (i.e., hybridize specifically to) the same targets or samples as the probe from which they were derived (see discussion above). Such modifications are specifically covered by reference to the individual probes described herein.

The term “progeny” refers to any subsequent generation of a plant. Progeny is measured using the following nomenclature: F1 refers to the first generation progeny, F2 refers to the second generation progeny, F3 refers to the third generation progeny, and so on.

The term “promoter” refers to a nucleic acid fragment capable of controlling transcription of another nucleic acid fragment. A promoter is capable of controlling the expression of a coding sequence or functional RNA. Functional RNA includes, but is not limited to, transfer RNA (tRNA) and ribosomal RNA (rRNA). The promoter sequence consists of proximal and more distal upstream elements, the latter elements often referred to as enhancers. Accordingly, an “enhancer” is a DNA sequence that can stimulate promoter activity, and may be an innate element of the promoter or a heterologous element inserted to enhance the level or tissue-specificity of a promoter. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental conditions. New promoters of various types useful in plant cells are constantly being discovered; numerous examples may be found in the compilation by Okamuro and Goldberg (Biochemistry of Plants 15:1-82 (1989)). It is further recognized that because in most cases the exact boundaries of regulatory sequences have not been completely defined, DNA fragments of some variation may have identical promoter activity.

The terms “PCR” or “Polymerase Chain Reaction” refers to a technique for the synthesis of large quantities of specific DNA segments, consisting of a series of repetitive cycles (Perkin Elmer Cetus Instruments, Norwalk, Conn.). Typically, the double stranded DNA is heat denatured, the two primers complementary to the 3′ boundaries of the target segment are annealed at low temperature and then extended at an intermediate temperature. One set of these three consecutive steps comprises a cycle.

The term “protein” refers to amino acid polymers that contain at least five constituent amino acids that are covalently joined by peptide bonds. The constituent amino acids can be from the group of amino acids that are encoded by the genetic code, which include: alanine, valine, leucine, isoleucine, methionine, phenylalanine, tyrosine, tryptophan, serine, threonine, asparagine, glutamine, cysteine, glycine, proline, arginine, histidine, lysine, aspartic acid, and glutamic acid. As used herein, the term “protein” is synonymous with the related terms “peptide” and “polypeptide.”

The term “quantitative trait loci” or “QTL” refers to the genetic elements controlling a quantitative trait.

The term “reference plant” or “reference genome” refers to a wild-type or reference sequence that SNPs or other markers in a test sample can be compared to in order to detect a modification of the sequence in the test sample.

The terms “similar,” “substantially similar” and “corresponding substantially” as used herein refer to nucleic acid fragments wherein changes in one or more nucleotide bases do not affect the ability of the nucleic acid fragment to mediate gene expression or produce a certain phenotype. These terms also refer to modifications of the nucleic acid fragments of the instant invention such as deletion or insertion of one or more nucleotides that do not substantially alter the functional properties of the resulting nucleic acid fragment relative to the initial, unmodified fragment. It is therefore understood, as those skilled in the art will appreciate, that the invention encompasses more than the specific exemplary sequences. A “substantially homologous sequence” refers to variants of the disclosed sequences such as those that result from site-directed mutagenesis, as well as synthetically derived sequences. A substantially homologous sequence of the present invention also refers to those fragments of a particular promoter nucleotide sequence disclosed herein that operate to promote the constitutive expression of an operably linked heterologous nucleic acid fragment. These promoter fragments will comprise at least about 20 contiguous nucleotides, preferably at least about 50 contiguous nucleotides, more preferably at least about 75 contiguous nucleotides, even more preferably at least about 100 contiguous nucleotides of the particular promoter nucleotide sequence disclosed herein. The nucleotides of such fragments will usually comprise the TATA recognition sequence of the particular promoter sequence. Such fragments may be obtained by use of restriction enzymes to cleave the naturally occurring promoter nucleotide sequences disclosed herein; by synthesizing a nucleotide sequence from the naturally occurring promoter DNA sequence; or may be obtained through the use of PCR technology. See particularly, Mullis et al., Methods Enzymol. 155:335-350 (1987), and Higuchi, R. In PCR Technology: Principles and Applications for DNA Amplifications; Erlich, H. A., Ed.; Stockton Press Inc.: New York, 1989. Again, variants of these promoter fragments, such as those resulting from site-directed mutagenesis, are encompassed by the compositions of the present invention.

The term “target region” or “nucleic acid target” refers to a nucleotide sequence that resides at a specific chromosomal location. The “target region” or “nucleic acid target” is specifically recognized by a probe.

The term “total varin” means the combination of cannabinoids having propyl (three carbon) side chains, e.g, the combination of THCV+CBDV+CBCV+CBGV.

The term “transition” as used herein refers to the transition of a nucleotide at any specific genomic position with that of a different nucleotide.

The term “transgenic” refers to any cell, cell line, callus, tissue, plant part or plant, the genome of which has been altered by the presence of a heterologous nucleic acid, such as a recombinant DNA construct, including those initial transgenic events as well as those created by sexual crosses or asexual propagation from the initial transgenic event. The term “transgenic” as used herein does not encompass the alteration of the genome (chromosomal or extra-chromosomal) by conventional plant breeding methods or by naturally occurring events such as random cross-fertilization, non-recombinant viral infection, non-recombinant bacterial transformation, non-recombinant transposition, or spontaneous mutation. The term “transgenic plant” refers to a plant which comprises within its genome a heterologous polynucleotide. For example, the heterologous polynucleotide is stably integrated within the genome such that the polynucleotide is passed on to successive generations. The heterologous polynucleotide may be integrated into the genome alone or as part of a recombinant DNA construct.

The term “transformant” refers to a cell, tissue or organism that has undergone transformation. The original transformant is designated as “TO” or “TO.” Selfing the TO produces a first transformed generation designated as “T1” or “T1.”

The term “transformation” refers to the transfer of nucleic acid (i.e., a nucleotide polymer) into a cell. As used herein, the term “genetic transformation” refers to the transfer and incorporation of DNA, especially recombinant DNA, into a cell.

The term “transition” as used herein refers to the transition of a nucleotide at any specific genomic position with that of a different nucleotide.

The term “THCV” means tetrahydrocannabivarin.

The term “THCVA” means tetrahydrocannabivarinic acid.

The term “variety” as used herein has identical meaning to the corresponding definition in the International Convention for the Protection of New Varieties of Plants (UPOV treaty), of Dec. 2, 1961, as Revised at Geneva on Nov. 10, 1972, on Oct. 23, 1978, and on Mar. 19, 1991. Thus, “variety” means a plant grouping within a single botanical taxon of the lowest known rank, which grouping, irrespective of whether the conditions for the grant of a breeder's right are fully met, can be i) defined by the expression of the characteristics resulting from a given genotype or combination of genotypes, ii) distinguished from any other plant grouping by the expression of at least one of the said characteristics and iii) considered as a unit with regard to its suitability for being propagated unchanged.

The term “varin” refers to any cannabinoid having a unique propyl (3-carbon) side chain instead of a pentyl (5-carbon) side chain more commonly associated with cannabinoids.

The term “varin ratio” refers to [total varin/(total THC+total CBD+total CBC+total CBG)].

### Cannabis

Cannabis has long been used for drug and industrial purposes, fiber (hemp), for seed and seed oils, for medicinal purposes, and for recreational purposes. Industrial hemp products are made from Cannabis plants selected to produce an abundance of fiber. Some Cannabis varieties have been bred to produce minimal levels of THC, the principal psychoactive constituent responsible for the psychoactivity associated with marijuana. Marijuana has historically consisted of the dried flowers of Cannabis plants selectively bred to produce high levels of THC and other psychoactive cannabinoids. Various extracts including hashish and hash oil are also produced from the plant.

Cannabis is an annual, dioecious, flowering herb. The leaves are palmately compound or digitate, with serrate leaflets. Cannabis normally has imperfect flowers, with staminate “male” and pistillate “female” flowers occurring on separate plants. It is not unusual, however, for individual plants to separately bear both male and female flowers (i.e., have monoecious plants). Although monoecious plants are often referred to as “hermaphrodites,” true hermaphrodites (which are less common in Cannabis) bear staminate and pistillate structures on individual flowers, whereas monoecious plants bear male and female flowers at different locations on the same plant.

The life cycle of Cannabis varies with each variety but can be generally summarized into germination, vegetative growth, and reproductive stages. Because of heavy breeding and selection by humans, most Cannabis seeds have lost dormancy mechanisms and do not require any pre-treatments or winterization to induce germination (See Clarke, R C et al. “Cannabis: Evolution and Ethnobotany” University of California Press 2013). Seeds placed in viable growth conditions are expected to germinate in about 3 to 7 days. The first true leaves of a Cannabis plant contain a single leaflet, with subsequent leaves developing in opposite formation with increasing number of leaflets. Leaflets can be narrow or broad depending on the morphology of the plant grown. Cannabis plants are normally allowed to grow vegetatively for the first 4 to 8 weeks. During this period, the plant responds to increasing light with faster and faster growth. Under ideal conditions, Cannabis plants can grow up to 2.5 inches a day, and are capable of reaching heights of up to 20 feet. Indoor growth pruning techniques tend to limit Cannabis size through careful pruning of apical or side shoots.

Cannabis is diploid, having a chromosome complement of 2n=20, although polyploid individuals have been artificially produced. The first genome sequence of Cannabis, which is estimated to be 820 Mb in size, was published in 2011 by a team of Canadian scientists (Bakel et al, “The draft genome and transcriptome of Cannabis sativa” Genome Biology 12: R102).

All known varieties of Cannabis are wind-pollinated and the fruit is an achene. Most varieties of Cannabis are short day plants, with the possible exception of C. sativa subsp. sativa var. spontanea (=C. ruderalis), which is commonly described as “auto-flowering” and may be day-neutral.

The genus Cannabis was formerly placed in the Nettle (Urticaceae) or Mulberry (Moraceae) family, and later, along with the Humulus genus (hops), in a separate family, the Hemp family (Cannabaceae sensu stricto). Recent phylogenetic studies based on cpDNA restriction site analysis and gene sequencing strongly suggest that the Cannabaceae sensu stricto arose from within the former Celtidaceae family, and that the two families should be merged to form a single monophyletic family, the Cannabaceae sensu lato.

Cannabis plants produce a unique family of terpeno-phenolic compounds called cannabinoids. Cannabinoids, terpenoids, and other compounds are secreted by glandular trichomes that occur most abundantly on the floral calyxes and bracts of female plants. As a drug it usually comes in the form of dried flower buds (marijuana), resin (hashish), or various extracts collectively known as hashish oil. There are at least 483 identifiable chemical constituents known to exist in the Cannabis plant (Rudolf Brenneisen, 2007, Chemistry and Analysis of Phytocannabinoids (cannabinoids produced produced by Cannabis) and other Cannabis Constituents, In Marijuana and the Cannabinoids, ElSohly, ed.; incorporated herein by reference) and at least 85 different cannabinoids have been isolated from the plant (El-Alfy, Abir T, et al., 2010, “Antidepressant-like effect of delta-9-tetrahydrocannabinol and other cannabinoids isolated from Cannabis sativa L”, Pharmacology Biochemistry and Behavior 95 (4): 434-42; incorporated herein by reference). The two cannabinoids usually produced in greatest abundance are cannabidiol (CBD) and/or Δ9-tetrahydrocannabinol (THC). THC is psychoactive while CBD is not. See, ElSohly, ed. (Marijuana and the Cannabinoids, Humana Press Inc., 321 papers, 2007), which is incorporated herein by reference in its entirety, for a detailed description and literature review on the cannabinoids found in marijuana.

Cannabinoids are the most studied group of secondary metabolites in Cannabis. Most exist in two forms, as acids and in neutral (decarboxylated) forms. The acid form is designated by an “A” at the end of its acronym (i.e. THCA). The phytocannabinoids are synthesized in the plant as acid forms, and while some decarboxylation does occur in the plant, it increases significantly post-harvest and the kinetics increase at high temperatures. (Sanchez and Verpoorte 2008). The biologically active forms for human consumption are the neutral forms. Decarboxylation is usually achieved by thorough drying of the plant material followed by heating it, often by either combustion, vaporization, or heating or baking in an oven. Unless otherwise noted, references to cannabinoids in a plant include both the acidic and decarboxylated versions (e.g., CBD and CBDA).

Detection of neutral and acidic forms of cannabinoids are dependent on the detection method utilized. Two popular detection methods are high-performance liquid chromatography (HPLC) and gas chromatography (GC). HPLC separates, identifies, and quantifies different components in a mixture, and passes a pressurized liquid solvent containing the sample mixture through a column filled with a solid adsorbent material. Each molecular component in a sample mixture interacts differentially with the adsorbent material, thus causing different flow rates for the different components and therefore leading to separation of the components. In contrast, GC separates components of a sample through vaporization. The vaporization required for such separation occurs at high temperature. Thus, the main difference between GC and HPLC is that GC involves thermal stress and mainly resolves analytes by boiling points while HPLC does not involve heat and mainly resolves analytes by polarity. The consequence of utilizing different methods for cannabinoid detection therefore is that HPLC is more likely to detect acidic cannabinoid precursors, whereas GC is more likely to detect decarboxylated neutral cannabinoids.

The cannabinoids in Cannabis plants include, but are not limited to, Δ9-Tetrahydrocannabinol (Δ9-THC), Δ8-Tetrahydrocannabinol (Δ8-THC), Cannabichromene (CBC), Cannabicyclol (CBL), Cannabidiol (CBD), Cannabielsoin (CBE), Cannabigerol (CBG), Cannabinidiol (CBND), Cannabinol (CBN), Cannabitriol (CBT), and their propyl homologs, including, but are not limited to cannabidivarin (CBDV), Δ9-Tetrahydrocannabivarin (THCV), cannabichromevarin (CBCV), and cannabigerovarin (CBGV). See Holley et al. (Constituents of Cannabis sativa L. XI Cannabidiol and cannabichromene in samples of known geographical origin, J. Pharm. Sci. 64:892-894, 1975) and De Zeeuw et al. (Cannabinoids with a propyl side chain in Cannabis, Occurrence and chromatographic behavior, Science 175:778-779), each of which is herein incorporated by reference in its entirety for all purposes. Non-THC cannabinoids can be collectively referred to as “CBs”, wherein CBs can be one of THCV, CBDV, CBGV, CBCV, CBD, CBC, CBE, CBG, CBN, CBND, and CBT cannabinoids.

### Varin Markers and Haplotypes

Varins are a type of cannabinoid compounds having three carbon atoms in their alkyl side chain instead of the five carbon atom alkyl side chains more commonly associated with cannabinoids. Two such varins are tetrahydrocannabivarin (THCV) and cannabidivarin (CBDV), which are homologues of tetrahydrocannabinol (THC) and cannabidiol (CBD), respectively.

The present invention describes the discovery of causal markers indicating modified varin activity for plants, including Cannabis. Such markers can be used to allow for screening of plants exhibiting modified varin activity. For example, Tables 1 and 3, and SEQ ID NOs: 19-34 describe useful markers from different varin-related genes. Accordingly, the present invention describes a method for selecting one or more plants having elevated varin levels, the method comprising i) obtaining nucleic acids from a sample plant(s) or their germplasm; (ii) detecting one or more markers that indicate an elevated varin level phenotype, (iii) indicating the elevated varin level phenotype, and (iv) selecting the one or more plants indicating the elevated varin level phenotype. In an embodiment, the sample plant(s) is a progeny plant obtained from a cross between a first plant and a second plant wherein the first plant has elevated varin levels and the second plant either (a) does not have elevated varin levels, or (b) has elevated varin leves with progeny that do not segregate elevated varin levels.

*In an embodiment, the one or more markers comprises a polymorphism at position 51 of any one or more of SEQ ID NO:19; SEQ ID NO:20; SEQ ID NO:21; SEQ ID NO:22; SEQ ID NO: 23; SEQ ID NO:24; SEQ ID NO:25; SEQ ID NO:26; SEQ ID NO:27; SEQ ID NO:28; SEQ ID NO: 29; SEQ ID NO:30; SEQ ID NO:31; SEQ ID NO:32; SEQ ID NO:33; or SEQ ID NO:34. In an embodiment, the nucleotide position comprises an A/G or G/G genotype at position 51 of SEQ ID NO:19; a C/T or T/T genotype at position 51 of SEQ ID NO:20; a G/A or A/A genotype at position 51 of SEQ ID NO:21; a G/T or T/T genotype at position 51 of SEQ ID NO:22; a C/A or A/A genotype at position 51 of SEQ ID NO:23; a G/C or C/C genotype at position 51 of SEQ ID NO: 24; a G/T or T/T genotype at position 51 of SEQ ID NO:25; a G/A or A/A genotype at position 51 of SEQ ID NO:26; a G/A or A/A genotype at position 51 of SEQ ID NO:27; a G/A or A/A genotype at position 51 of SEQ ID NO:28; a T/C or C/C genotype at position 51 of SEQ ID NO: 29; a C/T or T/T genotype at position 51 of SEQ ID NO:30; a T/G or G/G genotype at position 51 of SEQ ID NO:31; an A/G or G/G genotype at position 51 of SEQ ID NO:32; an A/G or G/G genotype at position 51 of SEQ ID NO:33; or a G/A or A/A genotype at position 51 of SEQ ID NO:34.

Any marker existing with the causal gene described here, e.g., KR can include a polymorphic nucleotide that can be used as a marker for modified varin production, including the use of marker assisted selection as described herein

### Quantitative Trait Loci

The term chromosome interval designates a contiguous linear span of genomic DNA that resides on a single chromosome. A chromosome interval may comprise a quantitative trait locus (“QTL”) linked with a genetic trait and the QTL may comprise a single gene or multiple genes associated with the genetic trait. The boundaries of a chromosome interval comprising a QTL are drawn such that a marker that lies within the chromosome interval can be used as a marker for the genetic trait, as well as markers genetically linked thereto. Each interval comprising a QTL comprises at least one gene conferring a given trait, however knowledge of how many genes are in a particular interval is not necessary to make or practice the invention, as such an interval will segregate at meiosis as a linkage block. In accordance with the invention, a chromosomal interval comprising a QTL may therefore be readily introgressed and tracked in a given genetic background using the methods and compositions provided herein.

Identification of chromosomal intervals and QTL is therefore beneficial for detecting and tracking a genetic trait, such as modified varin activity, in plant populations. In some embodiments, this is accomplished by identification of markers linked to a particular QTL. The principles of QTL analysis and statistical methods for calculating linkage between markers and useful QTL include penalized regression analysis, ridge regression, single point marker analysis, complex pedigree analysis, Bayesian MCMC, identity-by-descent analysis, interval mapping, composite interval mapping (CIM), and Haseman-Elston regression. QTL analyses may be performed with the help of a computer and specialized software available from a variety of public and commercial sources known to those of skill in the art.

### Detection of Markers

Marker detection is well known in the art. For example, amplification of a target polynucleotide (e.g., by PCR) using a particular amplification primer pair that permit the primer pair to hybridize to the target polynucleotide to which a primer having the corresponding sequence (or its complement) would bind and preferably to produce an identifiable amplification product (the amplicon) having a marker is well known in the art.

Methods for designing PCR primers and PCR cloning are generally known in the art and are disclosed in Sambrook et al. (1989) Molecular Cloning: A Laboratory Manual (2d ed., Cold Spring Harbor Laboratory Press, Plainview, N.Y.). See also Innis et al., eds. (1990) PCR Protocols: A Guide to Methods and Applications (Academic Press, New York); Innis and Gelfand, eds. (1995) PCR Strategies (Academic Press, New York); and Innis and Gelfand, eds. (1999) PCR Methods Manual (Academic Press, New York). Methods of amplification are further described in U.S. Pat. Nos. 4,683,195, 4,683,202 and Chen et al. (1994) PNAS 91:5695-5699. These methods as well as other methods known in the art of DNA amplification may be used in the practice of the embodiments of the present invention. It will be appreciated that suitable primers to be used with the invention can be designed using any suitable method. It is not intended that the invention be limited to any particular primer or primer pair. It is not intended that the primers of the invention be limited to generating an amplicon of any particular size. For example, the primers used to amplify the marker loci and alleles herein are not limited to amplifying the entire region of the relevant locus. The primers can generate an amplicon of any suitable length that is longer or shorter than those disclosed herein. In some embodiments, marker amplification produces an amplicon at least 20 nucleotides in length, or alternatively, at least 50 nucleotides in length, or alternatively, at least 100 nucleotides in length, or alternatively, at least 200 nucleotides in length. It is understood that a number of parameters in a specific PCR protocol may need to be adjusted to specific laboratory conditions and may be slightly modified and yet allow for the collection of similar results. The primers of the invention may be radiolabeled, or labeled by any suitable means (e.g., using a non-radioactive fluorescent tag), to allow for rapid visualization of the different size amplicons following an amplification reaction without any additional labeling step or visualization step. The known nucleic acid sequences for the genes described herein are sufficient to enable one of skill in the art to routinely select primers for amplification of the gene of interest.

Other suitable amplification methods include, but are not limited to, ligase chain reaction (LCR) (see, Wu and Wallace (1989) Genomics 4:560, Landegren et al. (1988) Science 241:1077, and Barringer et al. (1990) Gene 89:117), transcription amplification (Kwoh et al. (1989) Proc. Natl. Acad. Sci. USA 86:1173), self-sustained sequence replication (Guatelli et al. (1990) Proc. Natl. Acad. Sci. USA 87:1874), dot PCR, and linker adapter PCR, etc.

An amplicon is an amplified nucleic acid, e.g., a nucleic acid that is produced by amplifying a template nucleic acid by any available amplification method (e.g., PCR, LCR, transcription, or the like). A genomic nucleic acid is a nucleic acid that corresponds in sequence to a heritable nucleic acid in a cell. Common examples include nuclear genomic DNA and amplicons thereof. A genomic nucleic acid is, in some cases, different from a spliced RNA, or a corresponding cDNA, in that the spliced RNA or cDNA is processed, e.g., by the splicing machinery, to remove introns. Genomic nucleic acids optionally comprise non-transcribed (e.g., chromosome structural sequences, promoter regions, enhancer regions, etc.) and/or non-translated sequences (e.g., introns), whereas spliced RNA/cDNA typically do not have non-transcribed sequences or introns. A template nucleic acid is a nucleic acid that serves as a template in an amplification reaction (e.g., a polymerase based amplification reaction such as PCR, a ligase mediated amplification reaction such as LOR, a transcription reaction, or the like). A template nucleic acid can be genomic in origin, or alternatively, can be derived from expressed sequences, e.g., a cDNA or an EST. Details regarding the use of these and other amplification methods can be found in any of a variety of standard texts. Many available biology texts also have extended discussions regarding PCR and related amplification methods and one of skill will appreciate that essentially any RNA can be converted into a double stranded DNA suitable for restriction digestion, PCR expansion and sequencing using reverse transcriptase and a polymerase.

PCR detection and quantification using dual-labeled fluorogenic oligonucleotide probes, commonly referred to as “TaqMan™” probes, can also be performed according to the present invention. These probes are composed of short (e.g., 20-25 base) oligodeoxynucleotides that are labeled with two different fluorescent dyes. On the 5′ terminus of each probe is a reporter dye, and on the 3′ terminus of each probe a quenching dye is found. The oligonucleotide probe sequence is complementary to an internal target sequence present in a PCR amplicon. When the probe is intact, energy transfer occurs between the two fluorophores and emission from the reporter is quenched by the quencher by FRET. During the extension phase of PCR, the probe is cleaved by 5′ nuclease activity of the polymerase used in the reaction, thereby releasing the reporter from the oligonucleotide-quencher and producing an increase in reporter emission intensity. TaqMan™ probes are oligonucleotides that have a label and a quencher, where the label is released during amplification by the exonuclease action of the polymerase used in amplification, providing a real time measure of amplification during synthesis. A variety of TaqMan™ reagents are commercially available, e.g., from Applied Biosystems as well as from a variety of specialty vendors such as Biosearch Technologies.

In general, synthetic methods for making oligonucleotides, including probes, primers, molecular beacons, PNAs, LNAs (locked nucleic acids), etc., are well known. For example, oligonucleotides can be synthesized chemically according to the solid phase phosphoramidite triester method described. Oligonucleotides, including modified oligonucleotides, can also be ordered from a variety of commercial sources.

Nucleic acid probes to the marker loci can be cloned and/or synthesized. Any suitable label can be used with a probe of the invention. Detectable labels suitable for use with nucleic acid probes include, for example, any composition detectable by spectroscopic, radioisotopic, photochemical, biochemical, immunochemical, electrical, optical or chemical means. Useful labels include biotin for staining with labeled streptavidin conjugate, magnetic beads, fluorescent dyes, radio labels, enzymes, and colorimetric labels. Other labels include ligands which bind to antibodies labeled with fluorophores, chemiluminescent agents, and enzymes. A probe can also constitute radio labeled POR primers that are used to generate a radio labeled amplicon. It is not intended that the nucleic acid probes of the invention be limited to any particular size.

Amplification is not always a requirement for marker detection (e.g. Southern blotting and RFLP detection). Separate detection probes can also be omitted in amplification/detection methods, e.g., by performing a real time amplification reaction that detects product formation by modification of the relevant amplification primer upon incorporation into a product, incorporation of labeled nucleotides into an amplicon, or by monitoring changes in molecular rotation properties of amplicons as compared to unamplified precursors (e.g., by fluorescence polarization).

### Varin Genes

The QTL data as described herein identifies the KR gene (SEQ ID NO: 1 and SEQ ID NO: 2 (protein)) as possibly involved in the production of modified varin activity in Cannabis. The KR gene pt/mtKR/FabG1 (β ketoacyl-acyl carrier protein (ACP) reductase, At1g24360) is also referred to as 3-oxoacyl-[acyl-carrier-protein] reductase. KR functions together with enoyl-ACP reductase (pt/mtER) to catalyze two of the reactions that constitute the core four-reaction cycle of the fatty acid biosynthesis (FAS) system, which iteratively elongates the acyl-chain by two carbon atoms per cycle (Guan et al. 2020, Plant Physiology 183 (2): 517-529). In Cannabis, plastid fatty acid biosynthesis forms the precursor for the acyl chain in THC and THCV (Welling et al. 2019, Scientific Reports 9(1): 1-13). Allelic variation of KR likely produces a KR variant that results in a shorter propyl (3-carbon) side chain found in THCV instead of a pentyl (5-carbon) group found in THC. Guan et al. (2020, Plant Physiology 183 (2): 517-529) describe a single copy of KR in Arabidopsis, the presence of a pre-sequence determines whether it localizes to the chloroplast, where it is involved in fatty acid chain elongation, or to the mitochondria where it is involved in different processes. A T-DNA insertion in KR in Arabidopsis makes it embryonic lethal, further supporting the notion that it is essential for fatty acid biosynthesis. In Cannabis there appear to be at least 3 copies of KR with varying expression levels in stalked capitate trichomes between the low Total Varin (0.11%) variety ‘Purple Kush’ and the intermediate Total Varin variety ‘Finola’ (0.74%; Livingston et al. 2020, The Plant Journal 101:37-56). BLASTP results of the mapped ‘Abacus’ KR sequence on CBDRx reference genome result in: 3-oxoacyl-[acyl-carrier-protein] reductase 4 [Cannabis sativa] 100% protein sequence match for the full 322 AA sequence. Gene expression analysis of Cannabis stalked capitate trichomes identifies 3 KR genes with homologs in Brassica napus: fabg1, fabg2, and fabg3. Two of these genes, fabg1 and fabg3 display significant expression differences between ‘Finola’ and ‘Purple Kush’ (Livingston et al. 2020, The Plant Journal 101:37-56). BLASTN of the transcriptome fragments of these three genes identified fabg1 in ‘Finola’ and ‘Purple Kush’ as the homolog of the mapped ‘Abacus’ KR gene.

Table 4 provides the sequence listings of causal polymorphisms conferring modified varin activity. In particular, SEQ ID NOS: 19-34 describe specific polymorphic variations in the KR gene leading to modified varin activity, and SEQ ID NOs: 35-50 describe the corresponding amino acid sequence of KR protein products responsible for modified varin activity.* Example 3 describes the elevated varin levels or increased varin ratios achieved by said polymorphic variations.

### Cannabis Breeding

Cannabis is an important and valuable crop. Thus, a continuing goal of Cannabis plant breeders is to develop stable, high yielding Cannabis cultivars that are agronomically sound. To accomplish this goal, the Cannabis breeder preferably selects and develops Cannabis plants with traits that result in superior cultivars. The plants described herein can be used to produce new plant varieties. In some embodiments, the plants are used to develop new, unique, and superior varieties or hybrids with desired phenotypes.

The development of commercial Cannabis cultivars requires the development of Cannabis varieties, the crossing of these varieties, and the evaluation of the crosses. Pedigree breeding and recurrent selection breeding methods may be used to develop cultivars from breeding populations. Breeding programs may combine desirable traits from two or more varieties or various broad-based sources into breeding pools from which cultivars are developed by selfing and selection of desired phenotypes. The new cultivars may be crossed with other varieties and the hybrids from these crosses are evaluated to determine which have commercial potential.

Details of existing Cannabis plants varieties and breeding methods are described in Potter et al. (2011, World Wide Weed: Global Trends in Cannabis Cultivation and Its Control), Holland (2010, The Pot Book: A Complete Guide to Cannabis, Inner Traditions/Bear & Co, ISBN1594778981, 9781594778988), Green I (2009, The Cannabis Grow Bible: The Definitive Guide to Growing Marijuana for Recreational and Medical Use, Green Candy Press, 2009, ISBN 1931160589, 9781931160582), Green II (2005, The Cannabis Breeder's Bible: The Definitive Guide to Marijuana Genetics, Cannabis Botany and Creating Strains for the Seed Market, Green Candy Press, 1931160279, 9781931160278), Starks (1990, Marijuana Chemistry: Genetics, Processing & Potency, ISBN 0914171399, 9780914171393), Clarke (1981, Marijuana Botany, an Advanced Study: The Propagation and Breeding of Distinctive Cannabis, Ronin Publishing, ISBN 091417178X, 9780914171782), Short (2004, Cultivating Exceptional Cannabiss: An Expert Breeder Shares His Secrets, ISBN 1936807122, 9781936807123), Cervantes (2004, Marijuana Horticulture: The Indoor/Outdoor Medical Grower's Bible, Van Patten Publishing, ISBN 187882323X, 9781878823236), Franck et al. (1990, Marijuana Grower's Guide, Red Eye Press, ISBN 0929349016, 9780929349015), Grotenhermen and Russo (2002, Cannabis and Cannabinoids: Pharmacology, Toxicology, and Therapeutic Potential, Psychology Press, ISBN 0789015080, 9780789015082), Rosenthal (2007, The Big Book of Buds: More Marijuana Varieties from the World's Great Seed Breeders, ISBN 1936807068, 9781936807062), Clarke, RC (Cannabis: Evolution and Ethnobotany 2013 (In press)), King, J (Cannabible Vols 1-3, 2001-2006), and four volumes of Rosenthal's Big Book of Buds series (2001, 2004, 2007, and 2011), each of which is herein incorporated by reference in its entirety for all purposes.

Pedigree selection, where both single plant selection and mass selection practices are employed, may be used for the generating varieties as described herein. Pedigree selection, also known as the “Vilmorin system of selection,” is described in Fehr, Walter; Principles of Cultivar Development, Volume I, Macmillan Publishing Co., which is hereby incorporated by reference. Pedigree breeding is used commonly for the improvement of self-pollinating crops or inbred lines of cross-pollinating crops. Two parents which possess favorable, complementary traits are crossed to produce an F1. An F2 population is produced by selfing one or several F1's or by intercrossing two F1's (sib mating). Selection of the best individuals usually begins in the F2 population; then, beginning in the F3, the best individuals in the best families are usually selected. Replicated testing of families, or hybrid combinations involving individuals of these families, often follows in the F4 generation to improve the effectiveness of selection for traits with low heritability. At an advanced stage of inbreeding (e.g., F6 and F7), the best lines or mixtures of phenotypically similar lines are tested for potential release as new cultivars.

Choice of breeding or selection methods depends on the mode of plant reproduction, the heritability of the trait(s) being improved, and the type of cultivar used commercially (e.g., F1 hybrid cultivar, pureline cultivar, etc.). For highly heritable traits, a choice of superior individual plants evaluated at a single location will be effective, whereas for traits with low heritability, selection should be based on mean values obtained from replicated evaluations of families of related plants. Popular selection methods commonly include pedigree selection, modified pedigree selection, mass selection, and recurrent selection.

Mass and recurrent selections can be used to improve populations of either self- or cross-pollinating crops. A genetically variable population of heterozygous individuals may be identified or created by intercrossing several different parents. The best plants may be selected based on individual superiority, outstanding progeny, or excellent combining ability. Preferably, the selected plants are intercrossed to produce a new population in which further cycles of selection are continued.

Backcross breeding has been used to transfer genes for a simply inherited, highly heritable trait into a desirable homozygous cultivar or line that is the recurrent parent. The source of the trait to be transferred is called the donor parent. The resulting plant is expected to have the attributes of the recurrent parent (e.g., cultivar) and the desirable trait transferred from the donor parent. After the initial cross, individuals possessing the phenotype of the donor parent may be selected and repeatedly crossed (backcrossed) to the recurrent parent. The resulting plant is expected to have the attributes of the recurrent parent (e.g., cultivar) and the desirable trait transferred from the donor parent.

A single-seed descent procedure refers to planting a segregating population, harvesting a sample of one seed per plant, and using the one-seed sample to plant the next generation. When the population has advanced from the F2 to the desired level of inbreeding, the plants from which lines are derived will each trace to different F₂ individuals. The number of plants in a population declines each generation due to failure of some seeds to germinate or some plants to produce at least one seed. As a result, not all of the F2 plants originally sampled in the population will be represented by a progeny when generation advance is completed.

Mutation breeding is another method of introducing new traits into Cannabis varieties. Mutations that occur spontaneously or are artificially induced can be useful sources of variability for a plant breeder. The goal of artificial mutagenesis is to increase the rate of mutation for a desired characteristic. Mutation rates can be increased by many different means including temperature, long-term seed storage, tissue culture conditions, radiation (such as X-rays, Gamma rays, neutrons, Beta radiation, or ultraviolet radiation), chemical mutagens (such as base analogs like 5-bromo-uracil), antibiotics, alkylating agents (such as sulfur mustards, nitrogen mustards, epoxides, ethyleneamines, sulfates, sulfonates, sulfones, or lactones), azide, hydroxylamine, nitrous acid or acridines. Once a desired trait is observed through mutagenesis the trait may then be incorporated into existing germplasm by traditional breeding techniques. Details of mutation breeding can be found in Principles of Cultivar Development by Fehr, Macmillan Publishing Company, 1993.

The complexity of inheritance also influences the choice of the breeding method. Backcross breeding may be used to transfer one or a few favorable genes for a highly heritable trait into a desirable cultivar. This approach has been used extensively for breeding disease-resistant cultivars. Various recurrent selection techniques are used to improve quantitatively inherited traits controlled by numerous genes. The use of recurrent selection in self-pollinating crops depends on the ease of pollination, the frequency of successful hybrids from each pollination, and the number of hybrid offspring from each successful cross.

Additional breeding methods have been known to one of ordinary skill in the art, e.g., methods discussed in Chahal and Gosal (Principles and procedures of plant breeding: biotechnological and conventional approaches, CRC Press, 2002, ISBN 084931321X, 9780849313219), Taji et al. (In vitro plant breeding, Routledge, 2002, ISBN 156022908X, 9781560229087), Richards (Plant breeding systems, Taylor & Francis US, 1997, ISBN 0412574500, 9780412574504), Hayes (Methods of Plant Breeding, Publisher: READ BOOKS, 2007, ISBN1406737062, 9781406737066), each of which is incorporated by reference in its entirety for all purposes. Cannabis genome has been sequenced (Bakel et al., The draft genome and transcriptome of Cannabis sativa, Genome Biology, 12 (10): R102, 2011). Molecular markers for Cannabis plants are described in Datwyler et al. (Genetic variation in hemp and marijuana (Cannabis sativa L.) according to amplified fragment length polymorphisms, J Forensic Sci. 2006 March; 51 (2): 371-5), Pinarkara et al., (RAPD analysis of seized marijuana (Cannabis sativa L.) in Turkey, Electronic Journal of Biotechnology, 12 (1), 2009), Hakki et al., (Inter simple sequence repeats separate efficiently hemp from marijuana (Cannabis sativa L.), Electronic Journal of Biotechnology, 10 (4), 2007), Datwyler et al., (Genetic Variation in Hemp and Marijuana (Cannabis sativa L.) According to Amplified Fragment Length Polymorphisms, J Forensic Sci, March 2006, 51 (2): 371-375), Gilmore et al. (Isolation of microsatellite markers in Cannabis sativa L. (marijuana), Molecular Ecology Notes, 3 (1): 105-107, March 2003), Pacifico et al., (Genetics and marker-assisted selection of chemotype in Cannabis sativa L.), Molecular Breeding (2006) 17:257-268), and Mendoza et al., (Genetic individualization of Cannabis sativa by a short tandem repeat multiplex system, Anal Bioanal Chem (2009) 393:719-726), each of which is herein incorporated by reference in its entirety for all purposes.

The production of double haploids can also be used for the development of homozygous varieties in a breeding program. Double haploids are produced by the doubling of a set of chromosomes from a heterozygous plant to produce a completely homozygous individual. For example, see Wan et al., Theor. Appl. Genet., 77:889-892, 1989.

### Marker Assisted Selection Breeding

In an embodiment, marker assisted selection (MAS) is used to produce plants with desired traits. MAS is a powerful shortcut to selecting for desired phenotypes and for introgressing desired traits into cultivars (e.g., introgressing desired traits into elite lines). MAS is easily adapted to high throughput molecular analysis methods that can quickly screen large numbers of plant or germplasm genetic material for the markers of interest and is much more cost effective than raising and observing plants for visible traits.

In some embodiments, the invention therefore provides quantitative trait loci (QTL) that demonstrate significant co-segregation with elevated THCV, THCVA, CBDV, CBDVA, CBGV, or CBGVA levels. The QTL of the invention can be tracked during plant breeding or introgressed into a desired genetic background in order to provide novel plants exhibiting elevated THCV, THCVA, CBDV, CBDVA, CBGV, or CBGVA levels and one or more other beneficial traits. Molecular markers linked to the QTL of the invention and methods of using the markers for detection of and selection for elevated THCV, THCVA, CBDV, CBDVA, CBGV, or CBGVA levels can be used. Thus, embodiments of the invention therefore include specific markers, chromosome intervals comprising the markers, and methods of detecting markers genetically linked to elevated THCV, THCVA, CBDV, CBDVA, CBGV, or CBGVA levels. Also provided herein are markers that are useful for detecting the presence or absence of THCV, THCVA, CBDV, CBDVA, CBGV, or CBGVA activity alleles within the QTL of the invention that can be used in marker assisted selection (MAS) breeding programs to produce plants with a desired elevated THCV, THCVA, CBDV, CBDVA, CBGV, or CBGVA levels. Also provided herein are markers that are useful for detecting the presence or absence of THCV, THCVA, CBDV, CBDVA, CBGV, or CBGVA activity alleles within the QTL of the invention that can be used in marker assisted selection (MAS) breeding programs to produce plants with a desired level of THCV, THCVA, CBDV, CBDVA, CBGV, or CBGVA.

The invention further provides methods of using the markers identified herein to introgress loci associated with high THCV, THCVA, CBDV, CBDVA, CBGV, or CBGVA levels into plants. Thus, one skilled in the art can use the invention to create novel Cannabis plants with elevated THCV, THCVA, CBDV, CBDVA, CBGV, or CBGVA activity by crossing a donor line comprising a QTL associated with THCV, THCVA, CBDV, CBDVA, CBGV, or CBGVA activity into any desired recipient line, with or without MAS. Resulting progeny can be selected to be genetically similar to the recipient line except for the THCV, THCVA, CBDV, CBDVA, CBGV, or CBGVA activity QTL.

Introgression refers to the transmission of a desired allele of a genetic locus from one genetic background to another, which is significantly assisted through MAS. For example, introgression of a desired allele at a specified locus can be transmitted to at least one progeny via a sexual cross between two parents of the same species, where at least one of the parents has the desired allele in its genome. Alternatively, for example, transmission of an allele can occur by recombination between two donor genomes, e.g., in a fused protoplast, where at least one of the donor protoplasts has the desired allele in its genome. The desired allele can be, e.g., a selected allele of a marker, a QTL, a transgene, or the like.

The introgression of one or more desired loci from a donor line into another is achieved via repeated backcrossing to a recurrent parent accompanied by selection to retain one or more loci from the donor parent. Markers associated with varin activity may be assayed in progeny and those progeny with one or more desired markers are selected for advancement. In another aspect, one or more markers can be assayed in the progeny to select for plants with the genotype of the agronomically elite parent. This invention anticipates that trait introgressed varin modification will require more than one generation, wherein progeny are crossed to the recurrent (agronomically elite) parent or selfed. Selections are made based on the presence of one or more varin markers and can also be made based on the recurrent parent genotype, wherein screening is performed on a genetic marker and/or phenotype basis. In another embodiment, markers of this invention can be used in conjunction with other markers, ideally at least one on each chromosome of the Cannabis genome, to track the modified varin activity phenotypes.

Genetic markers are used to identify plants that contain a desired genotype at one or more loci, and that are expected to transfer the desired genotype, along with a desired phenotype to their progeny. Genetic markers can be used to identify plants containing a desired genotype at one locus, or at several unlinked or linked loci (e.g., a haplotype), and that would be expected to transfer the desired genotype, along with a desired phenotype to their progeny. The present invention provides the means to identify plants that exhibit a modified varin phenotype by identifying plants having varin-specific markers.

In general, MAS uses polymorphic markers that have been identified as having a significant likelihood of co-segregation with a desired trait. Such markers are presumed to map near a gene or genes that give the plant its desired phenotype, and are considered indicators for the desired trait, and are termed QTL markers. Plants are tested for the presence or absence of a desired allele in the QTL marker.

Identification of plants or germplasm that include a marker locus or marker loci linked to a desired trait or traits provides a basis for performing MAS. Plants that comprise favorable markers or favorable alleles are selected for, while plants that comprise markers or alleles that are negatively correlated with the desired trait can be selected against. Desired markers and/or alleles can be introgressed into plants having a desired (e.g., elite or exotic) genetic background to produce an introgressed plant or germplasm having the desired trait. In some aspects, it is contemplated that a plurality of markers for desired traits are sequentially or simultaneously selected and/or introgressed. The combinations of markers that are selected for in a single plant is not limited and can include any combination of markers disclosed herein or any marker linked to the markers disclosed herein, or any markers located within the QTL intervals defined herein.

In some embodiments, a first Cannabis plant or germplasm exhibiting a desired trait (the donor) can be crossed with a second Cannabis plant or germplasm (the recipient, e.g., an elite or exotic Cannabis, depending on characteristics that are desired in the progeny) to create an introgressed Cannabis plant or germplasm as part of a breeding program. In some aspects, the recipient plant can also contain one or more loci associated with one or more desired traits, which can be qualitative or quantitative trait loci. In another aspect, the recipient plant can contain a transgene.

MAS, as described herein, using additional markers flanking either side of the DNA locus provide further efficiency because an unlikely double recombination event would be needed to simultaneously break linkage between the locus and both markers. Moreover, using markers tightly flanking a locus, one skilled in the art of MAS can reduce linkage drag by more accurately selecting individuals that have less of the potentially deleterious donor parent DNA. Any marker linked to or among the chromosome intervals described herein can thus find use within the scope of this invention.

Similarly, by identifying plants lacking a desired marker locus, plants having low varin activity can be identified and eliminated from subsequent crosses. These marker loci can be introgressed into any desired genomic background, germplasm, plant, line, variety, etc., as part of an overall MAS breeding program designed to modify varin activity. The invention also provides chromosome QTL intervals that can be used in MAS to select plants that demonstrate different varin traits. The QTL intervals can also be used to counter-select plants that do not exhibit increased varin activity.

Thus, the invention permits one skilled in the art to detect the presence or absence of varin modification genotypes in the genomes of Cannabis plants as part of a MAS program, as described herein. In one embodiment, a breeder ascertains the genotype at one or more markers for a parent having favorable varin modification activity, which contains a favorable varin modification activity allele, and the genotype at one or more markers for a parent with unfavorable varin modification activity, which lacks the favorable varin modification activity allele. A breeder can then reliably track the inheritance of the varin modification activity alleles through subsequent populations derived from crosses between the two parents by genotyping offspring with the markers used on the parents and comparing the genotypes at those markers with those of the parents. Depending on how tightly linked the marker alleles are with the trait, progeny that share genotypes with the parent having varin modification activity alleles can be reliably predicted to express the desirable phenotype and progeny that share genotypes with the parent having unfavorable varin modification activity alleles can be reliably predicted to express the undesirable phenotype. Thus, the laborious, inefficient, and potentially inaccurate process of manually phenotyping the progeny for varin modification activity traits is avoided.

Closely linked markers flanking the locus of interest that have alleles in linkage disequilibrium with varin modification activity alleles at that locus may be effectively used to select for progeny plants with desirable varin modification activity traits. Thus, the markers described herein, as well as other markers genetically linked to the same chromosome interval, may be used to select for Cannabis plants with different varin modification activity traits. Often, a haplotype, which is a set of these markers will be used, (e.g., 2 or more, 3 or more, 4 or more, 5 or more) in the flanking regions of the locus. Optionally, as described above, a marker flanking or within the actual locus may also be used. The parents and their progeny may be screened for these sets of markers, and the markers that are polymorphic between the two parents used for selection. In an introgression program, this allows for selection of the gene or locus genotype at the more proximal polymorphic markers and selection for the recurrent parent genotype at the more distal polymorphic markers.

In an embodiment, MAS is used to select one or more Cannabis plants comprising varin modification activity, the method comprising: (i) obtaining nucleic acids from the sample Cannabis plant or germplasm; (ii) detecting one or more markers that indicate varin modification activity, (iii) indicating varin modification activity, and (iv) selecting the one or more plants indicating the varin modification activity.

A number of SNPs together within a sequence, or across linked sequences, can be used to describe a haplotype for any particular genotype (Ching et al. (2002), BMC Genet. 3:19 pp Gupta et al. 2001, Rafalski (2002b), Plant Science 162:329-333). Haplotypes may in some circumstances be more informative than single SNPs and can be more descriptive of any particular genotype.

The choice of markers actually used to practice the invention is not limited and can be any marker that is genetically linked to the intervals as described herein, which includes markers mapping within the intervals. In certain embodiments, the invention further provides markers closely genetically linked to, or within approximately 0.5 CM of, the markers provided herein and chromosome intervals whose borders fall between or include such markers, and including markers within approximately 0.4 cM, 0.3 cM, 0.2 cM, and about 0.1 cM of the markers provided herein.

In some embodiments the markers and haplotypes described above can be used for marker assisted selection to produce additional progeny plants comprising the indicated varin modification activity. In some embodiments, backcrossing may be used in conjunction with marker-assisted selection.

### Gene Editing

In some embodiments gene editing is used to develop plants having modified varin activity. In some embodiments as described herein, a transgenic Cannabis plant whose genome comprises one or more amino acid substitutions of at least a portion of an endogenous KR gene and wherein the Cannabis plant comprises elevated varin levels. In some embodiments the endogenous KR gene of the transgenic Cannabis plant comprises a genomic nucleic acid sequence having at least 90% sequence identity to SEQ ID NO:14 or a protein coding amino acid sequence having at least 90% sequence identity to SEQ ID NO:17. In some embodiments, SEQ ID NOS: 19-34 describe specific polymorphic variations of the transgenic Cannabis plants, and in other embodiments, SEQ ID NOs: 35-50 describe the corresponding amino acid sequence of KR protein products of the transgenic Cannabis plants.* In some embodiments, as described in Table 4, transgenic plants that are heterozygous for beneficial polymorphisms identified in the KR gene confer increases in varin production. In other embodiments, a cell isolated from the transgenic Cannabis plant is provided. In other embodiments, a Cannabis product made from the transgenic Cannabis plant is provided. In some embodiments, the modified varin is described as elevated total varin. In some embodiments, the modified varin is described as an increased varin ratio.

In other embodiments, an isolated nucleic acid sequence encoding one or more amino acid substitutions of at least a portion of an endogenous Cannabis KR gene is provided. In some embodiments, the isolated nucleic acid sequence of claim 11 wherein the endogenous Cannabis KR gene comprises a genomic nucleic acid sequence having at least 90% sequence identity to SEQ ID NO: 14 or a protein coding amino acid sequence having at least 90% sequence identity to SEQ ID NO:17. In some embodiments, SEQ ID NOS: 19-34 describe specific polymorphic variations of the isolated nucleic acid sequence, and in other embodiments, SEQ ID NOs: 35-50 describe the corresponding amino acid sequence of KR protein products of the isolated nucleic acid sequence.*

In other embodiments, methods of making a Cannabis plant with elevated varin levels is provided. In some embodiments, the method comprises replacing a nucleotide present within an endogenous KR gene with the isolated nucleic acid of claim 11. In some embodiments, SEQ ID NOS: 19-34 describe specific polymorphic variations of the methods of making a Cannabis plant with elevated varin levels, and in other embodiments, SEQ ID NOs: 35-50 describe the corresponding amino acid sequence of KR protein products of the methods of making a Cannabis plant with elevated varin levels.* In some embodiments, the modified varin is described as elevated total varin. In some embodiments, the modified varin is described as an increased varin ratio.

Preferred substantially similar nucleic acid sequences encompassed by this invention are those sequences that are 80% identical to the nucleic acid fragments reported herein or which are 80% identical to any portion of the nucleotide sequences reported herein. More preferred are nucleic acid fragments which are 90% identical to the nucleic acid sequences reported herein, or which are 90% identical to any portion of the nucleotide sequences reported herein. Most preferred are nucleic acid fragments which are 95% identical to the nucleic acid sequences reported herein, or which are 95% identical to any portion of the nucleotide sequences reported herein. It is well understood by one skilled in the art that many levels of sequence identity are useful in identifying related polynucleotide sequences. Useful examples of percent identities are those listed above, or also preferred is any integer percentage from 72% to 100%, such as 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% and 100%.

In an embodiment, an isolated polynucleotide is provided comprising a nucleotide sequence having at least 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% and 100% sequence identity compared to the claimed sequence, based on the Clustal V method of alignment with pairwise alignment default parameters (KTUPLE=2, GAP PENALTY=5, WINDOW=4 and DIAGONALS SAVED=4).

Local sequence alignment programs are similar in their calculation, but only compare aligned fragments of the sequences rather than utilizing an end-to-end analysis. Local sequence alignment programs such as BLAST® can be used to compare specific regions of two sequences. A BLAST® comparison of two sequences results in an E-value, or expectation value, that represents the number of different alignments with scores equivalent to or better than the raw alignment score, S, that are expected to occur in a database search by chance. The lower the E value, the more significant the match. Because database size is an element in E-value calculations, E-values obtained by BLASTing against public databases, such as GENBANK, have generally increased over time for any given query/entry match. In setting criteria for confidence of polypeptide function prediction, a “high” BLAST® match is considered herein as having an E-value for the top BLAST® hit of less than 1E-30; a medium BLASTX E-value is 1E-30 to 1E-8; and a low BLASTX E-value is greater than 1E-8. The protein function assignment in the present invention is determined using combinations of E-values, percent identity, query coverage and hit coverage. Query coverage refers to the percent of the query sequence that is represented in the BLAST® alignment. Hit coverage refers to the percent of the database entry that is represented in the BLAST® alignment. In one embodiment of the invention, function of a query polypeptide is inferred from function of a protein homolog where either (1) hit_p<1e-30 or % identity >35% AND query_coverage >50% AND hit_coverage >50%, or (2) hit_p<1e-8 AND query_coverage >70% AND hit_coverage >70%. The following abbreviations are produced during a BLAST® analysis of a sequence. SEQ_NUM provides the SEQ ID NO for the listed recombinant polynucleotide sequences. CONTIG_ID provides an arbitrary sequence name taken from the name of the clone from which the cDNA sequence was obtained. PROTEIN_NUM provides the SEQ ID NO for the recombinant polypeptide sequence NCBI_GI provides the GenBank ID number for the top BLAST® hit for the sequence. The top BLAST® hit is indicated by the National Center for Biotechnology Information GenBank Identifier number. NCBI_GI_DESCRIPTION refers to the description of the GenBank top BLAST® hit for sequence. E_VALUE provides the expectation value for the top BLAST® match. MATCH_LENGTH provides the length of the sequence which is aligned in the top BLAST® match TOP_HIT_PCT_IDENT refers to the percentage of identically matched nucleotides (or residues) that exist along the length of that portion of the sequences which is aligned in the top BLAST® match. CAT_TYPE indicates the classification scheme used to classify the sequence. GO_BP=Gene Ontology Consortium-biological process; GO_CC=Gene Ontology Consortium—cellular component; GO_MF=Gene Ontology Consortium molecular function; KEGG=KEGG functional hierarchy (KEGG=Kyoto Encyclopedia of Genes and Genomes); EC=Enzyme Classification from ENZYME data bank release 25.0; POI=Pathways of Interest. CAT_DESC provides the classification scheme subcategory to which the query sequence was assigned. PRODUCT_CAT_DESC provides the FunCAT annotation category to which the query sequence was assigned. PRODUCT_HIT_DESC provides the description of the BLAST® hit which resulted in assignment of the sequence to the function category provided in the cat_desc column. HIT_E provides the E value for the BLAST® hit in the hit_desc column. PCT_IDENT refers to the percentage of identically matched nucleotides (or residues) that exist along the length of that portion of the sequences which is aligned in the BLAST® match provided in hit_desc. QRY_RANGE lists the range of the query sequence aligned with the hit. HIT RANGE lists the range of the hit sequence aligned with the query. provides the percent of query sequence length that matches QRY_CVRG provides the percent of query sequence length that matches to the hit (NCBI) sequence in the BLAST® match (% qry cvrg=(match length/query total length)×100). HIT_CVRG provides the percent of hit sequence length that matches to the query sequence in the match generated using BLAST® (% hit cvrg=(match lengthy hit total length)×100).

Methods for aligning sequences for comparison are well-known in the art. Various programs and alignment algorithms are described. In an embodiment, the subject disclosure relates to calculating percent identity between two polynucleotides or amino acid sequences using an AlignX alignment program of the Vector NTI suite (Invitrogen, Carlsbad, Calif.). The AlignX alignment program is a global sequence alignment program for polynucleotides or proteins. In an embodiment, the subject disclosure relates to calculating percent identity between two polynucleotides or amino acid sequences using the MegAlign program of the LASERGENE bioinformatics computing suite (MegAlign™ (.COPYRGT.1993-2016).

DNASTAR. Madison, Wis.). The MegAlign program is a global sequence alignment program for polynucleotides or proteins.

Gene editing is well known in the art, and many methods can be used with the present invention. For example, a skilled artisan will recognize that the ability to engineer a trait relies on the action of the genome editing proteins and various endogenous DNA repair pathways. These pathways may be normally present in a cell or may be induced by the action of the genome editing protein. Using genetic and chemical tools to over-express or suppress one or more genes or elements of these pathways can improve the efficiency and/or outcome of the methods of the invention. For example, it can be useful to over-express certain homologous recombination pathway genes or suppression of non-homologous pathway genes, depending upon the desired modification.

For example, gene function can be modified using antisense modulation using at least one antisense compound, including antisense DNA, antisense RNA, a ribozyme, DNAzyme, a locked nucleic acid (LNA) and an aptamer. In some embodiments the molecules are chemically modified. In other embodiments the antisense molecule is antisense DNA or an antisense DNA analog.

RNA interference (RNAi) is another method known in the art to reduce gene function in plants, which is mediated by RNA-induced silencing complex (RISC), a sequence-specific, multicomponent nuclease that destroys messenger RNAs homologous to the silencing trigger. RISC is known to contain short RNAs (approximately 22 nucleotides) derived from the double-stranded RNA trigger. The short-nucleotide RNA sequences are homologous to the target gene that is being suppressed. Thus, the short-nucleotide sequences appear to serve as guide sequences to instruct a multicomponent nuclease, RISC, to destroy the specific mRNAs. The dsRNA used to initiate RNAi, may be isolated from native source or produced by known means, e.g., transcribed from DNA. Plasmids and vectors for generating RNAi molecules against target sequence are now readily available from commercial sources.

DNAzyme molecules, enzymatic oligonucleotides, and mutagenesis are other commonly known methods for reducing gene function. Any available mutagenesis procedure can be used, including but not limited to, site-directed point mutagenesis, random point mutagenesis, in vitro or in vivo homologous recombination (DNA shuffling), uracil-containing templates, oligonucleotide-directed mutagenesis, phosphorothioate-modified DNA mutagenesis, mutagenesis using gapped duplex DNA, point mismatch repair, repair-deficient host strains, restriction-selection and restriction-purification, deletion mutagenesis, total gene synthesis, double-strand break repair, zinc-finger nucleases (ZFN), transcription activator-like effector nucleases (TALEN), any other mutagenesis procedure known to a person skilled in the art.

A skilled artisan would also appreciate that clustered regularly interspaced short palindromic repeats (CRISPR)/CRISPR associated protein (Cas) system comprises genome engineering tools based on the bacterial CRISPR/Cas prokaryotic adaptive immune system. This RNA-based technology is very specific and allows targeted cleavage of genomic DNA guided by a customizable small noncoding RNA, resulting in gene modifications by both non-homologous end joining (NHEJ) and homology-directed repair (HDR) mechanisms (Belhaj K. et al., 2013. Plant Methods 2013, 9:39). In some embodiments, a CRISPR/Cas system comprises a CRISPR/Cas9 system.

Methods for transformation of plant cells required for gene editing are well known in the art, and the selection of the most appropriate transformation technique for a particular embodiment of the invention may be determined by the practitioner. Suitable methods may include electroporation of plant protoplasts, liposome-mediated transformation, polyethylene glycol (PEG) mediated transformation, transformation using viruses, micro-injection of plant cells, micro-projectile bombardment of plant cells, and Agrobacterium tumefaciens mediated transformation. Transformation means introducing a nucleotide sequence in a plant in a manner to cause stable or transient expression of the sequence.

In plant transformation techniques (e.g., vacuum-infiltration, floral spraying or floral dip procedures) are well known in the art and may be used to introduce expression cassettes of the invention (typically in an Agrobacterium vector) into meristematic or germline cells of a whole plant. Such methods provide a simple and reliable method of obtaining transformants at high efficiency while avoiding the use of tissue culture. (see, e.g., Bechtold et at. 1993 C. R. Acad. Sci. 316:1194-1199; Chung et at. 2000 Transgenic Res. 9:471-476; Clough et at. 1998 Plant J. 16:735-743; and Desfeux et at. 2000 Plant Physiol 123:895-904). In these embodiments, seed produced by the plant comprise the expression cassettes encoding the genome editing proteins of the invention. The seed can be selected based on the ability to germinate under conditions that inhibit germination of the untransformed seed.

If transformation techniques require use of tissue culture, transformed cells may be regenerated into plants in accordance with techniques well known to those of skill in the art. The regenerated plants may then be grown, and crossed with the same or different plant varieties using traditional breeding techniques to produce seed, which are then selected under the appropriate conditions.

The expression cassette can be integrated into the genome of the plant cells, in which case subsequent generations will express the genome editing proteins of the invention. Alternatively, the expression cassette is not integrated into the genome of the plant's cell, in which case the genome editing proteins is transiently expressed in the transformed cells and is not expressed in subsequent generations.

A genome editing protein itself may be introduced into the plant cell. In these embodiments, the introduced genome editing protein is provided in sufficient quantity to modify the cell but does not persist after a contemplated period of time has passed or after one or more cell divisions. In such embodiments, no further steps are needed to remove or segregate away the genome editing protein and the modified cell. In these embodiments, the genome editing protein is prepared in vitro prior to introduction to a plant cell using well known recombinant expression systems (bacterial expression, in vitro translation, yeast cells, insect cells and the like). After expression, the protein is isolated, refolded if needed, purified and optionally treated to remove any purification tags, such as a His-tag. Once crude, partially purified, or more completely purified genome editing proteins are obtained, they may be introduced to a plant cell via electroporation, by bombardment with protein coated particles, by chemical transfection or by some other means of transport across a cell membrane.

The genome editing protein can also be expressed in Agrobacterium as a fusion protein, fused to an appropriate domain of a virulence protein that is translocated into plants (e.g., VirD2, VirE2, VirE2 and VirF). The Vir protein fused with the genome editing protein travels to the plant cell's nucleus, where the genome editing protein would produce the desired double stranded break in the genome of the cell. (see Vergunst et al. 2000 Science 290:979-82).

### Kits for Use in Diagnostic Applications

Kits for use in diagnostic, research, and prognostic applications are also provided by the invention. Such kits may include any or all of the following: assay reagents, buffers, nucleic acids for detecting the target sequences and other hybridization probes and/or primers. The kits may include instructional materials containing directions (i.e., protocols) for the practice of the methods of this invention. While the instructional materials typically comprise written or printed materials, they are not limited to such. Any medium capable of storing such instructions and communicating them to an end user is contemplated by this invention. Such media include, but are not limited to, electronic storage media (e.g., magnetic discs, tapes, cartridges, chips), optical media (e.g., CD ROM), cloud-based media, and the like. Such media may include addresses to internet sites that provide such instructional materials.

### Examples

Aspects of the present teachings can be further understood in light of the following examples, which should not be construed as limiting the scope of the present teachings in any way.

The practice of the present teachings employ, unless otherwise indicated, conventional methods of protein chemistry, biochemistry, recombinant DNA techniques and pharmacology, within the skill of the art. Such techniques are explained fully in the literature. See, e.g., T. Creighton, Proteins: Structures and Molecular Properties, 1993, W. Freeman and Co.; A. Lehninger, Biochemistry, Worth Publishers, Inc. (current addition); J. Sambrook et al., Molecular Cloning: A Laboratory Manual, 2nd Edition, 1989; Methods In Enzymology, S. Colowick and N. Kaplan, eds., Academic Press, Inc.; Remington's Pharmaceutical Sciences, 18th Edition, 1990, Mack Publishing Company, Easton, Pa.; Carey and Sundberg, Advanced Organic Chemistry, Vols. A and B, 3rd Edition, 1992, Plenum Press.

The practice of the present teachings also employ, unless otherwise indicated, conventional methods of statistical analysis, within the skill of the art. Such techniques are explained fully in the literature. See, e.g., J. Little and D. Rubin, Statistical Analysis with Missing Data, 2nd Edition 2002, John Wiley and Sons, Inc., NJ; M. Pepe, The Statistical Evaluation of Medical Tests for Classification and Prediction (Oxford Statistical Science Series) 2003, Oxford University Press, Oxford, UK; X. Zhoue et al., Statistical Methods in Diagnostic Medicine 2002, John Wiley and Sons, Inc., NJ; T. Hastie et. al, The Elements of Statistical Learning: Data Mining, Inference, and Prediction, Second Edition 2009, Springer, N.Y.; W. Cooley and P. Lohnes, Multivariate procedures for the behavioral science 1962, John Wiley and Sons, Inc. NY; E. Jackson, A User's Guide to Principal Components 2003, John Wiley and Sons, Inc., NY.

### Example 1—Discovery of Varin Markers

Markers associated with THCV, THCVA, CBDV, CBDVA, CBGV, or CBGVA production in Cannabis plants and their use in selecting Cannabis plants having modified THCV, THCVA, CBDV, CBDVA, CBGV, or CBGVA activity have been discovered, as described in PCT Patent Application No. PCT/US21/44908.

Additional markers associated with THCV, THCVA, CBDV, CBDVA, CBGV, or CBGVA production in Cannabis plants and their use in selecting Cannabis plants having modified THCV, THCVA, CBDV, CBDVA, CBGV, or CBGVA activity will be discovered, which will further determine causal genes responsible for modified varin activity.

### Example 2—Discovery of Varin Genes

Candidate genes with THCV, THCVA, CBDV, CBDVA, CBCV, CBCVA, CBGV, or CBGVA production in Cannabis plants and their use in selecting Cannabis plants having modified THCV, THCVA, CBDV, CBDVA, CBCV, CBCVA, CBGV, or CBGVA activity have been discovered, as described in PCT Patent Application No. PCT/US21/44908. Candidate genes may include, but are not limited to, RUN/FYVE domain protein (AT1G27850), 3-oxoacyl-[acyl-carrier-protein] reductase-chloroplastic (KR; AT1G24360 (SEQ ID NO:1 and SEQ ID NO:2), DYNAMIN-like 1C (DL1C; AT1G14830), DNA polymerase alpha-primase complex, polymerase-associated subunit B (POLA2; AT1G61580), Inositol polyphosphate multikinase alpha (IPK2a) and V-type proton ATPase subunit e1 (VHA-e1), BBE24, OMT1, Fatty Acyl-ACP Thioesterases B (FATB). Pentatricopeptide repeat-containing protein AT1G22960-mitochondrial, Origin of Replication Complex subunit 4 (ORC4), DNA repair and meiosis protein (MRE11), Actin-related protein 2/3 complex subunit 2A (ARPC2A), Membrane-bound transcription factor site-2 protease homolog (S2P), CTP synthase family protein, Aromatic aminotransferase ISS1 (ISS1), Pentatricopeptide repeat-containing protein AT1G33350 (PCMP-E57), G-type lectin S-receptor-like serine/threonine-protein kinase SD1-29 (SD129). Some of these candidate genes relative to their marker positions are described in Table 1.

TABLE 1
Five main varin SNP markers.

| | Candidate | | Position | Reference | Alternate | Beneficial |
|---|---|---|---|---|---|---|
| SNP marker | gene | Chrom . . . | (bp) | allele | allele | genotype |
| 142225_852 | BBE24 | 1 | 33,426,602 | G | C | G/C, C/C |
| 142078_3920202 | KR | 4 | 72,717,623 | C | A | C/A, A/A |
| 141488_308754 | FATB | 4 | 80,072,595 | A | G | A/G, G/G |
| 393_150329 | na | 6 | 56,278,544 | T | C | T/C, C/C |
| 140868_29987 | ARPC2A/ | 7 | 62,971,551 | C | T | C/T, T/T |
| | DIS2 | | | | | |
| 140868_8332 | ISS1 | 7 | 62,993,205 | G | A | G/A, A/A |

First column: SNP ID, second column: candidate gene, third column: chromosome of the Abacus reference genome (version CsaAba2) SNP marker is located on, fourth column: position on Abacus reference genome (version CsaAba2), fifth column: reference allele in the Abacus reference genome, sixth column: alternate allele, seventh column: beneficial genotype contributing to high levels of Total THCV, Total Varin, and/or Varin Ratio (except for the KR SNP marker all other SNP markers segregated for homozygous reference allele and heterozygous genotypes, it is expected that homozygous alternate allele genotypes have a similar beneficial effect on varin production).

### KR Gene

The KR gene pt/mtKR/FabG/FabG1 (β ketoacyl-acyl carrier protein (ACP) reductase, At1g24360) is also referred to as 3-oxoacyl-[acyl-carrier-protein] reductase (SEQ ID NO:1 and SEQ ID NO:2). KR functions together with enoyl-ACP reductase (pt/mtER) to catalyze two of the reactions that constitute the core four-reaction cycle of the short chain fatty acid biosynthesis (FAS) system, which iteratively elongates the acyl-chain by two carbon atoms per cycle (Guan, Xin, et al. “Dual-localized enzymatic components constitute the fatty acid synthase systems in mitochondria and plastids.” Plant Physiology 183.2 (2020): 517-529.). In Cannabis, plastid fatty acid biosynthesis forms the precursor for the acyl chain in THC and THCV (Welling, Matthew T., et al. “Complex patterns of cannabinoid alkyl side-chain inheritance in Cannabis.” Scientific reports 9.1 (2019): 1-13.), as well as CBD and CBDV, CBC and CBCV, CBG and CBGV. Allelic variation of KR likely produces a KR variant that results in a shorter propyl (3-carbon) side chain found in THCV, CBGV, CBCV, and CBDV, instead of a pentyl (5-carbon) group found in THC, CBC, CBG and CBD. Guan et al. (2020; Guan, Xin, et al. “Dual-localized enzymatic components constitute the fatty acid synthase systems in mitochondria and plastids.” Plant Physiology 183.2 (2020): 517-529.) describe a single copy of KR in Arabidopsis, the presence of a pre-sequence (transit peptide) determines whether it localizes to the chloroplast, where it is involved in fatty acid chain elongation, or to the mitochondria where it is involved in different processes. A T-DNA insertion in KR in Arabidopsis makes it embryonic lethal, further supporting the notion that it is essential for fatty acid biosynthesis. In Cannabis there appear to be at least 3 copies of KR with varying expression levels in stalked capitate trichomes between the low Total Varin (0.11%) variety ‘Purple Kush’ and the intermediate Total Varin variety ‘Finola’ (0.74%; Livingston et al. 2020, The Plant Journal 101:37-56). BLASTP results of the mapped ‘Abacus’ KR sequence on CBDRx reference genome result in: 3-oxoacyl-[acyl-carrier-protein] reductase 4 [Cannabis sativa] 100% protein sequence match for full 322 AA sequence. Gene expression analysis of Cannabis stalked capitate trichomes identifies 3 KR genes with homologs in Brassica napus: fabg1, fabg2, and fabg3. Two of these genes, fabg1 and fabg3 display significant expression differences between ‘Finola’ and ‘Purple Kush’ (Livingston et al. 2020, The Plant Journal 101:37-56). BLASTN of the transcriptome fragments of these three genes identified fabg1 in ‘Finola’ and ‘Purple Kush’ as the homolog of the mapped ‘Abacus’ KR gene.

TABLE 2
KR genomic DNA and predicted protein sequences.

| | | |
|---|---|---|
| SEQ ID | Cannabis KR gene | TATAAAGTAATTTTACAAGTATTATTATTATTATG |
| NO: 1 | sequence (genomic | CTCACCATAACAACAACACTCACAAAATAAATAAT |
| | DNA Abacus | TCCTTTGTCACAATTACTACTTACTTCCTAAGCAT |
| | reference genome) | TATACACATGCTAGAGTAATTCATCTCCATAAAAA |
| | | AAATATTCGTCACTATAAAATAAAATTTATTTGTC |
| | | ATTAAAATCCTTAATTCACAATATTCAAAATACTA |
| | | AAATGAAAAAAAAAATAAATGGAAGTAAAAATAGA |
| | | ATTAAATTCTTATTTTTTTTTTCCTTAAAAAAATG |
| | | GATATTACACCATTCTCATTGCTTTGTTATTGGTG |
| | | GTAGTTTAGAGGCTCCTCAATTATGCAGGGAGGGT |
| | | GGCCTACATTACCATCCCTCCATCAATGGTGAGAA |
| | | CCTACAAAACATGTTACAGAAAGTTCATTCAAGTA |
| | | TGAGAAGTTAAAATTGTTCAAAGAAAAAAAAAAAA |
| | | GAAAGTATGAGAAGTTAAAATCAATTAACTCTTCA |
| | | AAACCCAATTCATAGTAGGTGAAAGTGAAACCTGT |
| | | CCAGTTATGTAACTTGCAGCAGGACCAAGTGCCAA |
| | | AAATTCCACTAGCCCAGCAACTTCTTCTGGTTGAC |
| | | CATATCTTCCTGAAGAACAATAGAAATCACACTAA |
| | | TCTTCACATATTTTTCTCAACAATTCAGAGGAGTC |
| | | AGAGCCAAGTCGATATGAAGAAAGAACAGGATTGT |
| | | CTCAGCCTCACTCACCTAAGGGGATGCCCTCCAAG |
| | | TTTTTCTTCTCAATATCATCTCCTAGCTTGGCAGT |
| | | CATGTCAGATGCAATAAAGCCTGGAGCAATAGCAT |
| | | TAACCTGACAACAACAATTCAATATTAGTCATATT |
| | | GAAATATCTAATTATTTGAAAAAAAAAACTTTGGA |
| | | ATAACTCACAGTGATGTTTCTGCTGGAATACTCCT |
| | | TAGCAATAGCTTTTGTGAAGCCAATTACTCCTGCT |
| | | TTTGCAGCACTATAATTGGCTTGCCCGGCATTGCC |
| | | GACTAGACCGAAGACTGATGCTACATTGATTATCC |
| | | TTCCCTACAAAATTATTATTTGGGGACTAAGAAAA |
| | | TAAACAAAAAAACGAGACACAAAGTAAACTTCGGC |
| | | AAATCATTCTCAAAAGACTAAGCCTGTGAGAAAAT |
| | | AATGCCCAAGTGGTTATAAAAGTAGTCAATCCCAT |
| | | ACTCGATAAAGGATCCTAACAGTAAACCATTTTTA |
| | | GAGAGGATGTCCATGACAGCAACCCTCTTTGGGTT |
| | | TCTCCACAGGTCATCCTTTCCATGACATGGTCCCC |
| | | GAGAGGGTGCTCATTTTGATATTGAAATTATATGT |
| | | CGAAATATTCAATATAAAAATAGTTTTGACTGTAA |
| | | ATGGAGAGATAAAACGGTGTATATACCTTTTTCTT |
| | | CTTCATCATAATTTTGGCTGCAGCCTGCAAATAAA |
| | | GAATTAATTTTAGTTTCGAGAAAAAGTCAAGTTTG |
| | | AAGGACCTTAAGAAGTGAAAGCTTTAGATGCACAC |
| | | CTGTGTACATAGAAATACACCAGTAAGATTCAAAT |
| | | CAATGACATCCTGCCACTGGGATTTCTTCATTCTC |
| | | ATAATTAAGTTGTCCCTTGTGATTCCTGAAAACAC |
| | | CGAAAAAGCATCAGGAGATCGAATGTGGAATGAAA |
| | | ACAACATAAGAAAATGAAAGTTTCAAACAACCTGC |
| | | ATTATTTACTAATACATCAATAGTTCCCCAAGCAT |
| | | CAACTGCCTGCAGGTTTATGAGATGGAATATTACA |
| | | AACCATTCCACGGTTTAACATACAATGTGAAAAAA |
| | | CAATATGAAGAACATTTTCTCACTGTTTTGATCAT |
| | | TAATTCAACATCAGCTTCTTTTGAAACATCTCCTC |
| | | CAAAAGTAATAGCTTGGCCACCAGATGCTTCGATC |
| | | TGTAAGAACTCAGTCAAAATTATTTTTGATGGTGG |
| | | TTGAACTACATAAAGAAAATAAATACAAAAGTCTA |
| | | GAATCTACTAAATCAGGAAAGGAAAGGCATTTGAC |
| | | CAATTAAAAATTATGGAAGATAATTCTTGCACTGC |
| | | TGTAACTGAAACTATTAGTAATCAAATCATCACAG |
| | | AATTGAGGTTTGAATTGATGTCAAGTATTGTTATA |
| | | GAGAAGGAAAAAGAACTAATATACAAGTTAGAAAA |
| | | CATAAACATAAGAAAAAAATGACGCTCTTATATTC |
| | | ATTTTGAATCACATTATAAGTATAAATAATACACT |
| | | GCGTTTGGTTGGAGGTAATGAAATAGAATAAAAAG |
| | | GAAACAATATTTATTCTATTCTTTTGTTTGGTTGC |
| | | ATATTAAAGTATTGAAATATCATTCCAATGGAATT |
| | | GTCGTTCCACCATTTTGGTAGAGTGACTATTCTAT |
| | | TTTGAAATAAAAGGAAAGACTATTCCAATACACGA |
| | | TGAGAAAATATTTAATAATTTTTTTTATCAATTTT |
| | | TTTATGCATTTTAATTTTATTCCATTCTATTCCTA |
| | | TTCTTATTTCCATTCCCATTCTTATTCCTATGTTT |
| | | CCATTCTTCCCAACCAAACGCTACCTTAAATTCAA |
| | | AACATTGAAAGGGGCTACCCCTCTCCTCAAATTGT |
| | | ATGCAATGTGCTCTTAGATAATCTGAGTTATCTAC |
| | | ATTGTATATAATTTTCTGGTCCCAAATAATATTGC |
| | | AGGATATAAGGCAAGGAGCCTAAGTTTAAAAAAGG |
| | | GTAGCAAAAACATGAATTTAGGACTTCTACAATTG |
| | | CTAAAGTTTCCCACAATTGTAATTCCAATAGTTCT |
| | | GGGGCGCTAGTTTCAAAATATGCAGGGCCGGTTCT |
| | | GAAACAAAGTGGGTCACAAGAAAAAAATTTAGGGG |
| | | TAAGTTGGAAAATACCACTTTTATTAATCAATTAA |
| | | TCAAATTTACCTCTAATTTTATATTTAATTGAAAC |
| | | ATACCTCTTTTTATATGTATTGTACCTAAAATACC |
| | | CTGACATAAGAGAGTCGCATGGAGAGTATCTTGAA |
| | | GTGACAAGGGCAAAATGTGGTACAATGTTTAAAAA |
| | | AAAAAGAGGTAAAAATGATAGACTTTAAAAAAGAG |
| | | GGTAAAAATGAAAGAGGACAATATAAAAAGGGTAT |
| | | TGAGTGTAGTTTCCTCAAAAATTTATTTTTTGGCC |
| | | CTTATGTTAGAAAAATTATGGTATTTTTCAAATTC |
| | | AGTAAAAAAACTATATAATTTTAAAAGGGGAAAAA |
| | | AAAATTTGGCCCTAGGCCTAACCCGCATATCTATT |
| | | CTATATAAAGTGTGCCTATATAACCGAATTCTTGG |
| | | TTTATGAGAAATTCATGGGTGACTTTTTATTTATA |
| | | TTAAAAATAAAATGATTTATTATTAAAAATAAATT |
| | | TATATTAAGAAGCGAGAAGTATAAAACGATGACGT |
| | | GTCACTCTAAATTTATTCTAAATGTAACTTTCTAT |
| | | TCTCTCTTTAATTCTCTCATTTATATAATTTTTTT |
| | | AATCATAACTAAATTACTAATTATTTTTCAAAAAA |
| | | ATAAAACAAATAAAAATAATGGTATACTTCAAAAT |
| | | ATCACAAACTTAATACTAAATTTATTGTTTTCATA |
| | | AAACTAAATCATCCGTAGCTTATTTTTAATGATTT |
| | | TTTAATGTTTTATTTTAAAAAATATCCGTAGCTTT |
| | | TTTTAATGATTTTTTAATGTTTTATTTTAAAATAA |
| | | TAAAATTAAATTATCCATCACTAACAATTCATTTT |
| | | TTTTTTTGAAATTACTAACAATTCTTTTTTAAATA |
| | | TGCATCATTATATACTTTTTTTCTTAGCACATATC |
| | | CGTAGTTTTTTTTTTAATGATTTTTTATGTTTTAT |
| | | TTTTTTTCTTACATTTATATTATCTTTACAGATTA |
| | | ACAATAATTTTTTTTTTTTGTAAGGAGATTAACAA |
| | | TAAACTTTGATTCTCGATTCAAATATTATTTTTCA |
| | | ATTTTATTTTCATATATTCAAAATTCTCATTCTAT |
| | | AAATTTTTCACTAATATTTTGTAATCAATGAAACA |
| | | AAAATGCAAACTTTAATTCCCGTATGAAAAATCAA |
| | | ACTCTCAATCCCAATAATTAATAGAAACAACATCA |
| | | AGTTTCAAGTTTTCAAAATTTATAAAAATAAATAC |
| | | ATAAATAAATAAAAACATATCTCATAAACACAAAC |
| | | TCAAATATTAGAACCCCAGTAAAACACTAAAACTA |
| | | AAACATTAAAAACACACATATTAGAATAAAAATTA |
| | | AACCATAAATGAAAATTTATGCTACTATTTTTCAA |
| | | ATTTCGATATTTATTAGCAACAAATTTAATATTTT |
| | | AAGGTCACAAATTTTTTAATAGTAAATTCAAAATT |
| | | TTAATATAATTTTAATAAGTAATTATTTTTAAGTA |
| | | AAATATACAATTTATTTTTTATATATAATTTAAAA |
| | | TACTTTATACATCCGCCCGCCGGCGAACCGCGAAG |
| | | CACAGTATATATATATTTATATTTTATATTGAAAG |
| | | GGATAACACAAACATGAATAGAACTTGATGGAACT |
| | | CACCTCTTTGGAAACTTCCTCAGCCTCCTTTGATG |
| | | ATCTAGCATAATTAACAAGGACCTATAGTAATAGT |
| | | ATAGCACTATATCAGAAAAAGAAAAAACAAATTAC |
| | | ACACAACAAAGATCATCAATATTGCTAATAAAAAA |
| | | AAAAAACAAAGATCATCTCAATGAATTGGTAAAAG |
| | | GGAAAACCACAAACCTTACAACCAGCTTTTCCCAA |
| | | AGCCAATGCAGTAGCTTTTCCAATACCTCTAGAGG |
| | | CACCAGTCACAATGACCACCGGAGCCTCCACATTT |
| | | TGGCCTACTCCGGCGCTTGTTTCTTCAAGACTTGC |
| | | TCCCTGAGCTTTAACAACACCTGAAGAAGAAGAAG |
| | | AAGAAGAGGAACTACCATCATCGAAATCCCAAACC |
| | | CATTAAGAAAAACAAGTAATCGTACCACCGGAGGA |
| | | ACTCTTGGATTTGCACTGAAGGGAGATGGAGTGGG |
| | | GGAGTGATTGGCCACCGGAAATGGGGGACCACTGC |
| | | CGAAAATGTGGGGCTTTCCGGTCAAAGAATGAGCT |
| | | GGAAATTCTGGTTGATTTGGAAACTACAACGTTTG |
| | | ATCCAGCAATGGCAGCCATGGGATATATAGTTCCG |
| | | AAGATTGTTGTGGGCTTTGTACAAAGAAAAAGGCA |
| | | GAGTTTTTAGAAAACCCAGATAAGAAATTGAACTG |
| | | TTCCTTTTCGTGTTTACGTTGGTTTTTATATATTG |
| | | GGACTTTGGAAACTCCGAAGAGTGTTCGTACATCT |
| | | TTGACGACGGAAC |
| SEQ ID | Cannabis predicted | MAAIAGSNVVVSKSTRISSSFFDRKAPHFRQWSPI |
| NO: 2 | KR protein sequence | SGGQSLRHSISLQCKSKSSSGGVVKAQGASLEETS |
| | (CBDRx reference | AGVGQNVEAPVVIVTGASRGIGKATALALGKAGCK |
| | genome) | VLVNYARSSKEAEEVSKEIEASGGQAITFGGDVSK |
| | | EADVELMIKTAVDAWGTIDVLVNNAGITRDNLIMR |
| | | MKKSQWQDVIDLNLTGVFLCTQAAAKIMMKKKKGR |
| | | IINVASVFGLVGNAGQANYSAAKAGVIGFTKAIAK |
| | | EYSSRNITVNAIAPGFIASDMTAKLGDDIEKKNLE |
| | | GIPLGRYGQPEEVAGLVEFLALGPAASYITGQVLT |
| | | IDGGMVM |

First column: sequence ID,
second column: description,
third column: sequence.

### Example 3—Discovery of Varin Genetic Variants

KR was evaluated for variation in its coding sequence (CDS) in high and low varin producing accessions.

KR is located between positions 72,696,599-72,691,998 bp (start-stop codon) on chromosome 4 of the Abacus reference genome (version CsaAba2), its homolog on the CBDRx reference genome (version cs10) is LOC116714062, which is located between positions 6,585,812-6,590,794 bp on chromosome 4 of the CBDRx reference genome. Candidate genes were sequenced in a varin producing Cannabis accession which was part of the diversity panel used to map the varin markers (21VLP5-1-101; Table 3). Sequence data of this accession were compared with Abacus, CBDRx, Purple Kush, and Finola reference genome sequences; Abacus, CBDRx, and Purple Kush are considered non-varin producing accessions (0-0.1% Total Varin), whereas Finola is considered a varin producing accession (0.7% Total Varin; Table 3). In addition, KR was partially sequenced in several genetically different varin producing Cannabis accessions which were part of the diversity panel as well as the GAR2 F2 mapping population used to map the varin markers (Table 3).

RNA was extracted from flower tissue from 21VLP5-1-101 and Abacus collected 2-5 weeks after onset of flowering (Nucleospin RNA Plant and Fungi kit, Macherey-Nagel). Flower tissue was used in this experiment because varins as well as pentyl cannabinoids are produced in the stalked trichomes that can be found on flowers (Livingston, Samuel J., et al. “Cannabis glandular trichomes alter morphology and metabolite content during flower maturation.” The Plant Journal 101.1 (2020): 37-56.). After concentration adjustment and treatment with DNAse the RNA was used directly for RT-PCR (OneTaq® One-Step RT-PCR Kit, New England Biolabs). Sanger sequencing of CDS was performed based on RT-PCR product (NEB PCR® Cloning Kit; New England Biolabs). Genomic DNA (extracted from leaf tissue with a NucleoMag Plant DNA Kit, Macherey-Nagel) was used to sequence the beginning and end of each gene. Sanger sequencing based on cloned RT-PCR or PCR product was performed for areas with high levels of heterozygosity, Sanger sequencing based on RT-PCR or PCR product without cloning was performed for areas with low levels of heterozygosity. Primers for amplification and sequencing of fragments of KR can be found in Table 4.

TABLE 3
Accessions used for sequencing of varin gene KR.

| | Total | | KR marker | KR marker |
|---|---|---|---|---|
| | Varin | Varin | 142078_3920202 | 142078_3894722 |
| Accession name | (%) | Ratio | genotype | genotype |
| Abacus | 0.00 | 0.00 | 0/0 | 0/0 |
| Finola* | 0.74 | 0.32 | 1/1 | 0/1 |
| 21VLP5-1-101 | 4.23 | 3.71 | 1/1 | 1/1 |
| 21TX1-60 | 5.40 | 1.00 | 1/1 | 1/1 |
| 19GAR3-95-835 | 0.41 | 0.04 | 1/1 | 1/1 |
| 20VLP2-6-15 | 2.03 | 0.39 | 0/1 | 0/1 |
| 19GAR2-133-205 | 0.16 | 0.01 | 1/1 | 1/1 |

First column: accession name, *Public reference genome sequences were used for Finola, however, Illumina SNP array genotype data were generated; Total Varin % and Varin Ratio for this accession were obtained from Pavlovic, Radmila, et al. “Phytochemical and ecological analysis of two varieties of hemp (Cannabis sativa L.) grown in a mountain environment of Italian Alps.” Frontiers in Plant Science 10 (2019): 1265.
Second column: Total Varin (%; = Total THCV + Total CBDV + Total CBCV + Total CBGV) observed for flower tissue at maturity.
Third column: Varin Ratio (=Total Varin/(Total THC + Total CBD + Total CBC + Total CBG) observed for flower tissue at maturity.
Fourth and fifth columns: genotypes observed for significantly associated SNP markers located inside or near KRbased on mapping of Total Varin (%) and Varin Ratio (0/0 = homozygous reference genome, 0/1 = heterozygous, 1/1 = homozygous alternate allele, based on Abacus as the reference genome).

TABLE 4
Genomic DNA, coding, and protein sequences of KR and primers used for sequencing.

| SEQ ID NO | Description | Sequence |
|---|---|---|
| SEQ ID NO: 3 | KR1S_1AF | TCCGTCGTCAAAGATGTACG |
| SEQ ID NO: 4 | KR1S_1AR | GCACCAGTCACAATGACCAC |
| SEQ ID NO: 5 | KR1C_1AF | CACATTTTCGGCAGTGGTC |
| SEQ ID NO: 6 | KR1C_1AR | TTCCACTAGCCCAGCAACTT |
| SEQ ID NO: 7 | KR1E_AF | GCTCCAGGCTTTATTGCATC |
| SEQ ID NO: 8 | KR1E_BR | TGTCATTAAAATCCTTAATTCACAATA |
| SEQ ID NO: 9 | KR1K_BF | TCATTCTTTGACCGGAAAGC |
| SEQ ID NO: 10 | KR1K_BR | GGAAACTTCCTCAGCCTCCT |
| SEQ ID NO: 11 | KR1K2_BF | GACCTGTGGAGAAACCCAAA |
| SEQ ID NO: 12 | KR1K2_BR | TGCCAAAAATTCCACTAGCC |
| SEQ ID NO: 13 | 21VLP5-1-101 | ATGGCTGCCATTGCTGGATCAAACGTTGTCGTTTCCAAATCAACCGGAATTTCCAGCT |
| | KR CDS | CATTCTTTGACCGGAAAGCCCCACATTTTCGGCAGTGGTCCCCCATTYCCRGTGGCCA |
| | | ATCACTCCGTCACTCCATCTCCCTTCAATGCAAATCCAAGAGTTCSTCCGGTGGTGTT |
| | | GTKAAAGCTCAGGKTGMAASTCTTGAAGAAACAAGCGCCGGAGTAGGCCAAAATKTGG |
| | | AGGCTCCGGTGGTCATTGTGACTGGTGCCTCTAGAGGTATTRGAAAAGCTACTGCATT |
| | | GGCTTTGGGAAAAGCTGGTTGTAAGGTCCTTGTTAATTATGCTAGATCATCAAAGGAG |
| | | GCTGAGGAAGTTTCCAAAGARATTGAAGCATCTGGTGGCCAAGCTATTACTTTTGGAG |
| | | GAGATATTTCAAAAGAAGCTGATGTTGAATTAATGACCAAAATAGCAGTTGATGCTTG |
| | | GGGAACTGTTGATGTATTAGTAAATAATGCAGGAATCACTAGGGACAACTTAATGATG |
| | | AGAATGAAGAAATCCCAATGGCAGGATGTCATTGATTTGAATCTTACTGGTGTATTTY |
| | | TATGTACACAGGCTGCAGCSAAAATTATGATGAAGAAGAAAAAGGGAAGGATAATCAA |
| | | TGTAGCATCAATCTTCGGTTTAGTCGGCAATGCTGGGCAAGCCAATTRTAGTGCTGCA |
| | | AAAGCAGGAGTAATTGGCTTCACAAAAGCTGTTGCTAAGGAGTATTCCAGCAGAAACA |
| | | TCACTGTTAATGCTATTGCTCCAGGCTTTATTGCATCTGACATGACTGCCAAGCTAGG |
| | | AGATGATATTGAGAAGAAAAACTTGGAGGGCATCCCCTTAGGAAGATATGGTCAACCA |
| | | GAAGAAGTTGCTGGGCTAGTGGAATTTTTGGCACTTGATCCTGCTGCAAGTTACATAA |
| | | CTGGACAGGTTCTCACGATTGATGGAGGGATGGTAATGTAG |
| SEQ ID NO: 14 | Abacus KR | ATGGCTGCCATTGCTGGATCAAACGTTGTAGTTTCCAAATCAACCAGAATTTCCAGCT |
| | CDS* | CATTOTTTGACCGGAAAGCCCCACATTTTCGGCAGTGGTCCCCCATTTCCGGTGGCCA |
| | | ATCACTCCGCCACTCCATCTCCCTTCAGTGCAAATCCAAGAGTTCCTCCGGTGGTGTT |
| | | GTTAAAGCTCAGGGAGCAAGTCTTGAAGAAACAAGCGCCGGAGTAGGCCAAAATGTGG |
| | | AGGCTCCGGTGGTCATTGTGACTGGTGCCTCTAGAGGTATTGGAAAAGCTACTGCATT |
| | | GGCTTTGGGAAAAGCTGGTTGTAAGGTCCTTGTTAATTATGCTAGATCATCAAAGGAG |
| | | GCTGAGGAAGTTTCCAAAGAGATCGAAGCATCTGGTGGCCAAGCTATTACTTTTGGAG |
| | | GAGATGTTTCAAAAGAAGCTGATGTTGAATTAATGATCAAAACAGCAGTTGATGCTTG |
| | | GGGAACTGTTGATGTATTAGTAAATAATGCAGGAATCACAAGGGACAACTTAATTATG |
| | | AGAATGAAGAAATCCCAGTGGCAGGATGTCATTGATTTGAATCTTACTGGTGTATTTC |
| | | TATGTACACAGGCTGCAGCCAAAATTATGATGAAGAAGAAAAAGGGAAGGATAATCAA |
| | | TGTAGCATCAGTCTTCGGTCTAGTCGGCAATGCTGGGCAAGCCAATTACAGTGCTGCA |
| | | AAAGCAGGAGTAATTGGCTTCACAAAAGCTATTGCTAAGGAGTATTCCAGCAGAAACA |
| | | TCACTGTTAATGCTATTGCTCCAGGCTTTATTGCATCTGACATGACTGCCAAGCTAGG |
| | | AGATGATATTGAGAAGAAAAACTTGGAGGGCATCCCCTTAGGAAGATATGGTCAACCA |
| | | GAAGAAGTTGCTGGGCTAGTGGAATTTTTGGCACTTGGTCCTGCTGCAAGTTACATAA |
| | | CTGGACAGGTTCTCACCATTGATGGAGGGATGGTAATGTAG |
| SEQ ID NO: 15 | 21TX1-60 KR | ATGGCTGCCATTGCTGGATCAAACGTTGTAGTTTCCAAATCAACCGGAATTTCCAGCT |
| | CDS (last 66 | CATTCTTTGACCGGAAAGCCCCACATTTTCGGCAGTGGTCCCCCATTTCCGGTGGCCA |
| | bp at end of | ATCACTCCGTCACTCCATCTCCCTTCAATGCAAATCCAAGAGTTCCTCCGGTGGTGTT |
| | CDS | GTGAAAGCTCAGGGTGCAAGTCTTGAAGAAACAAGCGCCGGAGTAGGCCAAAATGTGG |
| | missing) | AGGCTCCGGTGGTCATTGTGACTGGTGCCTCTAGAGGTATTGGAAAAGCTACTGCATT |
| | | GGCTTTGGGAAAAGCTGGTTGTAAGGTACTTGTTAATTATGCTAPATCATCAAAGGAG |
| | | GCTGAGGAANNNNNNNNNNNNNNNNNNNNNNNNNNNNNNNNNNNNNNNNNNNNNNNNN |
| | | NNNNNNNNNNNNNNNNNNNNNNNNNNNNNNNNNNNNNNNNNNNNNNNNNNNNNNNNNN |
| | | NNNNNNNNNNNNNNNNNNNNNNNNNNNNNNNNNNNNNNNNNNNNNNNNNNNNNNNNNN |
| | | NNNNNNNNGAAATCCCAGTGGCAGGATGTCATTGATTTGAATCTTACTGGTGTATTTC |
| | | TATGTACACAGGCTGCAGCCAAAATTATGATGAAGAAGAAAAAGGGAAGGATAATCAA |
| | | TGTAGCATCAGTCTTCGGTCTAGTCGGCAATGCTGGGCAAGCCAATTACAGTGCTGCA |
| | | AAAGCAGGAGTAATTGGCTTCACAAAAGCTATTGCTAAGGAGTATTCCAGCAGAAACA |
| | | TCACTGTTAATGCTATTGCTCCAGGCTTTATTGCATCTGACATGACTGCCAAGCTAGG |
| | | AGATGATATTGAGAAGAAAAACTTGGAGGGCATCCCCTTAGGAAGATATGGTCAACCA |
| | | GAAGAAGTTGCTGGGCTAGTGGAATTTTTGGCA |
| SEQ ID NO: 16 | 21VLP5-1-101 | MAAIAGSNVVVSKSTGISSSFFDRKAPHFRQWSPIFSGQSLRHSISLQCKSKSSSGGV |
| | KR protein | VKAQVETLEETSAGVGQNLEAPVVIVIGASRGIRKATALALGKAGCKVLVNYARSSKE |
| | sequence | AEEVSKEIEASGGQAITFGGDISKEADVELMIKIAVDAWGTVDVLVNNAGITRDNIMM |
| | | RMKKSQWQDVIDLNLTGVFLCTQAAAKIMMKKKKGRIINVASIFGLVGNAGQANCSAA |
| | | KAGVIGFTKAVAKEYSSRNITVNAIAPGFIASDMTAKLGDDIEKKNLEGIPLGRYGQP |
| | | EEVAGLVEFLALDPAASYITGQVLTIDGGMVM* |
| SEQ ID NO: 17 | Abacus KR | MAAIAGSNVVVSKSTRISSSFFDRKAPHFRQWSPISGGQSLRHSISLQCKSKSSSGGV |
| | protein | VKAQGASLEETSAGVGQNVEAPVVIVIGASRGIGKATALALGKAGCKVLVNYARSSKE |
| | sequence | AEEVSKEIEASGGQAITFGGDVSKEADVELMIKTAVDAWGTIDVLVNNAGITRDNLIM |
| | | RMKKSQWQDVIDLNLIGVELCTQAAAKIMMKKKKGRIINVASVEGLVGNAGQANYSAA |
| | | KAGVIGETKAIAKEYSSRNITVNAIAPGFIASDMTAKLGDDIEKKNLEGIPLGRYGQP |
| | | EEVAGLVEFLALGPAASYITGQVLTIDGGMVM* |
| SEQ ID NO: | 21TX1-60 KR | MAAIAGSNVVVSKSTGISSSFFDRKAPHFRQWSPISGGQSLRHSISLQCKSKSSSGGV |
| 180 | protein | VKAQGASLEETSAGVGQNVEAPVVIVIGASRGIGKATALALGKAGCKVLVNYAKSSKE |
| | sequence | AEEXXXXXXXXXXXXXXXXXXXXXXXXXXXXXXXXXXXXXXXXXXXXXXXXXXXXXXX |
| | (last 22 AA at | XXXKSQWQDVIDLNLTGVFLCTQAAAKIMMKKKKGRIINVASVEGLVGNAGQANYSAA |
| | end of gene | KAGVIGFTKAIAKEYSSRNITVNAIAPGFIASDMTAKLGDDIEKKNLEGIPLGRYGQP |
| | missing) | EEVAGLVEFLA |
| SEQ ID NO: 19 | KR transit | ATCCCATGGCTGCCATTGCTGGATCAAACGTTGTAGTTTCCAAATCAACCAGAATTTC |
| | peptide SNP | CAGCTCATTCTTTGACCGGAAAGCCCCACATTTTCGGCAGTGG |
| | (A/G; position | |
| | 46 bp CDS) | |
| | at position 51 | |
| | bp | |
| SEQ ID NO: 20 | KR transit | CTCATTCTTTGACCGGAAAGCCCCACATTTTCGGCAGTGGTCCCCCATTTCCGGTGGC |
| | peptide SNP | CAATCACTCCGCCACTCCATCTCCCTTCAGTGCAAATCCAAGA |
| | (C/T; position | |
| | 107 bp CDS) | |
| | at position 51 | |
| | bp | |
| SEQ ID NO: 21 | KR transit | CATTCTTTGACCGGAAAGCCCCACATTTTCGGCAGTGGTCCCCCATTTCCGGTGGCCA |
| | peptide SNP | ATCACTCCGCCACTCCATCTCCCTTCAGTGCAAATCCAAGAGT |
| | (G/A; position | |
| | 109 bp CDS) | |
| | at position 51 | |
| | bp | |
| SEQ ID NO: 22 | KR SNP | TGGTAGTTCCTCTTCTTCTTCTTCTTCTTCAGGTGTTGTTAAAGCTCAGGGAGCAAGT |
| | (G/T; position | CTTGAAGAAACAAGCGCCGGAGTAGGCCAAAATGTGGAGGCTC |
| | 188 bp CDS) | |
| | at position 51 | |
| | bp | |
| SEQ ID NO: 23 | KR SNP | TAGTTCCTCTTCTTCTTCTTCTTCTTCAGGTGTTGTTAAAGCTCAGGGACAAGTCTTG |
| | (C/A; position | AAGAAACAAGCGCCGGAGTAGGCCAAAATGTGGAGGCTCCGG |
| | 191 bp CDS) | |
| | at position 51 | |
| | bp | |
| SEQ ID NO: 24 | KR SNP (G/C; | TTCCTCTTCTTCTTCTTCTTCTTCAGGTGTTGTTAAAGCTCAGGGAGCAAGTCTTGAA |
| | position 194 | GAAACAAGCGCCGGAGTAGGCCAAAATGTGGAGGCTCCGGTGG |
| | bp CDS) at | |
| | position 51 | |
| | bp | |
| SEQ ID NO: 25 | KR SNP | AAGCTCAGGGAGCAAGTCTTGAAGAAACAAGCGCCGGAGTAGGCCAAAATGTGGAGGC |
| | (G/T; position | TCCGGTGGTCATTGTGACTGGTGCCTCTAGAGGTATTGGAAAA |
| | 229 bp CDS) | |
| | at position 51 | |
| | bp | |
| SEQ ID NO: 26 | KR NADP | AAAATGTGGAGGCTCCGGTGGTCATTGTGACTGGTGCCTCTAGAGGTATTGGAAAAGC |
| | binding | TACTGCATTGGCTTTGGGAAAAGCTGGTTGTAAGGTTTGTGGT |
| | domain SNP | |
| | (G/A; position | |
| | 274 bp CDS) | |
| | at position 51 | |
| | bp | |
| SEQ ID NO: 27 | KR NADP | TTTCTGATATAGTGCTATACTATTACTATAGGTCCTTGTTAATTATGCTAGATCATCA |
| | binding | AAGGAGGCTGAGGAAGTTTCCAAAGAGGTGAGTTCCATCAAGT |
| | domain SNP | |
| | (G/A; position | |
| | 335 bp CDS) | |
| | at position 51 | |
| | bp | |
| SEQ ID NO: 28 | KR NADP | TCTTACAGATCGAAGCATCTGGTGGCCAAGCTATTACTTTTGGAGGAGATGTTTCAAA |
| | binding | AGAAGCTGATGTTGAATTAATGATCAAAACAGTGAGAAAATGT |
| | domain SNP | |
| | (G/A; position | |
| | 412 bp CDS) | |
| | at position 51 | |
| | bp | |
| SEQ ID NO: 29 | KR SNP | TATTACTTTTGGAGGAGATGTTTCAAAAGAAGCTGATGTTGAATTAATGATCAAAACA |
| | (T/C; position | GTGAGAAAATGTTCTTCATATTGTTTTTTCACATTGTATGTTA |
| | 443 bp CDS) | |
| | at position 51 | |
| | bp | |
| SEQ ID NO: 30 | KR SNP | TTTTGGAGGAGATGTTTCAAAAGAAGCTGATGTTGAATTAATGATCAAAACAGTGAGA |
| | (C/T; position | AAATGTTCTTCATATTGTTTTTTCACATTGTATGTTAAACCGT |
| | 449 bp CDS) | |
| | at position 51 | |
| | bp | |
| SEQ ID NO: 31 | KR SNP | TCTCCTGATGCTTTTTCGGTGTTTTCAGGAATCACAAGGGACAACTTAATTATGAGAA |
| | (T/G; position | TGAAGAAATCCCAGTGGCAGGATGTCATTGATTTGAATCTTAC |
| | 519 bp CDS) | |
| | at position 51 | |
| | bp | |
| SEQ ID NO: 32 | KR NADP | CAATGTAGCATCAGTCTTCGGTCTAGTCGGCAATGCCGGGCAAGCCAATTAYAGTGCT |
| | binding | GCAAAAGCAGGAGTAATTGGCTTCACAAAAGCTATTGCTAAGG |
| | domain SNP | |
| | (A/G; position | |
| | 686 bp CDS) | |
| | at position 51 | |
| | bp | |
| SEQ ID NO: 33 | KR SNP | AAGCCAATTAYAGTGCTGCAAAAGCAGGAGTAATTGGCTTCACAAAAGCTATTGCTAA |
| | (A/G; position | GGAGTATTCCAGCAGAAACATCACTGTGAGTTATTCCAAAGTT |
| | 727 bp CDS) | |
| | at position 51 | |
| | bp | |
| SEQ ID NO: 34 | KR SNP | ATATGGTCAACCAGAAGAAGTTGCTGGGCTAGTGGAATTTTTGGCACTTGGTCCTGCT |
| | (G/A; position | GCAAGTTACATAACTGGACAGGITTCACTTTCACCTACTATGA |
| | 908 bp CDS) | |
| | at position 51 | |
| | bp | |
| SEQ ID NO: 35 | KR transit | MAAIAGSNVVVSKSTGISSSFFDRKAPHFRQWSPISGGQSLRHSISLQCKSKSSSGGV |
| | peptide | VKAQGASLEETSAGVGQNVEAPVVIVIGASRGIGKATALALGKAGCKVLVNYARSSKE |
| | protein | AEEVSKEIEASGGQAITFGGDVSKEADVELMIKTAVDAWGTIDVLVNNAGITRDNLIM |
| | sequence (R | RMKKSQWQDVIDLNLTGVFLCTCAAAKIMMKKKKGRIINVASVEGLVGNAGQANYSAA |
| | to G; position | KAGVIGFTKAIAKEYSSRNITVNAIAPGFIASDMTAKLGDDIEKKNLEGIPLGRYGQP |
| | 16) | EEVAGLVEFLALGPAASYITGQVLTIDGGMVM |
| SEQ ID NO: 36 | KR transit | MAAIAGSNVVVSKSTRISSSFFDRKAPHFRQWSPIFGGQSLRHSISLQCKSKSSSGGV |
| | peptide | VKAQGASLEETSAGVGQNVEAPVVIVIGASRGIGKATALALGKAGCKVLVNYARSSKE |
| | protein | AEEVSKEIEASGGQAITFGGDVSKEADVELMIKTAVDAWGTIDVLVNNAGITRQNLIM |
| | sequence (S | RMKKSQWQDVIDLNLIGVFLCTQAAAKIMMKKKKGRIINVASVEGLVGNAGQANYSAA |
| | to F; position | KAGVIGFTKAIAKEYSSRNITVNAIAPGFIASDMTAKLGDDIEKKNLEGIPLGRYGQP |
| | 36) | EEVAGLVEFLALGPAASYITGQVLTIDGGMVM |
| SEQ ID NO: 37 | KR transit | MAAIAGSNVVVSKSTRISSSFFDRKAPHFRQWSPISSGQSLRHSISLQCKSKSSSGGV |
| | peptide | VKAQGASLEETSAGVGQNVEAPVVIVIGASRGIGKATALALGKAGCKVLVNYARSSKE |
| | protein | AEEVSKEIEASGGQAITFGGDVSKEADVELMIKTAVDAWGTIDVLVNNAGITRQNLIM |
| | sequence (G | RMKKSQWQDVIDLNLTGVFLCTQAAAKIMMKKKKGRIINVASVEGLVGNAGQANYSAA |
| | to S; position | KAGVIGFTKAIAKEYSSRNITVNAIAPGFIASDMTAKLGDDIEKKNLEGIPLGRYGQP |
| | 37) | EEVAGLVEFLALGPAASYITGQVLTIDGGMVM |
| SEQ ID NO: 38 | KR transit | MAAIAGSNVVVSKSTRISSSFFDRKAPHFRQWSPISGGQSLRHSISLQCKSKSSSGGV |
| | peptide | VKAQVASLEETSAGVGQNVEAPVVIVIGASRGIGKATALALGKAGCKVLVNYARSSKE |
| | protein | AEEVSKEIEASGGQAITFGGDVSKEADVELMIKTAVDAWGTIDVLVNNAGITRQNLIM |
| | sequence (G | RMKKSQWQDVIDLNLTGVFLCTCAAAKIMMKKKKGRIINVASVEGLVGNAGQANYSAA |
| | to V; position | KAGVIGFTKAIAKEYSSRNITVNAIAPGFIASDMTAKLGDDIEKKNLEGIPLGRYGQP |
| | 63) | EEVAGLVEFLALGPAASYITGQVLTIDGGMVM |
| SEQ ID NO: 39 | KR transit | MAAIAGSNVVVSKSTRISSSFFDRKAPHFRQWSPISGGQSLRHSISLQCKSKSSSGGV |
| | peptide | VKAQGESLEETSAGVGQNVEAPVVIVIGASRGIGKATALALGKAGCKVLVNYARSSKE |
| | protein | AEEVSKEIEASGGQAITFGGDVSKEADVELMIKTAVDAWGTVDVLVNNAGITRDNLIM |
| | sequence (A | RMKKSQWQDVIDLNLTGVFLCTQAAAKIMMKKKKGRIINVASVEGLVGNAGQANYSAA |
| | to E; position | KAGVIGFTKAIAKEYSSRNITVNAIAPGFIASDMTAKLGDDIEKKNLEGIPLGRYGQP |
| | 64) | EEVAGLVEFLALGPAASYITGQVLTIDGGMVM |
| SEQ ID NO: 40 | KR transit | MAAIAGSNVVVSKSTRISSSFFDRKAPHFRQWSPISGGQSLRHSISLQCKSKSSSGGV |
| | peptide | VKAQGATLEETSAGVGQNVEAPVVIVIGASRGIGKATALALGKAGCKVLVNYARSSKE |
| | protein | AEEVSKEIEASGGQAITFGGDVSKEADVELMIKTAVDAWGTIDVLVNNAGITRDNLIM |
| | sequence (S | RMKKSQWQDVIDLNLTGVFLCTQAAAKIMMKKKKGRIINVASVEGLVGNAGQANYSAA |
| | to T; position | KAGVIGFTKAIAKEYSSRNITVNAIAPGFIASDMTAKLGDDIEKKNLEGIPLGRYGQP |
| | 65) | EEVAGLVEFLALGPAASYITGQVLTIDGGMVM |
| SEQ ID NO: 41 | KR transit | MAAIAGSNVVVSKSTRISSSFFDRKAPHFRQWSPISGGQSLRHSISLQCKSKSSSGGV |
| | peptide | VKAQGASLEETSAGVGQNLEAPVVIVIGASRGIGKATALALGKAGCKVLVNYARSSKE |
| | protein | AEEVSKEIEASGGQAITFGGDVSKEADVELMIKTAVDAWGTIDVLVNNAGITRDNLIM |
| | sequence (V | RMKKSQWQDVIDLNLTGVELCTQAAAKIMMKKKKGRIINVASVEGLVGNAGQANYSAA |
| | to L; position | KAGVIGFTKAIAKEYSSRNITVNAIAPGFIASDMTAKLGDDIEKKNLEGIPLGRYGQP |
| | 77) | EEVAGLVEFLALGPAASYITGQVLTIDGGMVM |
| SEQ ID NO: 42 | KR transit | MAAIAGSNVVVSKSTRISSSFFDRKAPHFRQWSPISGGQSLRHSISLQCKSKSSSGGV |
| | peptide | VKAQGASLEETSAGVGQNVEAPVVIVIGASRGIRKATALALGKAGCKVLVNYARSSKE |
| | protein | ALEVSKEIEASGGQAITFGGDVSKEADVELMIKTAVDAWGTIDVLVNNAGITRDNLIM |
| | sequence (G | RMKKSQWQDVIDLNLTGVELCTQAAAKIMMKKKKGRIINVASVEGLVGNAGQANYSAA |
| | to R; position | KAGVIGETKAIAKEYSSRNITVNAIAPGFIASDMTAKLGDDIERKNLEGIPLGRYGQP |
| | 92) | EEVAGLVEFLALGPAASYITGQVLTIDGGMVM |
| SEQ ID NO: 43 | KR transit | MAAIAGSNVVVSKSTRISSSFFDRKAPHFRQWSPISGGQSLRHSISLQCKSKSSSGGV |
| | peptide | VKAQGASLEETSAGVGQNVEAPVVIVIGASRGIGKATALALGKAGCKVLVNYAKSSKE |
| | protein | AEEVSKEIEASGGQAITFGGDVSKEADVELMIKTAVDAWGTIDVLVNNAGITRDNLIM |
| | sequence (R | RMKKSQWQDVIDLNLTGVFLCTQAAAKIMMKKKKGRIINVASVFGLVGNAGQANYSAA |
| | to K; position | KAGVIGFTKAIAKEYSSRNITVNAIAPGFIASDMTAKLGDDIEKKNLEGIPLGRYGQP |
| | 112) | EEVAGIVEFLALGPAASYITGQVLTIDGGMVM |
| SEQ ID NO: 44 | KR transit | MAAIAGSNVVVSKSTRISSSFFDRKAPHFRQWSPISGGQSLRHSISLQCKSKSSSGGV |
| | peptide | VKAQGASLEETSAGVGQNVEAPVVIVIGASRGIGKATALALGKAGCKVLVNYARSSKE |
| | protein | AEEVSKEIEASGGQAITFGGDISKEADVELMIKTAVDAWGTIDVLVNNAGITRQNLIM |
| | sequence (V | RMKKSQWQDVIDLNLTGVFLCTQAAAKIMMKKKKGRIINVASVEGLVGNAGQANYSAA |
| | to I; position | KAGVIGFTKAIAKEYSSRNITVNAIAPGFIASDMTAKLGDDIEKKNLEGIPLGRYGQP |
| | 138) | EEVAGLVEFLALGPAASYITGQVLTIDGGMVM |
| SEQ ID NO: 45 | KR transit | MAAIAGSNVVVSKSTRISSSFFDRKAPHFRQWSPISGGQSLRHSISLQCKSKSSSGGV |
| | peptide | VKAQGASLEETSAGVGQNVEAPVVIVIGASRGIGKATALALGKAGCKVLVNYARSSKE |
| | protein | AEEVSKEIEASGGQAITFGGDVSKEADVELMIKTAVDAWGTIDVLVNNAGITRQNLIM |
| | sequence (I | RMKKSQWQDVIDLNLTGVFLCTCAAAKIMMKKKKGRIINVASVEGLVGNAGQANYSAA |
| | to T; position | KAGVIGETKAIAKEYSSRNITVNAIAPGFIASDMTAKLGDDIEKKNLEGIPLGRYGQP |
| | 148) | EEVAGLVEFLALGPAASYITGQVLTIDGGMVM |
| SEQ ID NO: 46 | KR transit | MAAIAGSNVVVSKSTRISSSFFDRKAPHFRQWSPISGGQSLRHSISLQCKSKSSSGGV |
| | peptide | VKAQGASLEETSAGVGQNVEAPVVIVIGASRGIGKATALALGKAGCKVLVNYARSSKE |
| | protein | AEEVSKEIEASGGQAITFGGDVSKEADVELMIKIAVDAWGTIDVLVNNAGITRDNLIM |
| | sequence (T | RMKKSQWQDVIDLNLTGVFLCTQAAAKIMMKKKKGRIINVASVEGLVGNAGQANYSAA |
| | to I; position | KAGVIGFTKAIAKEYSSRNITVNAIAPGFIASDMTAKLGDDIEKKNLEGIPLGRYGQP |
| | 150) | EEVAGLVEFLALGPAASYITGQVLTIDGGMVM |
| SEQ ID NO: 47 | KR transit | MAAIAGSNVVVSKSTRISSSFFDRKAPHFRQWSPISGGQSLRHSISLQCKSKSSSGGV |
| | peptide | VKAQGASLEETSAGVGQNVEAPVVIVTGASRGIGKATALALGKAGCKVLVNYARSSKE |
| | protein | AEEVSKEIEASGGQAITFGGDVSKEADVELMIKTAVDAWGTIDVLVNNAGITRDNIMM |
| | sequence (I | RMKKSQWQDVIDLNLTGVFLCTQAAAKIMMKKKKGRIINVASVEGLVGNAGQANYSAA |
| | to M; position | KAGVIGETKAIAKEYSSRNITVNAIAPGFIASDMTAKLGDDIEKKNLEGIPLGRYGQP |
| | 173) | EEVAGLVEFLALGPAASYITGQVLTIDGGMVM |
| SEQ ID NO: 48 | KR transit | MAAIAGSNVVVSKSTRISSSFFDRKAPHFRQWSPISGGQSLRHSISLQCKSKSSSGGV |
| | peptide | VKAQGASLEETSAGVGQNVEAPVVIVIGASRGIGKATALALGKAGCKVLVNYARSSKE |
| | protein | AEEVSKEIEASGGQAITFGGDVSKEADVELMIKTAVDAWGTIDVLVNNAGITRDNLIM |
| | sequence (Y | RMKKSQWQDVIDLNLTGVELCTQAAAKIMMKKKKGRIINVASVEGLVGNAGQANCSAA |
| | to C; position | KAGVIGFTKAIAKEYSSRNITVNAIAPGFIASDMTAKLGDDIEKKNLEGIPLGRYGQP |
| | 229) | EEVAGLVEFLALGFAASYITGQVLTIDGGMVM |
| SEQ ID NO: 49 | KR transit | MAAIAGSNVVVSKSTRISSSFFDRKAPHFRQWSPISGGQSLRHSISLQCKSKSSSGGV |
| | peptide | VKAQGASLEETSAGVGQNVEAPVVIVIGASRGIGKATALALGKAGCKVLVNYARSSKE |
| | protein | AEEVSKEIEASGGQAITFGGDVSKEADVELMIKTAVDAWGTIDVLVNNAGITRDNLIM |
| | sequence (I | RMKKSQWQDVIDLNLTGVELCTQAAAKIMMKKKKGRIINVASVEGLVGNAGQANYSAA |
| | to V; position | KAGVIGFTKAVAKEYSSRNITVNAIAPGFIASDMTAKLGDDIEKKNLEGIPLGRYGQP |
| | 243) | EEVAGLVEFLALGPAASYITGQVLTIDGGMVM |
| SEQ ID NO: 50 | KR transit | MAAIAGSNVVVSKSTRISSSFFDRKAPHFRQWSPISGGQSLRHSISLQCKSKSSSGGV |
| | peptide | VKAQGASLEETSAGVGQNVEAPVVIVIGASRGIGKATALALGKAGCKVLVNYARSSKE |
| | protein | AEEVSKEIEASGGQAITFGGDVSKEADVELMIKTAVDAWGTIDVLVNNAGITRDNLIM |
| | sequence (G | RMKKSQWQDVIDLNLTGVFLCTQAAAKIMMKKKKGRIINVASVFGLVGNAGQANYSAA |
| | to D; position | KAGVIGFTKAIAKEYSSRNITVNAIAPGFIASDMTAKLGDDIEKKNLEGIPLGRYGQP |
| | 303) | EEVAGLVEFLALDPAASYITGQVLTIDGGMVM |

First column: sequence ID;
second column: description of sequence (*Abacus CDS is not predicted, but actual CDS based on RT-PCR, start and end of gene are reference genome sequences,
third column: sequence where sequences of SNPs at position 51 bp are Abacus genomic DNA sequences.

Alignment of Sanger sequenced fragments was performed per accession. The resulting consensus sequences were subsequently aligned. Functional CDS were translated to protein sequences, which were subsequently aligned with Arabidopsis thaliana and Escherichia coli protein sequences. Functional domains were explored further in the protein sequence alignments for amino acid substitutions that would alter these domains and as a result the protein structure.

Alignment of functional KR CDS and protein sequences of varin and non-varin producing Cannabis accessions (SEQ ID NOs: 13-18; Table 4) revealed 17 amino acid substitutions in total, 16 were specific to one or more varin producing accessions (Table 5). Four amino acid substitutions were located in nucleotide binding (NADP) domains. The first three amino acid substitutions in nucleotide binding (NADP) domains were observed in a genetic background with high Total Varin greater than 4% and Varin Ratio greater than 3 (21VLP5-1-101; hybrids involving a sibling of 21VLP5-1-101 have Total Varin values up to 14.3%; Table 3): 1. G92R, a G (Glycine; conserved across A. thaliana and E. coli, observed in varin and non-varin producing Cannabis accessions) to R (Arginine; observed in 21VLP5-1-101) amino acid substitution at amino acid position 92 in a nucleotide binding (NADP) domain which is located between amino acid positions 84-108 in Cannabis based on homology with Arabidopsis (Uniprot annotation P33207 shows this domain to be located between 81-105 amino acids in A. thaliana). Accession 21VLP5-1-101 is heterozygous G/R. The causative SNP for this amino acid substitution is a G (varin and non-varin accessions) to A (G/A in 21VLP5-1-101) nucleotide substitution at position 274 bp from the start codon (SEQ ID NO:26; Table 4) 2. V1381, a V (Valine; conserved across A. thaliana and E. coli, observed in varin and non-varin producing Cannabis accessions) to I (Isoleucine; observed in 21VLP5-1-101) amino acid substitution at amino acid position 138, located in a nucleotide binding (NADP) domain which is located between amino acid positions 137-138 in Cannabis based on homology with E. coli (Uniprot annotation POAEK2 shows this domain to be located between amino acid positions 59-60 in E. coli). Accession 21VL5-1-101 is homozygous I/l at this position (Table 5). The causative SNP for this amino acid substitution is a G (varin and non-varin producing accessions) to A (A/A in 21VLP5-1-101) nucleotide substitution at position 412 from the start codon (SEQ ID NO:28; Table 4) 3. Y229C, a Y (Tyrosine; conserved across E. coli and A. thaliana, observed in varin and non-varin producing Cannabis accessions) to C (Cysteine; observed in 21VLP5-1-101) amino acid substitution at position 229, located at the active site (proton acceptor) in both A. thaliana (Uniprot annotation P33207 shows this site to be located at amino acid position 226 in Arabidopsis) and E. coli (Uniprot annotation POAEK2 shows this site to be located at amino acid position 151 in E. coli), which is part of an NADP binding domain between amino acid positions 229-233 in Cannabis based on homology with E. coli (Uniprot annotation POAEK2 shows this domain to be located between amino acid positions 151-155 in E. coli). Accession 21VL5-1-101 is heterozygous C/Y at this position (Table 5). The causative SNP for this amino acid substitution is an A (varin and non-varin producing accessions) to G (A/G in 21VLP5-1-101) nucleotide substitution at position 686 bp from the start codon (SEQ ID NO:32; Table 4).

The fourth nucleotide binding (NADP) domain amino acid substitution was observed for two different genetic backgrounds with Varin Ratio of lower or equal to one and Total Varin between 2.0-5.4% (21TX1-60 and 20VLP2-6-15; hybrids involving 21TX1-60 have Total Varin values up to 11.5%; Table 3): R112K, an R (Arginine; conserved in A. thaliana, observed in varin and non-varin producing Cannabis accessions) to K (Lysine; observed in 21TX1-60 and 20VLP2-6-15) substitution at position 112, located at a nucleotide binding (NADP) domain in E. coli (Uniprot annotation POAEK2 shows this site to be located at amino acid position 37 in E. coli). Accessions 21TX1-60 and 20VLP2-6-15 are heterozygous R/K at this position. The causative SNP for this amino acid substitution is a G (varin and non-varin producing accessions) to A (G/A in 21TX1-60 and 20VLP2-6-15) nucleotide substitution at position 335 bp from the start codon (SEQ ID NO:27; Table 4).

The four SNPs causing amino acid substitutions in four nucleotide binding (NADP) domains are expected to result in a defect in the affinity of the KR protein for the cofactor NADP (H), similar as has been observed for the E. coli FabG [Y151F] mutant (Price, Allen C., et al. “Cofactor-induced conformational rearrangements establish a catalytically competent active site and a proton relay conduit in FabG.” Structure 12.3 (2004): 417-428.). The Y (Tyrosine; E. coli wild type) to F (Phenylalanine; mutant E. coli) mutation at amino acid position 151 in E. coli, which is located in an NADP binding domain located between amino acid positions 151-155 in E. coli (Uniprot annotation POAEK2), resulted in mechanistically important conformation changes accompanying NADP (H) cofactor binding (Price, Allen C., et al. “Cofactor-induced conformational rearrangements establish a catalytically competent active site and a proton relay conduit in FabG.” Structure 12.3 (2004): 417-428.). The four NADP binding domain mutations observed in varin producing Cannabis accessions result in similar cofactor-induced conformational rearrangements leading to a reshaped catalytically competent active site as observed in E. coli FabG [Y151F] (Price, Allen C., et al. “Cofactor-induced conformational rearrangements establish a catalytically competent active site and a proton relay conduit in FabG.” Structure 12.3 (2004): 417-428.).

It is not expected that the NADP binding substitutions necessarily cause Cannabis plants to lose KR enzyme activity because the E. coli FabG [Y151F] mutant as well as other E. coli FabG NADP binding domain mutants retained high 3-ketoacyl-ACP reductase activity and were able to complete the initial cycle of fatty acid synthesis to produce butyryl-ACP although at lower enzymatic activity as a result of decreased affinity for NADPH binding (Hu, Zhe, et al. “Escherichia coli FabG 3-ketoacyl-ACP reductase proteins lacking the assigned catalytic triad residues are active enzymes.” Journal of Biological Chemistry 296 (2021).). Butyryl-ACP is converted to butanoic acid, the precursor molecule in varin production (Welling, Matthew T., et al. “Complex patterns of cannabinoid alkyl side-chain inheritance in Cannabis.” Scientific reports 9.1 (2019): 1-13.). Potentially the E. coli mutants described in Hu et al. (2021) are incapable of naturally producing fatty acids with chain lengths longer than the 4 carbon chain of butyryl-ACP, or the mutated KR enzyme possibly produces more butyryl-ACP as compared to longer chain lengths as has been shown for Pseudomonas sp. 61-3 FabG which has higher affinity for shorter carbon chain substrates in recombinant E. coli as compared to wild type E. coli (Nomura, Christopher T., et al. “Expression of 3-ketoacyl-acyl carrier protein reductase (fabG) genes enhances production of polyhydroxyalkanoate copolymer from glucose in recombinant Escherichia coli JM 109.” Applied and environmental microbiology 71.8 (2005): 4297-4306.).

It is expected that each additional defect NADP binding domain resulting from amino acid substitutions increases the extent of conformation changes of the KR protein and as a result the quantity of C4 relative to C6 fatty acids and therefore the quantity of varins as compared to pentyl cannabinoids. The KR protein in accessions with three defect NADP binding domains, such as 21VLP5-1-101, is expected to experience larger conformation changes as compared to the KR protein in accessions with one defect NADP binding domain, such as 21TX1-60 and 20VLP2-6-15. The Total Varin and Varin Ratio data support this as the accessions with amino acid substitutions affecting three NADP binding domains are characterized by Total Varin greater than 4% (of parents, up to 14.3% in hybrids) and high Varin Ratio greater than 3 (of parents; up to 5.2 in hybrids and up to 6.7 of selfs with a sibling of 21VLP5-1-101) as opposed to accessions with one NADP binding domain amino acid substitution which are characterized by Total Varin 2-5% (of parents, up to 11.5% in hybrids) and low Varin Ratio of less than or equal to 1 (of parents; up to 1.5 in hybrids; Table 3). In addition to these four amino acid substitutions in four nucleotide binding (NADP) domains, three amino acid substitutions were observed in the transit peptide domain which is located between amino acid positions 1-60 in Cannabis based on homology with Arabidopsis (Uniprot annotation P33207 shows this domain to be located between 1-57 amino acids in A. thaliana): 1. R16G, an R (Arginine; observed in all three non-varin producing Cannabis accessions) to G (Glycine; observed in all five varin producing Cannabis accessions) substitution at amino acid position 16. All five varin producing accessions are G/G (Table 5). The causative SNP for this amino acid substitution is an A to G nucleotide substitution at position 46 bp from the start codon (all five varin producing Cannabis accessions are G/G at this position; SEQ ID NO: 19; Table 4). 2. S36F, an S (Serine; observed in varin and non-varin producing Cannabis accessions) to F (Phenylalanine; observed in 21VLP5-1-101) substitution at amino acid position 36 where 21VLP5-1-101 is heterozygous F/S. The causative SNP for this amino acid substitution is a C to T nucleotide substitution at position 107 bp from the start codon (21VLP5-1-101 is C/T at this position; SEQ ID NO:20; Table 4). 3. G37S, a G (Glycine; conserved in A. thaliana, observed in varin and non-varin producing Cannabis accessions) to S (Serine; observed in 21VLP5-1-101) substitution at amino acid position 37 where 21VLP5-1-101 is heterozygous S/G (Table 5). The causative SNP for this amino acid substitution is a G to A nucleotide substitution at position 109 from the start codon (21VLP5-1-101 is G/A at this position; SEQ ID NO:21; Table 4).

The three SNPs causing amino acid substitutions in the transit peptide domain are expected to result in a defect in the affinity of the KR protein to mitochondria, similar as has been observed for Arabidopsis where C-terminus GFP-fused KR protein lacking the transit peptide domain accumulates to plastids (plastid signal is internal to the KR protein), whereas full-length KR protein targets both plastids and mitochondria (Guan, Xin, et al. “Dual-localized enzymatic components constitute the fatty acid synthase systems in mitochondria and plastids.” Plant Physiology 183.2 (2020): 517-529). DeepLoc 2.0 (DTU Health Tech) was used to predict subcellular localization of KR protein sequence of the varin producing accession 21VLP5-1-101 (SEQ ID NO:16) as compared to the non-varin producing accession Abacus (SEQ ID NO: 17). DeepLoc identified for both protein sequences plastid and mitochondrion localization, with a stronger signal of 0.99 for plastid for both sequences, however, the signal for mitochondrion was lower for 21VLP5-1-101 (0.17) as compared to Abacus (0.21), which is most likely the result of the transit peptide domain amino acid substitutions in 21VLP5-1-101, causing a reduced affinity for mitochondria. The increased affinity of the KR protein for chloroplasts results in decreased mitochondrial synthesis of the acyl precursor for the biosynthesis of lipoic acid and increased plastid synthesis of fatty acids which serve as precursor molecules for both cannabinoid and varin production. Arabidopsis plants containing KR without the transit peptide were not different from wild type when grown with ample CO2 (1% v/v atmosphere) to remedy the effect of reduced lipoylation status of the H-protein subunit of the glycine decarboxylase complex (Guan, Xin, et al. “Dual-localized enzymatic components constitute the fatty acid synthase systems in mitochondria and plastids.” Plant Physiology 183.2 (2020): 517-529.). Cannabis accessions containing amino acid substitutions in the transit peptide domain of KR experience the same level of mediation of this negative effect as Cannabis is usually grown with added CO2 to the greenhouse.

Because all varin producing Cannabis accessions share the R16G substitution (Table 5) it is expected that the underlying G nucleotide at position 46 bp from the start codon is required for varin production, however additional amino acid substitutions in this transit peptide domain are expected to be of benefit for varin production as well. Because accessions with only this nucleotide substitution or other nucleotide substitutions in the transit peptide domain produce lower Total Varin and low Varin Ratio, it is expected that these mutations are required to produce small quantities of varin but that one or more nucleotide substitutions in the four NADP binding domains are required for higher levels of Total Varin and Varin Ratio.

Of the 10 amino acid substitutions that are not in annotated domains, the G303D substitution is likely of importance for varin production because the D amino acid is shared among varin producing accessions, whereas the G amino acid is shared among non-varin producing accessions (Table 5). This amino acid substitution is near the amino acid mutation in E. coli which decreases thermostability of the KR protein (amino acid position 233 in E. coli; Uniprot annotation POAEK2). The causative SNP for this amino acid substitution is a G to A nucleotide substitution at position 908 bp from the start codon (SEQ ID NO:34; Table 4).

The remaining nine amino acid substitutions are located outside annotated domains in A. thaliana and E. coli (Table 5). However, because eight of these amino acid substitutions were specific to the high Total Varin and Varin Ratio accession 21VLP5-1-101 it is likely that one or more of the underlying nucleotide substitutions contribute to the observed high Total Varin and Varin Ratio values. These eight amino acid substitutions are the result of nine nucleotide substitutions at positions 188, 189, 191, 194, 229, 443, 449, 519, and 727 bp from the start codon (SEQ ID NOs: 22, 23, 24, 25, 29, 30, 31, and 33; Table 4).

TABLE 5
Varin-specific KR gene amino acid substitutions in accessions differing in their ability to produce varins.

| | | Producing varins | | | | | | Not producing varins | | |
|---|---|---|---|---|---|---|---|---|---|---|
| | | 21VLP5- | 21TX1- | 20VLP2- | | 19GAR3- | 19GAR2- | Purple | | |
| Domain | Position | 1-101 | 60 | 6-15 | Finola* | 95-835 | 133-205 | Kush* | CBDRX* | Abacus** |
| At_TP | 16 | G | G | NA | G | G | G | R | R | R |
| At_TP | 36 | F/S | S | S | S | S | S | S | S | S |
| At_TP | 37 | S/G | G | G | G | G | G | G | G | G |
| | 63 | V/G | G | G | G | G | G | G | G | G |
| | 64 | E/A | A | A | A | A | A | A | A | A |
| | 65 | T/S | S | S | S | S | S | S | S | S |
| | 77 | LAV | V | V | V | V | V | V | V | V |
| At_NB | 92 | R/G | G | G | G | G | NA | G | G | G |
| Ec_NB | 112 | R | K/R | K/R | R | NA | NA | R | R | R |
| Ec_NB | 138 | I | NA | NA | V | NA | NA | V | V | V |
| | 148 | T | NA | NA | I | NA | NA | I | I | I |
| | 150 | I | NA | NA | T | NA | NA | T | T | T |
| | 173 | M | NA | NA | I | I | I | I | I | I |
| At_AS; | 229 | C/Y | Y | Y | Y | Y | Y | Y | Y | Y |
| Ec_AS; | | | | | | | | | | |
| Ec_NB | | | | | | | | | | |
| | 243 | V | I | I | I | I | I | I | I | I |
| | 303 | D | NA | NA | NA | D | D | G | G | G |

First column: Uniprot annotated domains where amino acid substitutions reside (At = Arabidopsis thaliana, Ec = Escherichia coli; TP = transit peptide; NB = nucleotide binding, NADP; AS = active site, proton acceptor), second column: amino acid position in Cannabis, third through eight column: amino acid observed for varin producing Cannabis accessions (ordered from high to low Total Varin as well as Varin Ratio; NA = missing data,
*= public reference genome predicted CDS), ninth through eleventh column: amino acid observed for non-varin producing Cannabis accessions (*= public reference genome predicted CDS,
**= internal reference genome predicted CDS confirmed with experimental sequence data based on RNA).

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the invention as defined in the appended claims.

1. A transgenic Cannabis plant whose genome comprises a ketoacyl-acyl carrier protein reductase (KR) gene comprising one or more amino acid substitutions as compared to an endogenous KR gene and wherein the Cannabis plant comprises modified varin levels.

2. The transgenic Cannabis plant of claim 1 wherein the endogenous KR gene comprises a genomic nucleic acid sequence having at least 90% sequence identity to SEQ ID NO:14 or encodes an amino acid sequence having at least 90% sequence identity to SEQ ID NO:17.

3. The transgenic Cannabis plant of claim 2 wherein the KR gene comprises one or more of:
(a) a nucleotide polymorphism at position 46 of SEQ ID NO:14 that results in an Arginine to Glycine amino acid substitution at position 16 of SEQ ID NO:17;
(b) a nucleotide polymorphism at position 107 of SEQ ID NO:14 that results in a Serine to Phenylalanine amino acid substitution at position 36 of SEQ ID NO:17;
(c) a nucleotide polymorphism at position 109 of SEQ ID NO:14 that results in a Glycine to Serine amino acid substitution at position 37 of SEQ ID NO:17;
(d) a nucleotide polymorphism at position 188 of SEQ ID NO:14 that results in a Glycine to Valine amino acid substitution at position 63 of SEQ ID NO:17;
(e) a nucleotide polymorphism at position 191 of SEQ ID NO:14 that results in an Alanine to Glutamic Acid amino acid substitution at position 64 of SEQ ID NO:17;
(f) a nucleotide polymorphism at position 194 of SEQ ID NO:14 that results in a Serine to Threonine amino acid substitution at position 65 of SEQ ID NO:17;
(g) a nucleotide polymorphism at position 229 of SEQ ID NO:14 that results in a Valine to Leucine amino acid substitution at position 77 of SEQ ID NO:17;
(h) a nucleotide polymorphism at position 274 of SEQ ID NO:14 that results in a Glycine to Arginine amino acid substitution at position 92 of SEQ ID NO:17;
(i) a nucleotide polymorphism at position 335 of SEQ ID NO:14 that results in an Arginine to Lysine amino acid substitution at position 112 of SEQ ID NO:17;
(j) a nucleotide polymorphism at position 412 of SEQ ID NO:14 that results in a Valine to Isoleucine amino acid substitution at position 138 of SEQ ID NO:17;
(k) a nucleotide polymorphism at position 443 of SEQ ID NO:14 that results in an Isoleucine to Threonine amino acid substitution at position 148 of SEQ ID NO:17;
(l) a nucleotide polymorphism at position 449 of SEQ ID NO:14 that results in a Threonine to Isoleucine amino acid substitution at position 150 of SEQ ID NO:17;
(m) a nucleotide polymorphism at position 519 of SEQ ID NO:14 that results in an Isoleucine to Methionine amino acid substitution at position 173 of SEQ ID NO:17;
(n) a nucleotide polymorphism at position 686 of SEQ ID NO:14 that results in a Tyrosine to Cysteine amino acid substitution at position 229 of SEQ ID NO:17;
(o) a nucleotide polymorphism at position 727 of SEQ ID NO:14 that results in an Isoleucine to Valine amino acid substitution at position 243 of SEQ ID NO:17; or
(p) a nucleotide polymorphism at position 908 of SEQ ID NO:14 that results in a Glycine to Aspartic Acid amino acid substitution at position 303 of SEQ ID NO:17.

4. (canceled)

5. The transgenic Cannabis plant of claim 1 wherein the modified varin is elevated total varin.

6. The transgenic Cannabis plant of claim 5 wherein the elevated total varin is at least 2.0%.

7. The transgenic Cannabis plant of claim 6 wherein the elevated total varin is between 4.2% and 14.3%.

8. The transgenic Cannabis plant of claim 1 wherein the modified varin is an increased varin ratio.

9. The transgenic Cannabis plant of claim 8 wherein the increased varin ratio is at least 0.39.

10. The transgenic Cannabis plant of claim 9 wherein the increased varin ratio is between 0.39 and 6.7.

11. The transgenic Cannabis plant of claim 1 wherein the varin is one or more of tetrahydrocannabivarin (THCV), cannabigerivarin (CBGV), cannabichromevarin (CBCV), or cannabidivarin (CBDV).

12. A cell, seed, or flower isolated from the Cannabis plant of claim 1.

13. A Cannabis product made from the Cannabis plant of claim 1.

14. An isolated nucleic acid sequence encoding one or more amino acid substitutions of at least a portion of an endogenous Cannabis KR gene.

15-18. (canceled)

19. A method of making a Cannabis plant with modified varin levels, the method comprising replacing a nucleotide present within an endogenous KR gene with the isolated nucleic acid of claim 14.

20-28. (canceled)

29. The method of claim 19 wherein the replacing comprises gene editing, optionally wherein the gene editing comprises CRISPR technology.

30. (canceled)

31. A method for selecting one or more Cannabis plants having modified varin levels, the method comprising:
i) obtaining nucleic acids from a sample plant(s) or their germplasm;
(ii) detecting one or more markers that indicate a modified varin level phenotype,
(iii) indicating the modified varin level phenotype, and
(iv) selecting the one or more plants indicating the modified varin level phenotype.

32. (canceled)

33. The method of claim 31 wherein the one or more markers comprises a polymorphism at position 51 of any one or more of SEQ ID NO:19; SEQ ID NO:20; SEQ ID NO: 21; SEQ ID NO:22; SEQ ID NO:23; SEQ ID NO:24; SEQ ID NO:25; SEQ ID NO:26; SEQ ID NO: 27; SEQ ID NO:28; SEQ ID NO:29; SEQ ID NO:30; SEQ ID NO:31; SEQ ID NO:32; SEQ ID NO:33; or SEQ ID NO:34.

34-36. (canceled)

37. The method of claim 31 further comprising crossing the one or more plants comprising the indicated modified varin level phenotype to produce one or more F1 or additional progeny plants, wherein at least one of the F1 or additional progeny plants comprises the indicated modified varin level phenotype.

38. The method of claim 37 wherein the crossing comprises selfing, sibling crossing, or backcrossing, optionally wherein crossing further comprises marker-assisted selection.

39-46. (canceled)

47. The method of claim 31 wherein the varin is one or more of tetrahydrocannabivarin (THCV), cannabigerivarin (CBGV), cannabichromevarin (CBCV), or cannabidivarin (CBDV).

48. (canceled)


SEQUENCE LISTING (50 sequences)

SEQ ID NO: 1 - DNA, 5123 bp, Cannabis sativa
  source 1..5123: mol_type = genomic DNA
tataaagtaattttacaagtattattattattatgctcaccataacaacaacactcacaaaataaataattcctttgtcacaattactacttacttcctaagcattatacacatgctagagtaattcatctccataaaaaaaatattcgtcactataaaataaaatttatttgtcattaaaatccttaattcacaatattcaaaatactaaaatgaaaaaaaaaataaatggaagtaaaaatagaattaaattcttattttttttttccttaaaaaaatggatattacaccattctcattgctttgttattggtggtagtttagaggctcctcaattatgcagggagggtggcctacattaccatccctccatcaatggtgagaacctacaaaacatgttacagaaagttcattcaagtatgagaagttaaaattgttcaaagaaaaaaaaaaaagaaagtatgagaagttaaaatcaattaactcttcaaaacccaattcatagtaggtgaaagtgaaacctgtccagttatgtaacttgcagcaggaccaagtgccaaaaattccactagcccagcaacttcttctggttgaccatatcttcctgaagaacaatagaaatcacactaatcttcacatatttttctcaacaattcagaggagtcagagccaagtcgatatgaagaaagaacaggattgtctcagcctcactcacctaaggggatgccctccaagtttttcttctcaatatcatctcctagcttggcagtcatgtcagatgcaataaagcctggagcaatagcattaacctgacaacaacaattcaatattagtcatattgaaatatctaattatttgaaaaaaaaaactttggaataactcacagtgatgtttctgctggaatactccttagcaatagcttttgtgaagccaattactcctgcttttgcagcactataattggcttgcccggcattgccgactagaccgaagactgatgctacattgattatccttccctacaaaattattatttggggactaagaaaataaacaaaaaaacgagacacaaagtaaacttcggcaaatcattctcaaaagactaagcctgtgagaaaataatgcccaagtggttataaaagtagtcaatcccatactcgataaaggatcctaacagtaaaccatttttagagaggatgtccatgacagcaaccctctttgggtttctccacaggtcatcctttccatgacatggtccccgagagggtgctcattttgatattgaaattatatgtcgaaatattcaatataaaaatagttttgactgtaaatggagagataaaacggtgtatatacctttttcttcttcatcataattttggctgcagcctgcaaataaagaattaattttagtttcgagaaaaagtcaagtttgaaggaccttaagaagtgaaagctttagatgcacacctgtgtacatagaaatacaccagtaagattcaaatcaatgacatcctgccactgggatttcttcattctcataattaagttgtcccttgtgattcctgaaaacaccgaaaaagcatcaggagatcgaatgtggaatgaaaacaacataagaaaatgaaagtttcaaacaacctgcattatttactaatacatcaatagttccccaagcatcaactgcctgcaggtttatgagatggaatattacaaaccattccacggtttaacatacaatgtgaaaaaacaatatgaagaacattttctcactgttttgatcattaattcaacatcagcttcttttgaaacatctcctccaaaagtaatagcttggccaccagatgcttcgatctgtaagaactcagtcaaaattatttttgatggtggttgaactacataaagaaaataaatacaaaagtctagaatctactaaatcaggaaaggaaaggcatttgaccaattaaaaattatggaagataattcttgcactgctgtaactgaaactattagtaatcaaatcatcacagaattgaggtttgaattgatgtcaagtattgttatagagaaggaaaaagaactaatatacaagttagaaaacataaacataagaaaaaaatgacgctcttatattcattttgaatcacattataagtataaataatacactgcgtttggttggaggtaatgaaatagaataaaaaggaaacaatatttattctattcttttgtttggttgcatattaaagtattgaaatatcattccaatggaattgtcgttccaccattttggtagagtgactattctattttgaaataaaaggaaagactattccaatacacgatgagaaaatatttaataattttttttatcaatttttttatgcattttaattttattccattctattcctattcttatttccattcccattcttattcctatgtttccattcttcccaaccaaacgctaccttaaattcaaaacattgaaaggggctacccctctcctcaaattgtatgcaatgtgctcttagataatctgagttatctacattgtatataattttctggtcccaaataatattgcaggatataaggcaaggagcctaagtttaaaaaagggtagcaaaaacatgaatttaggacttctacaattgctaaagtttcccacaattgtaattccaatagttctggggcgctagtttcaaaatatgcagggccggttctgaaacaaagtgggtcacaagaaaaaaatttaggggtaagttggaaaataccacttttattaatcaattaatcaaatttacctctaattttatatttaattgaaacatacctctttttatatgtattgtacctaaaataccctgacataagagagtcgcatggagagtatcttgaagtgacaagggcaaaatgtggtacaatgtttaaaaaaaaaagaggtaaaaatgatagactttaaaaaagagggtaaaaatgaaagaggacaatataaaaagggtattgagtgtagtttcctcaaaaatttattttttggcccttatgttagaaaaattatggtatttttcaaattcagtaaaaaaactatataattttaaaaggggaaaaaaaaatttggccctaggcctaacccgcatatctattctatataaagtgtgcctatataaccgaattcttggtttatgagaaattcatgggtgactttttatttatattaaaaataaaatgatttattattaaaaataaatttatattaagaagcgagaagtataaaacgatgacgtgtcactctaaatttattctaaatgtaactttctattctctctttaattctctcatttatataatttttttaatcataactaaattactaattatttttcaaaaaaataaaacaaataaaaataatggtatacttcaaaatatcacaaacttaatactaaatttattgttttcataaaactaaatcatccgtagcttatttttaatgattttttaatgttttattttaaaaaatatccgtagctttttttaatgattttttaatgttttattttaaaataataaaattaaattatccatcactaacaattcatttttttttttgaaattactaacaattcttttttaaatatgcatcattatatactttttttcttagcacatatccgtagttttttttttaatgattttttatgttttattttttttcttacatttatattatctttacagattaacaataattttttttttttgtaaggagattaacaataaactttgattctcgattcaaatattatttttcaattttattttcatatattcaaaattctcattctataaatttttcactaatattttgtaatcaatgaaacaaaaatgcaaactttaattcccgtatgaaaaatcaaactctcaatcccaataattaatagaaacaacatcaagtttcaagttttcaaaatttataaaaataaatacataaataaataaaaacatatctcataaacacaaactcaaatattagaaccccagtaaaacactaaaactaaaacattaaaaacacacatattagaataaaaattaaaccataaatgaaaatttatgctactatttttcaaatttcgatatttattagcaacaaatttaatattttaaggtcacaaattttttaatagtaaattcaaaattttaatataattttaataagtaattatttttaagtaaaatatacaatttattttttatatataatttaaaatactttatacatccgcccgccggcgaaccgcgaagcacagtatatatatatttatattttatattgaaagggataacacaaacatgaatagaacttgatggaactcacctctttggaaacttcctcagcctcctttgatgatctagcataattaacaaggacctatagtaatagtatagcactatatcagaaaaagaaaaaacaaattacacacaacaaagatcatcaatattgctaataaaaaaaaaaaacaaagatcatctcaatgaattggtaaaagggaaaaccacaaaccttacaaccagcttttcccaaagccaatgcagtagcttttccaatacctctagaggcaccagtcacaatgaccaccggagcctccacattttggcctactccggcgcttgtttcttcaagacttgctccctgagctttaacaacacctgaagaagaagaagaagaagaggaactaccatcatcgaaatcccaaacccattaagaaaaacaagtaatcgtaccaccggaggaactcttggatttgcactgaagggagatggagtggcggagtgattggccaccggaaatgggggaccactgccgaaaatgtggggctttccggtcaaagaatgagctggaaattctggttgatttggaaactacaacgtttgatccagcaatggcagccatgggatatatagttccgaagattgttgtgggctttgtacaaagaaaaaggcagagtttttagaaaacccagataagaaattgaactgttccttttcgtgtttacgttggtttttatatattgggactttggaaactccgaagagtgttcgtacatctttgacgacggaac

SEQ ID NO: 2 - protein, 322 aa, Cannabis sativa
MAAIAGSNVVVSKSTRISSSFFDRKAPHFRQWSPISGGQSLRHSISLQCKSKSSSGGVVKAQGASLEETSAGVGQNVEAPVVIVTGASRGIGKATALALGKAGCKVLVNYARSSKEAEEVSKEIEASGGQAITFGGDVSKEADVELMIKTAVDAWGTIDVLVNNAGITRDNLIMRMKKSQWQDVIDLNLTGVFLCTQAAAKIMMKKKKGRIINVASVFGLVGNAGQANYSAAKAGVIGFTKAIAKEYSSRNITVNAIAPGFIASDMTAKLGDDIEKKNLEGIPLGRYGQPEEVAGLVEFLALGPAASYITGQVLTIDGGMVM

SEQ ID NO: 3 - DNA, 20 bp, Cannabis sativa
  source 1..20: mol_type = genomic DNA
tccgtcgtcaaagatgtacg

SEQ ID NO: 4 - DNA, 20 bp, Cannabis sativa
  source 1..20: mol_type = genomic DNA
gcaccagtcacaatgaccac

SEQ ID NO: 5 - DNA, 19 bp, Cannabis sativa
  source 1..19: mol_type = genomic DNA
cacattttcggcagtggtc

SEQ ID NO: 6 - DNA, 20 bp, Cannabis sativa
  source 1..20: mol_type = genomic DNA
ttccactagcccagcaactt

SEQ ID NO: 7 - DNA, 20 bp, Cannabis sativa
  source 1..20: mol_type = genomic DNA
gctccaggctttattgcatc

SEQ ID NO: 8 - DNA, 27 bp, Cannabis sativa
  source 1..27: mol_type = genomic DNA
tgtcattaaaatccttaattcacaata

SEQ ID NO: 9 - DNA, 20 bp, Cannabis sativa
  source 1..20: mol_type = genomic DNA
tcattctttgaccggaaagc

SEQ ID NO: 10 - DNA, 20 bp, Cannabis sativa
  source 1..20: mol_type = genomic DNA
ggaaacttcctcagcctcct

SEQ ID NO: 11 - DNA, 20 bp, Cannabis sativa
  source 1..20: mol_type = genomic DNA
gacctgtggagaaacccaaa

SEQ ID NO: 12 - DNA, 20 bp, Cannabis sativa
  source 1..20: mol_type = genomic DNA
tgccaaaaattccactagcc

SEQ ID NO: 13 - RNA, 969 nt, Cannabis sativa
  source 1..969: mol_type = transcribed RNA
atggctgccattgctggatcaaacgttgtcgtttccaaatcaaccggaatttccagctcattctttgaccggaaagccccacattttcggcagtggtcccccattyccrgtggccaatcactccgtcactccatctcccttcaatgcaaatccaagagttcstccggtggtgttgtkaaagctcaggktgmaastcttgaagaaacaagcgccggagtaggccaaaatktggaggctccggtggtcattgtgactggtgcctctagaggtattrgaaaagctactgcattggctttgggaaaagctggttgtaaggtccttgttaattatgctagatcatcaaaggaggctgaggaagtttccaaagarattgaagcatctggtggccaagctattacttttggaggagatatttcaaaagaagctgatgttgaattaatgaccaaaatagcagttgatgcttggggaactgttgatgtattagtaaataatgcaggaatcactagggacaacttaatgatgagaatgaagaaatcccaatggcaggatgtcattgatttgaatcttactggtgtatttytatgtacacaggctgcagcsaaaattatgatgaagaagaaaaagggaaggataatcaatgtagcatcaatcttcggtttagtcggcaatgctgggcaagccaattrtagtgctgcaaaagcaggagtaattggcttcacaaaagctgttgctaaggagtattccagcagaaacatcactgttaatgctattgctccaggctttattgcatctgacatgactgccaagctaggagatgatattgagaagaaaaacttggagggcatccccttaggaagatatggtcaaccagaagaagttgctgggctagtggaatttttggcacttgatcctgctgcaagttacataactggacaggttctcacgattgatggagggatggtaatgtag

SEQ ID NO: 14 - RNA, 969 nt, Cannabis sativa
  source 1..969: mol_type = transcribed RNA
atggctgccattgctggatcaaacgttgtagtttccaaatcaaccagaatttccagctcattctttgaccggaaagccccacattttcggcagtggtcccccatttccggtggccaatcactccgccactccatctcccttcagtgcaaatccaagagttcctccggtggtgttgttaaagctcagggagcaagtcttgaagaaacaagcgccggagtaggccaaaatgtggaggctccggtggtcattgtgactggtgcctctagaggtattggaaaagctactgcattggctttgggaaaagctggttgtaaggtccttgttaattatgctagatcatcaaaggaggctgaggaagtttccaaagagatcgaagcatctggtggccaagctattacttttggaggagatgtttcaaaagaagctgatgttgaattaatgatcaaaacagcagttgatgcttggggaactgttgatgtattagtaaataatgcaggaatcacaagggacaacttaattatgagaatgaagaaatcccagtggcaggatgtcattgatttgaatcttactggtgtatttctatgtacacaggctgcagccaaaattatgatgaagaagaaaaagggaaggataatcaatgtagcatcagtcttcggtctagtcggcaatgctgggcaagccaattacagtgctgcaaaagcaggagtaattggcttcacaaaagctattgctaaggagtattccagcagaaacatcactgttaatgctattgctccaggctttattgcatctgacatgactgccaagctaggagatgatattgagaagaaaaacttggagggcatccccttaggaagatatggtcaaccagaagaagttgctgggctagtggaatttttggcacttggtcctgctgcaagttacataactggacaggttctcaccattgatggagggatggtaatgtag

SEQ ID NO: 15 - RNA, 903 nt, Cannabis sativa
  source 1..903: mol_type = transcribed RNA
atggctgccattgctggatcaaacgttgtagtttccaaatcaaccggaatttccagctcattctttgaccggaaagccccacattttcggcagtggtcccccatttccggtggccaatcactccgtcactccatctcccttcaatgcaaatccaagagttcctccggtggtgttgtgaaagctcagggtgcaagtcttgaagaaacaagcgccggagtaggccaaaatgtggaggctccggtggtcattgtgactggtgcctctagaggtattggaaaagctactgcattggctttgggaaaagctggttgtaaggtacttgttaattatgctaratcatcaaaggaggctgaggaannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngaaatcccagtggcaggatgtcattgatttgaatcttactggtgtatttctatgtacacaggctgcagccaaaattatgatgaagaagaaaaagggaaggataatcaatgtagcatcagtcttcggtctagtcggcaatgctgggcaagccaattacagtgctgcaaaagcaggagtaattggcttcacaaaagctattgctaaggagtattccagcagaaacatcactgttaatgctattgctccaggctttattgcatctgacatgactgccaagctaggagatgatattgagaagaaaaacttggagggcatccccttaggaagatatggtcaaccagaagaagttgctgggctagtggaatttttggca

SEQ ID NO: 16 - protein, 322 aa, Cannabis sativa
MAAIAGSNVVVSKSTGISSSFFDRKAPHFRQWSPIFSGQSLRHSISLQCKSKSSSGGVVKAQVETLEETSAGVGQNLEAPVVIVTGASRGIRKATALALGKAGCKVLVNYARSSKEAEEVSKEIEASGGQAITFGGDISKEADVELMTKIAVDAWGTVDVLVNNAGITRDNLMMRMKKSQWQDVIDLNLTGVFLCTQAAAKIMMKKKKGRIINVASIFGLVGNAGQANCSAAKAGVIGFTKAVAKEYSSRNITVNAIAPGFIASDMTAKLGDDIEKKNLEGIPLGRYGQPEEVAGLVEFLALDPAASYITGQVLTIDGGMVM

SEQ ID NO: 17 - protein, 322 aa, Cannabis sativa; identical to SEQ ID NO: 2

SEQ ID NO: 18 - protein, 301 aa, Cannabis sativa
MAAIAGSNVVVSKSTGISSSFFDRKAPHFRQWSPISGGQSLRHSISLQCKSKSSSGGVVKAQGASLEETSAGVGQNVEAPVVIVTGASRGIGKATALALGKAGCKVLVNYAKSSKEAEEXXXXXXXXXXXXXXXXXXXXXXXXXXXXXXXXXXXXXXXXXXXXXXXXXXXXXXXXXXKSQWQDVIDLNLTGVFLCTQAAAKIMMKKKKGRIINVASVFGLVGNAGQANYSAAKAGVIGFTKAIAKEYSSRNITVNAIAPGFIASDMTAKLGDDIEKKNLEGIPLGRYGQPEEVAGLVEFLA

SEQ ID NO: 19 - DNA, 101 bp, Cannabis sativa
  source 1..101: mol_type = genomic DNA
atcccatggctgccattgctggatcaaacgttgtagtttccaaatcaaccagaatttccagctcattctttgaccggaaagccccacattttcggcagtgg

SEQ ID NO: 20 - DNA, 101 bp, Cannabis sativa
  source 1..101: mol_type = genomic DNA
ctcattctttgaccggaaagccccacattttcggcagtggtcccccatttccggtggccaatcactccgccactccatctcccttcagtgcaaatccaaga

SEQ ID NO: 21 - DNA, 101 bp, Cannabis sativa
  source 1..101: mol_type = genomic DNA
cattctttgaccggaaagccccacattttcggcagtggtcccccatttccggtggccaatcactccgccactccatctcccttcagtgcaaatccaagagt

SEQ ID NO: 22 - DNA, 101 bp, Cannabis sativa
  source 1..101: mol_type = genomic DNA
tggtagttcctcttcttcttcttcttcttcaggtgttgttaaagctcagggagcaagtcttgaagaaacaagcgccggagtaggccaaaatgtggaggctc

SEQ ID NO: 23 - DNA, 101 bp, Cannabis sativa
  source 1..101: mol_type = genomic DNA
tagttcctcttcttcttcttcttcttcaggtgttgttaaagctcagggagcaagtcttgaagaaacaagcgccggagtaggccaaaatgtggaggctccgg

SEQ ID NO: 24 - DNA, 101 bp, Cannabis sativa
  source 1..101: mol_type = genomic DNA
ttcctcttcttcttcttcttcttcaggtgttgttaaagctcagggagcaagtcttgaagaaacaagcgccggagtaggccaaaatgtggaggctccggtgg

SEQ ID NO: 25 - DNA, 101 bp, Cannabis sativa
  source 1..101: mol_type = genomic DNA
aagctcagggagcaagtcttgaagaaacaagcgccggagtaggccaaaatgtggaggctccggtggtcattgtgactggtgcctctagaggtattggaaaa

SEQ ID NO: 26 - DNA, 101 bp, Cannabis sativa
  source 1..101: mol_type = genomic DNA
aaaatgtggaggctccggtggtcattgtgactggtgcctctagaggtattggaaaagctactgcattggctttgggaaaagctggttgtaaggtttgtggt

SEQ ID NO: 27 - DNA, 101 bp, Cannabis sativa
  source 1..101: mol_type = genomic DNA
tttctgatatagtgctatactattactataggtccttgttaattatgctagatcatcaaaggaggctgaggaagtttccaaagaggtgagttccatcaagt

SEQ ID NO: 28 - DNA, 101 bp, Cannabis sativa
  source 1..101: mol_type = genomic DNA
tcttacagatcgaagcatctggtggccaagctattacttttggaggagatgtttcaaaagaagctgatgttgaattaatgatcaaaacagtgagaaaatgt

SEQ ID NO: 29 - DNA, 101 bp, Cannabis sativa
  source 1..101: mol_type = genomic DNA
tattacttttggaggagatgtttcaaaagaagctgatgttgaattaatgatcaaaacagtgagaaaatgttcttcatattgttttttcacattgtatgtta

SEQ ID NO: 30 - DNA, 101 bp, Cannabis sativa
  source 1..101: mol_type = genomic DNA
ttttggaggagatgtttcaaaagaagctgatgttgaattaatgatcaaaacagtgagaaaatgttcttcatattgttttttcacattgtatgttaaaccgt

SEQ ID NO: 31 - DNA, 101 bp, Cannabis sativa
  source 1..101: mol_type = genomic DNA
tctcctgatgctttttcggtgttttcaggaatcacaagggacaacttaattatgagaatgaagaaatcccagtggcaggatgtcattgatttgaatcttac

SEQ ID NO: 32 - DNA, 101 bp, Cannabis sativa
  source 1..101: mol_type = genomic DNA
caatgtagcatcagtcttcggtctagtcggcaatgccgggcaagccaattayagtgctgcaaaagcaggagtaattggcttcacaaaagctattgctaagg

SEQ ID NO: 33 - DNA, 101 bp, Cannabis sativa
  source 1..101: mol_type = genomic DNA
aagccaattayagtgctgcaaaagcaggagtaattggcttcacaaaagctattgctaaggagtattccagcagaaacatcactgtgagttattccaaagtt

SEQ ID NO: 34 - DNA, 101 bp, Cannabis sativa
  source 1..101: mol_type = genomic DNA
atatggtcaaccagaagaagttgctgggctagtggaatttttggcacttggtcctgctgcaagttacataactggacaggtttcactttcacctactatga

SEQ ID NO: 35 - protein, 322 aa, Cannabis sativa
MAAIAGSNVVVSKSTGISSSFFDRKAPHFRQWSPISGGQSLRHSISLQCKSKSSSGGVVKAQGASLEETSAGVGQNVEAPVVIVTGASRGIGKATALALGKAGCKVLVNYARSSKEAEEVSKEIEASGGQAITFGGDVSKEADVELMIKTAVDAWGTIDVLVNNAGITRDNLIMRMKKSQWQDVIDLNLTGVFLCTQAAAKIMMKKKKGRIINVASVFGLVGNAGQANYSAAKAGVIGFTKAIAKEYSSRNITVNAIAPGFIASDMTAKLGDDIEKKNLEGIPLGRYGQPEEVAGLVEFLALGPAASYITGQVLTIDGGMVM

SEQ ID NO: 36 - protein, 322 aa, Cannabis sativa
MAAIAGSNVVVSKSTRISSSFFDRKAPHFRQWSPIFGGQSLRHSISLQCKSKSSSGGVVKAQGASLEETSAGVGQNVEAPVVIVTGASRGIGKATALALGKAGCKVLVNYARSSKEAEEVSKEIEASGGQAITFGGDVSKEADVELMIKTAVDAWGTIDVLVNNAGITRDNLIMRMKKSQWQDVIDLNLTGVFLCTQAAAKIMMKKKKGRIINVASVFGLVGNAGQANYSAAKAGVIGFTKAIAKEYSSRNITVNAIAPGFIASDMTAKLGDDIEKKNLEGIPLGRYGQPEEVAGLVEFLALGPAASYITGQVLTIDGGMVM

SEQ ID NO: 37 - protein, 322 aa, Cannabis sativa
MAAIAGSNVVVSKSTRISSSFFDRKAPHFRQWSPISSGQSLRHSISLQCKSKSSSGGVVKAQGASLEETSAGVGQNVEAPVVIVTGASRGIGKATALALGKAGCKVLVNYARSSKEAEEVSKEIEASGGQAITFGGDVSKEADVELMIKTAVDAWGTIDVLVNNAGITRDNLIMRMKKSQWQDVIDLNLTGVFLCTQAAAKIMMKKKKGRIINVASVFGLVGNAGQANYSAAKAGVIGFTKAIAKEYSSRNITVNAIAPGFIASDMTAKLGDDIEKKNLEGIPLGRYGQPEEVAGLVEFLALGPAASYITGQVLTIDGGMVM

SEQ ID NO: 38 - protein, 322 aa, Cannabis sativa
MAAIAGSNVVVSKSTRISSSFFDRKAPHFRQWSPISGGQSLRHSISLQCKSKSSSGGVVKAQVASLEETSAGVGQNVEAPVVIVTGASRGIGKATALALGKAGCKVLVNYARSSKEAEEVSKEIEASGGQAITFGGDVSKEADVELMIKTAVDAWGTIDVLVNNAGITRDNLIMRMKKSQWQDVIDLNLTGVFLCTQAAAKIMMKKKKGRIINVASVFGLVGNAGQANYSAAKAGVIGFTKAIAKEYSSRNITVNAIAPGFIASDMTAKLGDDIEKKNLEGIPLGRYGQPEEVAGLVEFLALGPAASYITGQVLTIDGGMVM

SEQ ID NO: 39 - protein, 322 aa, Cannabis sativa
MAAIAGSNVVVSKSTRISSSFFDRKAPHFRQWSPISGGQSLRHSISLQCKSKSSSGGVVKAQGGSLEETSAGVGQNVEAPVVIVTGASRGIGKATALALGKAGCKVLVNYARSSKEAEEVSKEIEASGGQAITFGGDVSKEADVELMIKTAVDAWGTIDVLVNNAGITRDNLIMRMKKSQWQDVIDLNLTGVFLCTQAAAKIMMKKKKGRIINVASVFGLVGNAGQANYSAAKAGVIGFTKAIAKEYSSRNITVNAIAPGFIASDMTAKLGDDIEKKNLEGIPLGRYGQPEEVAGLVEFLALGPAASYITGQVLTIDGGMVM

SEQ ID NO: 40 - protein, 322 aa, Cannabis sativa
MAAIAGSNVVVSKSTRISSSFFDRKAPHFRQWSPISGGQSLRHSISLQCKSKSSSGGVVKAQGATLEETSAGVGQNVEAPVVIVTGASRGIGKATALALGKAGCKVLVNYARSSKEAEEVSKEIEASGGQAITFGGDVSKEADVELMIKTAVDAWGTIDVLVNNAGITRDNLIMRMKKSQWQDVIDLNLTGVFLCTQAAAKIMMKKKKGRIINVASVFGLVGNAGQANYSAAKAGVIGFTKAIAKEYSSRNITVNAIAPGFIASDMTAKLGDDIEKKNLEGIPLGRYGQPEEVAGLVEFLALGPAASYITGQVLTIDGGMVM

SEQ ID NO: 41 - protein, 322 aa, Cannabis sativa
MAAIAGSNVVVSKSTRISSSFFDRKAPHFRQWSPISGGQSLRHSISLQCKSKSSSGGVVKAQGASLEETSAGVGQNLEAPVVIVTGASRGIGKATALALGKAGCKVLVNYARSSKEAEEVSKEIEASGGQAITFGGDVSKEADVELMIKTAVDAWGTIDVLVNNAGITRDNLIMRMKKSQWQDVIDLNLTGVFLCTQAAAKIMMKKKKGRIINVASVFGLVGNAGQANYSAAKAGVIGFTKAIAKEYSSRNITVNAIAPGFIASDMTAKLGDDIEKKNLEGIPLGRYGQPEEVAGLVEFLALGPAASYITGQVLTIDGGMVM

SEQ ID NO: 42 - protein, 322 aa, Cannabis sativa
MAAIAGSNVVVSKSTRISSSFFDRKAPHFRQWSPISGGQSLRHSISLQCKSKSSSGGVVKAQGASLEETSAGVGQNVEAPVVIVTGASRGIRKATALALGKAGCKVLVNYARSSKEAEEVSKEIEASGGQAITFGGDVSKEADVELMIKTAVDAWGTIDVLVNNAGITRDNLIMRMKKSQWQDVIDLNLTGVFLCTQAAAKIMMKKKKGRIINVASVFGLVGNAGQANYSAAKAGVIGFTKAIAKEYSSRNITVNAIAPGFIASDMTAKLGDDIEKKNLEGIPLGRYGQPEEVAGLVEFLALGPAASYITGQVLTIDGGMVM

SEQ ID NO: 43 - protein, 322 aa, Cannabis sativa
MAAIAGSNVVVSKSTRISSSFFDRKAPHFRQWSPISGGQSLRHSISLQCKSKSSSGGVVKAQGASLEETSAGVGQNVEAPVVIVTGASRGIGKATALALGKAGCKVLVNYAKSSKEAEEVSKEIEASGGQAITFGGDVSKEADVELMIKTAVDAWGTIDVLVNNAGITRDNLIMRMKKSQWQDVIDLNLTGVFLCTQAAAKIMMKKKKGRIINVASVFGLVGNAGQANYSAAKAGVIGFTKAIAKEYSSRNITVNAIAPGFIASDMTAKLGDDIEKKNLEGIPLGRYGQPEEVAGLVEFLALGPAASYITGQVLTIDGGMVM

SEQ ID NO: 44 - protein, 322 aa, Cannabis sativa
MAAIAGSNVVVSKSTRISSSFFDRKAPHFRQWSPISGGQSLRHSISLQCKSKSSSGGVVKAQGASLEETSAGVGQNVEAPVVIVTGASRGIGKATALALGKAGCKVLVNYARSSKEAEEVSKEIEASGGQAITFGGDISKEADVELMIKTAVDAWGTIDVLVNNAGITRDNLIMRMKKSQWQDVIDLNLTGVFLCTQAAAKIMMKKKKGRIINVASVFGLVGNAGQANYSAAKAGVIGFTKAIAKEYSSRNITVNAIAPGFIASDMTAKLGDDIEKKNLEGIPLGRYGQPEEVAGLVEFLALGPAASYITGQVLTIDGGMVM

SEQ ID NO: 45 - protein, 322 aa, Cannabis sativa
MAAIAGSNVVVSKSTRISSSFFDRKAPHFRQWSPISGGQSLRHSISLQCKSKSSSGGVVKAQGASLEETSAGVGQNVEAPVVIVTGASRGIGKATALALGKAGCKVLVNYARSSKEAEEVSKEIEASGGQAITFGGDVSKEADVELMTKTAVDAWGTIDVLVNNAGITRDNLIMRMKKSQWQDVIDLNLTGVFLCTQAAAKIMMKKKKGRIINVASVFGLVGNAGQANYSAAKAGVIGFTKAIAKEYSSRNITVNAIAPGFIASDMTAKLGDDIEKKNLEGIPLGRYGQPEEVAGLVEFLALGPAASYITGQVLTIDGGMVM

SEQ ID NO: 46 - protein, 322 aa, Cannabis sativa
MAAIAGSNVVVSKSTRISSSFFDRKAPHFRQWSPISGGQSLRHSISLQCKSKSSSGGVVKAQGASLEETSAGVGQNVEAPVVIVTGASRGIGKATALALGKAGCKVLVNYARSSKEAEEVSKEIEASGGQAITFGGDVSKEADVELMIKIAVDAWGTIDVLVNNAGITRDNLIMRMKKSQWQDVIDLNLTGVFLCTQAAAKIMMKKKKGRIINVASVFGLVGNAGQANYSAAKAGVIGFTKAIAKEYSSRNITVNAIAPGFIASDMTAKLGDDIEKKNLEGIPLGRYGQPEEVAGLVEFLALGPAASYITGQVLTIDGGMVM

SEQ ID NO: 47 - protein, 322 aa, Cannabis sativa
MAAIAGSNVVVSKSTRISSSFFDRKAPHFRQWSPISGGQSLRHSISLQCKSKSSSGGVVKAQGASLEETSAGVGQNVEAPVVIVTGASRGIGKATALALGKAGCKVLVNYARSSKEAEEVSKEIEASGGQAITFGGDVSKEADVELMIKTAVDAWGTIDVLVNNAGITRDNLMMRMKKSQWQDVIDLNLTGVFLCTQAAAKIMMKKKKGRIINVASVFGLVGNAGQANYSAAKAGVIGFTKAIAKEYSSRNITVNAIAPGFIASDMTAKLGDDIEKKNLEGIPLGRYGQPEEVAGLVEFLALGPAASYITGQVLTIDGGMVM

SEQ ID NO: 48 - protein, 322 aa, Cannabis sativa
MAAIAGSNVVVSKSTRISSSFFDRKAPHFRQWSPISGGQSLRHSISLQCKSKSSSGGVVKAQGASLEETSAGVGQNVEAPVVIVTGASRGIGKATALALGKAGCKVLVNYARSSKEAEEVSKEIEASGGQAITFGGDVSKEADVELMIKTAVDAWGTIDVLVNNAGITRDNLIMRMKKSQWQDVIDLNLTGVFLCTQAAAKIMMKKKKGRIINVASVFGLVGNAGQANCSAAKAGVIGFTKAIAKEYSSRNITVNAIAPGFIASDMTAKLGDDIEKKNLEGIPLGRYGQPEEVAGLVEFLALGPAASYITGQVLTIDGGMVM

SEQ ID NO: 49 - protein, 322 aa, Cannabis sativa
MAAIAGSNVVVSKSTRISSSFFDRKAPHFRQWSPISGGQSLRHSISLQCKSKSSSGGVVKAQGASLEETSAGVGQNVEAPVVIVTGASRGIGKATALALGKAGCKVLVNYARSSKEAEEVSKEIEASGGQAITFGGDVSKEADVELMIKTAVDAWGTIDVLVNNAGITRDNLIMRMKKSQWQDVIDLNLTGVFLCTQAAAKIMMKKKKGRIINVASVFGLVGNAGQANYSAAKAGVIGFTKAVAKEYSSRNITVNAIAPGFIASDMTAKLGDDIEKKNLEGIPLGRYGQPEEVAGLVEFLALGPAASYITGQVLTIDGGMVM

SEQ ID NO: 50 - protein, 322 aa, Cannabis sativa
MAAIAGSNVVVSKSTRISSSFFDRKAPHFRQWSPISGGQSLRHSISLQCKSKSSSGGVVKAQGASLEETSAGVGQNVEAPVVIVTGASRGIGKATALALGKAGCKVLVNYARSSKEAEEVSKEIEASGGQAITFGGDVSKEADVELMIKTAVDAWGTIDVLVNNAGITRDNLIMRMKKSQWQDVIDLNLTGVFLCTQAAAKIMMKKKKGRIINVASVFGLVGNAGQANYSAAKAGVIGFTKAIAKEYSSRNITVNAIAPGFIASDMTAKLGDDIEKKNLEGIPLGRYGQPEEVAGLVEFLALDPAASYITGQVLTIDGGMVM